(19) United States
(12) Patent Application Publication
(10) Pub. No.: US 2016/0195536 A1
(43) Pub. Date: Jul. 7, 2016

(54) Materials and Methods Relating to Pancreatic Cancer

(71) Applicants: Electrophoretics Limited, Cobham, Surrey (GB); King's College Hospital NHS Foundation Trust, London (GB)

(72) Inventors: Yoh Zen, Kobe (JP); Nigel Heaton, London (GB); Alberto Quaglia, London (GB); David Britton, Cobham, Surrey (GB); Malcolm Ward, Cobham,Surrey (GB); Ian Pike, Cobham, Surrey (GB); Vikram Mitra, Cobham, Surrey (GB)

(73) Assignees: Electrophoretics Limited, Surrey (GB); King's College Hospital NHS Foundation Trust, London (GB)

(21) Appl. No.: 14/912,299

(22) PCT Filed: Aug. 13, 2014
(86) PCT No.: PCT/GB2014/052475

§ 371 Date: Feb. 16, 2016

(30) Foreign Application Priority Data
Aug. 13, 2013 (GB) 1314485.2

(51) Int. Cl. G01N 33/574; C07K 2/00; A61K 31/506

(52) U.S. Cl. CPC G01N 33/57438; A61K 31/506; C07K 2/00; G01N 2440/14; G01N 2570/00; G01N 2560/00; G01N 2458/15

(57) ABSTRACT

The present invention concerns materials and methods relating to pancreatic cancer and personalized medicine as applied to pancreatic cancer. Particularly, the invention relates to materials and methods for the determination of significantly modulated protein phosphorylation and/or expression as well as the activity of signalling pathways collectively providing a tumour profile that can guide selection of the most appropriate treatment regime based on the likelihood of tumour recurrence or the identity of activated drug targets in pancreatic cancer.

### FIELD OF THE INVENTION

The present invention concerns materials and methods relating to pancreatic cancer and personalised medicine as applied to pancreatic cancer. Particularly, the invention relates to materials and methods for the determination of significantly modulated protein phosphorylation and/or expression as well as the activity of signaling pathways collectively providing a tumour profile that can guide selection of the most appropriate treatment regime based on the likelihood of tumour recurrence or the identity of activated drug targets in pancreatic cancer tissue.

### BACKGROUND OF THE INVENTION

Protein phosphorylation is a common process modulating the activity of oncogenic and tumor suppressor proteins [1-3]. In many cases, phosphorylation results in switch-like changes in protein function due to modulation of protein folding, substrate affinity, stability, and activity of its substrates, in turn affecting signaling pathways controlling cell proliferation, migration, differentiation, and apoptosis. Dysregulation of phosphorylation can thus contribute to the cancer phenotype [4] and provides a potential source of new drug targets, diagnostic and prognostic biomarkers that significantly cannot be measured using genomic methods. Pancreatic cancer is one of the most aggressive malignant neoplasms with a median survival of 6 months post-diagnosis. In part this is a result of the fact that a significant proportion of patients are diagnosed at an advanced stage where treatment options are very limited [5]. As is the case for other cancers, molecular targeting therapy is promising for treatment of advanced or recurrent pancreatic cancer [6]. Although a variety of molecular targeting drugs have been available in the last decade and many others are also expected in the next few years, a breakthrough is still required for prediction of drug effects and drug selection. For example, sorafenib, a multi-kinase inhibitor acting on hyperactive vascular endothelial growth factor receptor, platelet-derived growth factor receptor and Raf, has proven efficacy in some patients with advanced hepatocellular carcinoma [7], but response rates remain frustratingly low as there are currently no pathway activity tests that can predict its effect in an individual patient before starting treatment.

It has long been recognised that chemotherapy, even with highly selective molecular targeting medicines will ultimately fail due to acquired resistance. Typically this is driven by the switching from one oncogenic pathway to another under the selective pressure of the drug treatment. As an example, the V600E mutation of B-Raf is a common feature in aggressive melanoma leading to hyperactivation of the Raf signalling pathway. Highly selective inhibitors of V600E B-Raf were rapidly developed and approved based on dramatic initial treatment response. However, the vast majority of patients ultimately relapse, despite B-Raf signalling being silenced, through a range of different mechanisms involving aberrant dimerization, Raf isoform switching and alternative activation of MEK and ERK. A proposed solution for such patterns of acquired resistance is the administration of multiple molecular targeting drug combinations which each may not be sufficient to kill the tumour, but which collectively act to block evolving resistance. This strategy has been termed ‘synthetic lethality’.

### SUMMARY OF THE INVENTION

The inventors have recognised a need for a reliable and time and cost-effective means for defining the optimal drug combination for treating pancreatic cancer and for the prediction of and monitoring for drug resistance in such tumours.

Accordingly, the inventors set out to establish an analytical approach to help drug selection, where expression and activity of multiple drug targets are comprehensively assessed on a case-by-case basis. Phosphorylation is a key event modulating protein activity, therefore measuring protein phosphorylation is a useful indicator of activation status.

There are hundreds of anti-cancer drug targets and thousands of oncogenic signaling proteins and measuring expression and activation status of all of these using immunohistochemistry (IHC), the current gold standard analysis, to guide optimal treatment selection is not feasible. Reverse phase protein microarrays (RPMA) have the potential to offer broader coverage than IHC but have limitations due to a currently small repertoire of phosphorylation site-specific antibodies and poor specificity/cross reactivity. Since the prime regulatory processes controlling oncoprotein activity are post-translational modifications, genomics-based technologies cannot provide an alternative solution. Previously liquid chromatography-mass spectrometry (LC-MS/MS) based proteomic approaches have been developed to identify and quantify thousands of proteins and their phosphorylation sites [8, 9] and the inventors have now successfully adapted and applied these methods to the analysis of oncogenic signalling pathways to identify the optimal drug targets expressed within an individual tumour.

The inventors have developed a new LC-MS/MS based proteomic workflow to overcome many of the technical and bio-informatic difficulties involved in effectively identifying and quantifying activated proteins, activated signaling pathways, and activated drug targets, at a global or system wide level on a case by case basis. In specific terms, the inventors provide a high-density phospho-proteomic workflow applicable to experimental cancer cell lines, xenograft tumour tissue and clinical tissue using isotopic and/or isobaric mass tag labelling enabling the analysis of multiple samples simultaneously [10, 11]. Preferably two or more samples are analysed simultaneously. Most preferably at least 10 samples can be analysed together. Samples may be paired tissues from the tumour and adjacent healthy tissue from individual patients or from more than one patient, e.g. at least two, at least three, or at least 4. Most preferably paired tumour and healthy tissues from 5 patients are analysed together in a single 10-plex experiment.

It is a particular feature of the present invention that, given the large amount of data generated for each individual patient, a system for data storage, retrieval and analysis is provided. In particular the inventors provide a database and suite of data analysis tools to extract relevant biological information from their complex dataset.

Specifically, the inventors have applied their global phospho-proteomic workflow (SysQuant) to compare cancerous and non-cancerous pancreatic tissue. This phosphoproteomic workflow allows simultaneous measurements of multiple phosphoproteins and provides rapid measure of signaling pathway activity in a sample. This workflow has enabled the inventors to identify signaling pathways and drug targets that show significant modulation in expression and activity between cancerous and non-cancerous tissue types at an average level across all pancreatic cancer cases to determine common drivers of the pancreatic cancer phenotype. The inventors were also able to interrogate the entire database to identify different combinations of molecular events contributing to the cancer phenotype which were unique to an individual case or subgroups. Accordingly, this workflow provides for the first time a way of not only diagnosing pancreatic cancer, but more importantly stratifying patients into different treatment regimens based on the activation status of these newly determined targets on a case by case basis.

In addition, measuring the phosphopeptide molecular profile allows for the first time a prognostic tool for pancreatic cancer. Hierarchal clustering of phosphopeptide abundance separated patients into groups based on recurrence and non-recurrence. This led to the identification of many prognostic phosphopeptide and thus their respective phosphoprotein markers which form independent aspects of the present invention.

The approach taken by the inventors allowed simultaneous measurement of more than 5000 phosphorylation sites of more than 2000 proteins in tumor versus background pancreatic tissue from patients with pancreatic head adenocarcinoma. Many of these were determined to be modulatory phosphorylation sites known to affect activity of drug targets such as FYN, GSK3α/β, HDAC1/2, the RAF kinases, MAPKs (p38 and ERK2), AKT, PKCs, Casein Kinases and others.

The inventors determined the relative abundance of proteins in tumor (T) compared to non-tumor (NT) tissue, using median log₂ T/NT ratios of the non-phosphorylated peptides unique to each protein as surrogates to calculate the relative abundance of the respective proteins.

From this information, they found it was possible to develop a predictive algorithm to assign tissue samples to tumour or non-tumour phenotype, i.e. as a diagnostic aid. Further, they found that the differentially activated pathway proteins can be used as therapeutic targets. That is, drugs may be developed which are capable, either directly or indirectly, of regulating the expression, activation or inhibition of the proteins of interest as appropriate towards those levels found in normal healthy tissue.

Having created a comprehensive database of individual phosphorylation site status across thousands of proteins, the invention provides for the first time the means for a number of additional analyses to be performed. For example, the ability to predict the likelihood and potential timing of tumour recurrence provides a major benefit in designing the optimal treatment strategy. Using hierarchical clustering analysis of the data, the inventors were surprisingly able to categorise tumours into recurrent and non-recurrent phenotypes independently of any other clinical data. Even more surprisingly, a subset of protein phosphorylation sites were highly correlated with recurrence and each of these represents a novel therapeutic target or marker in pancreatic cancer. Thus, the inventors also provide new therapeutic targets to enable the development of molecular targeting drugs for the treatment of pancreatic cancer.

In a yet further aspect of the present invention, one or more of the regulated protein phosphorylation sites associated with the recurrent pancreatic cancer phenotype represent novel biomarkers for the diagnosis and prognosis of recurrent pancreatic cancer. In accordance with this aspect of the invention means of detecting and/or quantifying phosphorylation at the one or more sites are provided. Such methods include but are not limited to immunohistochemistry, Western blotting, ELISA and mass spectrometry.

To ascertain relative activation status of kinases, other enzymes and other classes of proteins in tumor compared to non-tumor tissue in each case, the inventors used relative abundance of phosphopeptides containing phosphorylation sites known to either induce enzyme activation or inhibition. Table 15 provides all phosphopeptides displaying log₂ T/NT ratios ≧1 or ≦−1 that contain phosphorylation sites that are known to either induce activation or inhibition of the phosphorylated enzyme, in each case.

In addition to determining which proteins and phosphopeptides demonstrated significant differences in abundance between tumor and non-tumor tissue when averaged across all cases, the inventors have also determined which phosphopeptides were highly modulated within each individual patient and provide herein markers and targets for the diagnosis and prognosis, including prediction of recurrence and drug resistance, of pancreatic cancer.

For example, the inventors have determined the relative activation status of; Glycogen synthase kinase-3 alpha and beta, Histone deacetylase 1 and 2, RAF proto-oncogene serine/threonine-protein kinase, Serine/threonine-protein kinase A-Raf, Dual specificity mitogen-activated protein kinase kinase 6, Mitogen-activated protein kinase 14 (p38 MAPK), and over 20 others (see e.g. Table 4 and Table 15).

The inventors further provide examples which demonstrate how their LC-MS workflow, can simultaneously measure the abundance and activity of 1000's of signaling and structural proteins in tumor tissue relative to non-tumor tissue, and show how such measurements can be used to better understand the molecular events leading to cancer and therefore guide selection of the most suitable inhibitory agents to treat a patient on an individual basis using one, or a combination of approved or experimental molecular targeting medicines. Critically, the inventors have demonstrated using hierarchal clustering of phosphopeptide log₂ T/NT ratios that they can identify those patients more likely to show recurrence of pancreatic cancer compared to those patients less likely to show recurrence at the same time point.

Accordingly, at its most general, the invention provides materials and methods for the diagnosis, prognosis and treatment (including the selection of targeted therapies) of pancreatic cancer arising from the identification of signaling pathways and drug targets that show significant modulation in expression and activity between cancerous and non-cancerous tissue types. The data provided herein shows the molecular events driving the cancer phenotype on a case by case basis and for the first time provides the means for clinicians to predict not only the most effective targeted therapy, but also predict likelihood of recurrence of pancreatic cancer.

In a first aspect, there is provided a pancreatic tumor classification system comprising a pancreatic tumour classification apparatus and an information communication terminal apparatus, said pancreatic tumor classification apparatus including a control component and a memory component, said apparatuses being communicatively connected to each other via a network;
(1) wherein the information communication terminal apparatus includes
(1a) a protein data sending unit that transmits the protein data derived from a pancreatic tumor sample of a subject to the pancreatic tumor classification apparatus;
(1b) a result-receiving unit that receives the result of the pancreatic tumor classification of the subject transmitted from the pancreatic tumour classification apparatus;
(2) wherein the pancreatic tumor classification apparatus includes
(2a) a protein data-receiving unit that receives protein data derived from the pancreatic tumor sample of the subject transmitted from the information communication terminal apparatus;
(2b) a data comparison unit which compares the data from the data-receiving unit with the data stored in the memory unit;
(2c) a classifier unit that determines the class (e.g. molecular phenotype) of the pancreatic tumour of the subject, based on the results of the data comparison unit; and
(2d) a classification result-sending unit that transmits the classification result of the subject obtained by the classifier unit to the information communication terminal apparatus; and
wherein the memory unit contains protein expression level and/or phosphorylation data of at least one (preferably a plurality) proteins selected from Tables 2, 3, 4, 11, 12, 13 and/or 15.

The memory unit may contain protein expression level and/or phosphorylation data of at least one or a plurality of proteins selected from each of Tables 2, 3, 4, 11, 12, 13 and/or 15. That is, the memory unit may contain data from two more proteins from Table 2 in combination with data from two more, three or more, four or more, five or more proteins from Table 3, 4, 11, 12, 13 and/or 15; or any combination thereof. This combination of proteins from Tables 2, 3, 4, 11, 12, 13 and/or 15 is applicable to each and every aspect of the invention described herein.

The data derived from the pancreatic tumor sample of the subject is preferably expression level data and/or phosphorylation status data, such as that obtained from methods described herein e.g. LC-MS/MS and other proteomic approaches. The data may be derived just from the tumor (or suspected tumor) sample, but in preferred embodiments, a second data set derived from non-tumor (background) pancreatic tissue of the same subject may also be provided.

The protein data received by the data-receiving unit may be the actual protein or phosphoprotein levels, or it may be peptide or phosphopeptide levels from which the protein or phosphoprotein levels can be calculated. The peptide or phosphopeptide is unique to the at least one (preferably plurality) protein or phosphoprotein. In some embodiments it is preferable to use multiple, i.e. 2, 3, 4, or 5 peptides which are all unique to said protein. Where multiple peptides are used, data may be collated and optionally a median value used in the data comparison step.

The memory unit preferably includes data sets relating to protein expression levels and/or phosphoprotein levels representative of pancreatic tumor. In a preferred embodiment, the protein expression levels and/or phosphoprotein levels are derived from actual peptide or phosphopeptide levels in the sample. This is particularly so if the data has been obtained using proteomic methods such as the LC-MS/MS method described herein. The data sets may provide a representative (e.g. average) level of protein expression levels or phosphoprotein levels found in pancreatic tumors from a collection of data sets, e.g. as provided herein by Table 12. Alternatively, it may be preferable for the data sets to include a value representing a ratio of the protein expression level or phosphoprotein level as compared to the protein expression level or phosphoprotein level of background (i.e. non-tumor) tissue obtained from the same source. By way of example, this value is presented herein as Log2 T/NT.

In addition to confirming that the sample is a pancreatic tumor, the data sets held in the protein data-storing unit allow the system to classify the tumor into recurrence or non-recurrence classes. By inputting the data representative of phosphoprotein levels of the pancreatic tissue sample taken from a subject, and optionally, data representative of phosphoprotein levels of background pancreatic tissue taken from the same subject, the data comparison unit may compare this data with a data set including at least data relating to a plurality of proteins selected from Table 11 held in the memory unit.

In one embodiment, there is provided a method of predicting the likelihood of recurrence of a pancreatic tumor in a subject after treatment, said method comprising detecting the level of phosphorylation of at least one protein selected from the group consisting of Homeodomain-interacting protein kinase I (HIPK1); Serine/threonine-protein kinase MRCK alpha (MRCK alpha); and myosin light chain kinase, smooth muscle (MLCK) in a tumour sample obtained from said subject, wherein elevated levels of phosphorylation compared to background (non-tumor) levels is indicative of the likelihood of tumor recurrence.

In this way, the system can compare the phosphoprotein levels obtained from pancreatic tumor sample with phosphoprotein levels representative of a tumor recurrence phenotype for the same protein and thereby classify the tumor as either a tumor with likelihood of recurrence or likelihood of non-recurrence.

In a preferred embodiment the comparison of phosphoprotein levels may also provide a prediction of timing of tumor recurrence, e.g. between 8 and 33 months, between 10 and 20 months or between 15 and 17 months after removal of the tumors.

The pancreatic tumor classification system described above may also be used to classify a pancreatic tumor based on drug susceptibility. In this embodiment, the memory unit may contain, at least phosphoprotein data of a plurality of proteins selected from Table 15 or Table 4.

For example, the inventors have determined those phosphoproteins which are up-regulated or down-regulated in pancreatic tumor (and/or have differences in phosphorylation status) compared to normal pancreatic tissue, and from these have identified those that contain phosphorylation sites that are known to either induce activation or inhibition of the phosphorylated protein (e.g. enzyme). (See Table 15 and Table 4).

Accordingly, by comparing the phosphoprotein levels of a pancreatic tumor sample with the phosphoprotein levels of a plurality of proteins selected from Table 15 and/or Table 4, it is possible for the system to classify the tumor on the basis of drug susceptibility. The drugs may be selected from GSK2141795, GSK2141796, GSK214179, Dasatinib, AEZS-131, Vorinostat, and Sorafenib.

In some cases, the phosphoprotein levels of the sample are compared with those for one or more, two or more, three or more, or all of the following proteins: Glycogen Synthase kinase-3 alpha and beta, Histone deacetylase I and 2, RAF proto-oncogene serine/threonine-protein kinase, serine/threonine-protein kinase A-Raf, Dual specificity mitogen-activated protein kinase kinase 6, mitogen-activated protein kinase 14 (p38 MAPK).

The pancreatic tumor classification system may be used to determine tumor or non-tumor phenotype of the sample obtained from the subject where the memory unit contains data relating to protein expression levels of a plurality of proteins selected from Table 12 or Table 2.

As a result, the system can compare the expression levels of proteins determined from the sample with expression levels held in the memory unit that are representative of pancreatic tumor. In this way, the sample can be identified as tumor or non-tumor.

Although the inventors acknowledge that the system may be used to perform independent classification of phenotypes, i.e. tumor v non-tumor, recurrence phenotype v non-recurrence phenotype, drug susceptibility profile, and primary tumour v secondary (metastatic tumor), it is preferred that the data contained within the memory unit of the system will allow a sample to be classified as multiple phenotypes, e.g. tumor, predicted recurrence and drug susceptibility profile.

In a preferred embodiment, the system further comprises the means to add the inputted data via the data sending unit to the stored data already held in the memory unit so that this new data can be included in the analysis performed by the determining unit. In this way the data representative of pancreatic tumor molecular phenotypes is constantly updated.

In a preferred embodiment, the pancreatic tumor classification system is connected to an apparatus for determining protein expression levels or protein phosphorylation levels in a pancreatic tumor sample and feeding this data to the protein data sending unit.

Ideally the apparatus can process multiple samples using LC-MS/MS as described herein.

In accordance with this first aspect of the invention, there is also provided a pancreatic tumor cellular classification program that makes an information processing apparatus including a control component and a memory component execute a method of determining and/or classifying the pancreatic tumor of a subject, the method comprising:
(i) a comparing step of comparing data based on the protein expression levels and/or protein phosphorylation levels of at least one (preferably a plurality) protein selected from Tables 2, 3, 4, 11, 12, 13 and/or 15 obtained of a subject with the protein expression level data and/or the protein phosphorylation data stored in the memory component; and
(ii) a classifying step for classifying the pancreatic tumor cells of said subject, based on the comparison calculated at the comparing step; and wherein said tumor is classified into phenotypes including tumor, non-tumor; tumor recurrence, tumor non-recurrence; primary tumour, secondary (metastatic tumor) and/or drug susceptibility.

In accordance with this aspect of the invention, there is also provided a computer-readable recording medium, comprising the pancreatic tumour classification program described above recorded thereon.

The data representing protein expression levels and/or protein phosphorylation levels (i.e. amount of phosphorylated protein) may be derived from peptide levels and/or phosphopeptide levels in the sample where said peptides and/or phosphopeptides are each unique to a particular protein selected from the specified Tables. Example peptides and phosphopeptides are provided in the Tables for each protein. However, it will be appreciated that other peptides and phosphopeptides may be designed which will also be unique for the protein from which they are derived, e.g. by proteolytic enzyme digestion such as trypsin, aspN, gluC and other such enzymes well known in the art.

In respect of all aspects of the invention described herein, the sample from which the protein data is derived may be obtained from a subject already diagnosed with pancreatic cancer or it may be obtained from a subject suspected of having pancreatic cancer. Accordingly, with regard to the latter, the classification of the cancer may also include the diagnosis.

In a second aspect of the invention, there is provided a method of diagnosing pancreatic cancer in a subject comprising determining the modulation of one or more, or a plurality of proteins and/or phosphorylation sites selected from Table 12 and/or Table 2, Table 15 and/or Table 3 in a biological sample obtained from said subject, wherein
(a) the presence of said one or more, or plurality of proteins in said sample is indicative of the subject having pancreatic cancer;
(b) the amount (concentration) of said one or more, or plurality of proteins as compared to a reference amount for said one or more, or plurality of proteins is indicative of the subject having pancreatic cancer;
(c) a change in amount (concentration) of said one or more, or plurality of proteins as compared to a reference amount for said one or more, or plurality of proteins is indicative of the subject having pancreatic cancer; or
(d) a change in phosphorylation status of said one or more, or plurality of proteins as compared to a reference status for said one or more, or plurality of proteins is indicative of the subject having pancreatic cancer.

In a third aspect, the invention provides a method of classifying a pancreatic tumour into molecular phenotypes selected from the group consisting of tumor, non-tumor, recurrence, non-recurrence, drug susceptibility, primary tumor and secondary (metastatic) tumor, said method comprising
(1) determining expression levels and/or protein phosphorylation level of a plurality of proteins in a biological sample obtained from said subject;
(2) producing an expression level and/or phosphoprotein profile for said sample;
(3) comparing said subject profile with a reference profile representative of the pancreatic tumour molecular phenotype(s); and
(4) determining the molecular phenotype of pancreatic tumour based on the comparison between the subject profile and the reference profile;
wherein the plurality of proteins are selected from a biomarker panel as represented by Table 2, 3, 4, 11, 12, 13 and/or 15.

For this and all other aspects of the invention, the reference protein expression levels and/or protein phosphorylation level profile may be determined from non-tumor pancreatic tissue from the same subject. In this way, the difference in protein expression levels and/or protein phosphorylation levels may be used to determine the molecular phenotype of the pancreatic tumor. Alternatively, the reference levels may be a database comprising data representing expression levels and/or phosphorylation levels for the proteins of interest as selected from any one or more of Tables 2, 3, 4, 11, 12, 13 and 15. Ideally, the reference levels are provided by a pancreatic tumor classification system according to the first aspect. The data representing expression levels and/or protein levels may be a collection of data obtained from multiple tumor samples and presented as an average or range. The data may relate to the levels of specific peptides and/or phosphopeptides each being unique to a protein of interest.

In a fourth aspect of the invention, there is provided a method of selecting a treatment regime for a subject suffering from pancreatic cancer, said method comprising
(1) determining expression levels and/or phosphorylation of one or more, or a plurality of proteins in a biological sample obtained from said subject;
(2) comparing said expression levels and/or phosphorylation status with reference expression levels and/or phosphorylation levels for said one or more, or plurality of proteins, said reference levels representative of pancreatic tumour molecular phenotypes selected from tumor, non-tumor; tumor recurrence, tumor non-recurrence; primary tumor, secondary (metastatic) tumor and/or drug susceptibility;
(3) determining the molecular of pancreatic tumour based on the comparison between the expression levels and/or phosphorylation levels of the proteins in the biological sample and the reference expression levels; and
(4) selecting a treatment regime on the basis of the molecular phenotype of pancreatic tumour,
wherein the plurality of proteins are selected from a biomarker panel as represented by Table 2, 3, 4, 11, 12, 13 and/or 15.

The biological sample is preferably a sample of the pancreatic tumor (e.g. a biopsy), but it is envisaged that for this and other aspects of the invention, the biological sample could be any fluid or solid sample of the subject that was capable of providing a representation of the proteins regulated in pancreatic tumor. For example, biological markers as identified herein may be determined and their amount or concentration, or phosphorylation status, quantified from a blood or urine sample from the subject, thereby avoiding the need for a biopsy.

The method may, for example, allow the user to determine whether the pancreatic sample obtained from the subject is tumor, has a likelihood of recurrence, (i.e. between 8 and 33 months, between 10 and 20 months or between 15 and 17 months after removal of the tumor) and/or what drug targets are present in the tumor.

For example, by comparison with the reference expression levels, the method may identify a plurality of up-regulated proteins selected from Table 12, or more preferably selected from Table 2. In still preferred embodiments, these up-regulated proteins include at least Homeodomain-interacting protein kinase-1 and/or Mucin 1; optionally in combination with any one, two, three, four or more further proteins selected from Table 12 and/or 2. The presence of these up-regulated proteins as compared to the reference level will indicate that the sample is pancreatic tumor.

Likewise, the method may determine those proteins with phosphorylation sites which are significantly regulated compared to references levels, i.e. by comparing the levels of a plurality of phosphorylated proteins with reference levels selected from Table 3, 11, 4, 13 and/or 15. This comparison allows the sample to be classified into the phenotype tumor with a likelihood of recurrence or the phenotype tumor with a non-likelihood of recurrence. For example, the plurality of proteins with regulated phosphorylation sites may be selected from Table 11 or, more preferably, from Table 11A (up-regulated phosphorylation in recurrent tumors) and Table 11B (down-regulated phosphorylation in recurrent tumors).

In fact, the results obtained by the present inventors suggest that the up-regulation in phosphorylation of Dual specificity mitogen-activated protein kinase kinase 2 alone may be sufficient to predict the likelihood of recurrence in a tumor between 8 and 33 months, between 10 and 20 months or between 15 and 17 months after removal of the tumor. Accordingly, the determination of increased phosphorylation of Dual specificity mitogen-activated protein kinase kinase 2 in a biological sample obtained from a subject in order to predict likelihood of recurrence of pancreatic tumor forms a further aspect of the invention. In some cases, the increased phosphorylation may be determined at Threonine 394 of Dual specificity mitogen-activated protein kinase kinase 2. The method may involve determination of increased phosphorylation at this site only, e.g. by immunohistochemistry, or it may include determination at this site in combination with other phosphorylation sites. The method may further include determination of increase or decrease in phosphorylation of sites on one or more further proteins selected from Table 11.

In a further embodiment of this fourth aspect of the invention, the method allows the determination of drug susceptibility for said tumor under test. The inventors have determined from their analysis of the phosphopeptide data that tumors can be classified with respect to the signaling pathways that are affected compared to non-tumor and consequently personalised treatment regimes can be designed based on the drug targets most susceptible in the tumor. In particular, Table 15 provides those proteins (enzymes) which contain phosphorylation sites known to either induce activation or inhibition of the protein (enzyme). Thus, the method may identify a plurality of proteins selected from Table 15 which have been regulated (up- or down-regulated) and thus provide information as to the signalling pathways affected in the tumor. This information allows the clinician to determine a personalised drug treatment regime for said subject by selecting those drugs known to target the particular proteins in said signalling pathways. The drugs may be selected from the group consisting of Dasatinib, Sorafenib, Vorinostat, Temsirolimus, AEZS-131 and GSK2141795.

In a preferred embodiment, the plurality of proteins selected from Table 15 include Tyrosine-protein kinase (Fyn), Tyrosine-protein kinase CSK (Src), RAF proto-oncogene serine/threonine-protein kinase, Histone deacetylase 1, Histone deacetylase 2, Rapamucin-insensitive companion of mTOR (RICTOR); ERK1 mitogen-activated protein kinase, ERK2 mitogen-activated protein kinase, and/or RAC-alpha serine/threonine-protein kinase.

Table 15 and Table 4 provide details of those peptides which contain phosphorylation sites which are known to inhibit or activate the protein when phosphorylated. The proteins containing these sites have been identified by the inventors as being either up or down regulated in tumor as compared to background (normal) tissue. As a result, these sites can be used as markers for pancreatic tumor and depending of which proteins are regulated in the particular sample, can be used to select the drug combination used to treat the subject to inhibit the growth or recurrence of the tumor.

In a still further preferred embodiment, the plurality of proteins is selected from the group consisting of Integrin Beta-4; Catenin alpha-1, Junctional adhesion molecule A (JAM-A); Tyrosine protein kinase Fyn; Mitogen-activated protein kinase 1 (MAPK1); RAC-alpha serine/threonine-protein kinase (AKT1); Glycogen synthase kinase-3 alpha.

The biological sample obtained from said subject is preferably a biopsy sample taken from an individual suspected of having pancreatic cancer. The method may be performed on a number of biopsy samples from said subject over a period of time so as to monitor the effectiveness of the drug treatment.

In a preferred embodiment, the steps of comparing expression levels and/or phosphorylation levels and determining the molecular phenotype of tumour may be carried out using the pancreatic tumour classification system according to the first aspect.

The inventors have used an adapted liquid chromatography-mass spectrometry (LC-MS/MS) method to perform the proteomic analysis of the pancreatic tumor samples. While this may be a preferred method, now that specific biomarkers have been determined by the inventors, i.e. those proteins that are significantly up- or down-regulated in tumor as opposed to non-tumor, other standard methods may be adopted for determining these markers in a sample. Indeed, the inventors have determined a number of markers which are so significantly modulated in tumor tissue that they can act as individual markers thereby avoiding the analysis of multiple markers.

Accordingly, the method of this and other aspects of the invention, for determining the amount of the one or more, or plurality of proteins in the biological sample may be achieved using any suitable method. The determination may involve direct quantification of the protein mass or concentration. The determination may involve indirect quantification, e.g. using an assay that provides a measure that is correlated with the amount (e.g. concentration) of the protein. In certain cases of the method of this and other aspects of the invention, determining the amount of the one or more, or plurality of proteins comprises:
contacting said sample with a specific binding member(s) that selectively and independently binds to the one or more, or plurality of proteins; and
detecting and/or quantifying a complex formed by said specific binding member(s) and the one or more, or plurality of proteins.

The specific binding member may be an antibody or antibody fragment that selectively binds to the protein biomarker. It is preferable that the antibody is labelled for detection. For example, a convenient assay format for determination of a protein concentration is an ELISA. The determination may comprise preparing a standard curve using standards of known concentration for the peptide concentration and comparing the reading obtained with the sample from the subject with the standard curve thereby to derive a measure of the protein biomarker concentration in the sample from the subject. A variety of methods may suitably be employed for determination of protein amount (e.g. concentration), non-limiting examples of which are: Western blot, ELISA (Enzyme-Linked Immunosorbent assay), RIA (Radioimmunoassay), Competitive EIA (Competitive Enzyme Immunoassay), DAS-ELISA (Double Antibody Sandwich-ELISA), liquid immunoarray technology (e.g. Luminex xMAP technology or Becton-Dickinson FACS technology), immunocytochemical or immunohistochemical techniques, techniques based on the use of protein microarrays including reverse protein microarrays and reverse phospho-protein arrays that include specific antibodies, “dipstick” assays, affinity chromatography techniques and ligand binding assays. The specific binding member may be an antibody or antibody fragment that selectively binds a protein biomarker. Any suitable antibody format may be employed. A further class of specific binding members contemplated herein in accordance with any aspect of the present invention comprises aptamers (including nucleic acid aptamers and peptide aptamers). Advantageously, an aptamer directed to the protein biomarker may be provided using a technique such as that known as SELEX (Systematic Evolution of Ligands by Exponential Enrichment), described in U.S. Pat. Nos. 5,475,096 and 5,270,163.

In some cases of the method in accordance with this and other aspects of the invention, the determination of the amount of the protein biomarkers selected from the referenced Tables may comprise measuring the level of a peptide unique to said protein by mass spectrometry. Techniques suitable for measuring the level of a peptides by mass spectrometry are readily available to the skilled person and include techniques related to Selected Reaction Monitoring (SRM) and Multiple Reaction Monitoring (MRM)isotope dilution mass spectrometry including SILAC, AQUA (as disclosed in WO 03/016861; the entire contents of which is specifically incorporated herein by reference) and TMTcalibrator (as disclosed in WO 2008/110581; the entire contents of which is specifically incorporated herein by reference). WO 2008/110581 discloses a method using isobaric mass tags to label separate aliquots of all proteins in a reference sample which can, after labelling, be mixed in quantitative ratios to deliver a standard calibration curve. A patient sample is then labelled with a further independent member of the same set of isobaric mass tags and mixed with the calibration curve. This mixture is then subjected to tandem mass spectrometry and peptides derived from specific proteins can be identified and quantified based on the appearance of unique mass reporter ions released from the isobaric mass tags in the MS/MS spectrum.

By way of a reference level, the marker protein(s) as selected from Table 2, 3, 4, 11, 12, 13 and/or Table 15 may be used. In some cases, when employing mass spectrometry based determination of protein markers, the methods of the invention comprises providing a calibration sample comprising at least two different aliquots comprising the marker peptide(s), each aliquot being of known quantity and wherein said biological sample and each of said aliquots are differentially labelled with one or more isobaric mass labels. Preferably, the isobaric mass labels each comprise a different mass spectrometrically distinct mass marker group.

Accordingly, in a preferred embodiment of the invention, the method comprises determining a change in expression level or phosphorylation level of one or more, or a plurality of the marker proteins selected from Table 2, 3, 4, 11, 12, 13 and/or Table 15 by Selected Reaction Monitoring using one or more determined transitions for the known protein marker derived peptides; comparing the peptide levels in the sample under test with peptide levels previously determined to represent pancreatic cancer based on changes in expression of said one or more, or plurality of marker proteins. The comparison step may include determining the amount of the marker peptides from the sample under test with known amounts of corresponding synthetic peptides. The synthetic peptides are identical in sequence to the peptides obtained from the sample, but may be distinguished by a label such as a tag of a different mass or a heavy isotope.

One or more of these synthetic marker peptides (with or without label) form a further aspect of the present invention. These synthetic peptides may be provided in the form of a kit for the purpose of diagnosing pancreatic cancer in a subject; or for the purpose of classifying a pancreatic sample from a subject into a molecular phenotype selected from tumor, non-tumor, likelihood or recurrence, likelihood of non-recurrence, drug susceptibility, primary tumor, or secondary (metastatic tumor); or for selecting a treatment regimen for said subject.

In preferred embodiments with respect to this and other aspects of the invention, the one or more proteins, or plurality of proteins includes Mucin-1 and/or Homeodomain-interacting protein kinase-1; optionally in combination with one, two, three or four further proteins selected from Table 2, 3, 4, 11, 12, 13 and/or 15, preferably Table 12 and/or Table 2.

Other suitable methods for determining levels of protein expression include surface-enhanced laser desorption ionization-time of flight (SELDI-TOF) mass spectrometry; matrix assisted laser desorption ionization-time of flight (MALDI-TOF) mass spectrometry, including LS/MS/MS; electrospray ionization (ESI) mass spectrometry; as well as the preferred SRM and TMT-SRM.

In a further aspect of the invention, there is provided a kit for use in carrying out the methods described above, in particular classifying pancreatic cancer into molecular phenotypes selected from the group consisting of tumor, non-tumor, recurrence, non-recurrence, drug susceptibility, primary tumour and/or secondary (metastatic) tumor for a sample obtained from a subject.

In all embodiments, the kit allows the user to determine the presence, level (up- or down-regulation) of protein expression and/or phosphorylation status of a plurality of analytes selected from a plurality of marker proteins or fragments thereof provided in Table 2, 3, 4, 11, 12, 13 and/or 15 and antibodies against said marker proteins in a sample under test; the kit comprising
(a) a solid support having a plurality of binding members, each being independently specific for one of said plurality of analytes immobilised thereon;
(b) a developing agent comprising a label; and, optionally
(c) one or more components selected from the group consisting of washing solutions, diluents and buffers.

The binding members may be as described above.

In one embodiment, the kit may provide the analyte in an assay-compatible format. As mentioned above, various assays are known in the art for determining the presence or amount of a protein, antibody or nucleic acid molecule in a sample. Various suitable assays are described below in more detail and each form embodiments of the invention.

The kit may additionally provide a standard or reference which provides a quantitative measure by which determination of an expression level of one or more marker proteins can be compared. The standard may indicate the levels of the two or more biomarkers which indicate pancreatic cancer.

The kit may also comprise printed instructions for performing the method.

In a preferred embodiment, the kit may be for performance of a mass spectrometry assay and may comprise a set of reference peptides derived from proteins set out in Table 2, 3, 4, 11, 12, 13 and/or 15 (e.g. SRM peptides) in an assay compatible format wherein each peptide in the set is uniquely representative of each of the plurality of marker proteins. Preferably two, three, four or five or more such unique peptides are used for each protein for which the kit is designed, and wherein each set of unique peptides are provided in known amounts which reflect the levels of such proteins in a standard preparation of said sample. Optionally the SRM peptides are phosphopeptides representing differentially phosphorylated sites within the target proteins set out in Table 13, 3, 11 and/or Table 14. Optionally the kit may also provide protocols and reagents for the isolation and extraction of proteins from said sample, a purified preparation of a proteolytic enzyme such as trypsin and a detailed protocol of the method including details of the precursor mass and specific transitions to be monitored. The peptides may be synthetic peptides and may comprise one or more heavy isotopes of carbon, nitrogen, oxygen and/or hydrogen.

The classification methods as provided herein also include determination of protein modulation as a result of phosphorylation. The inventors have shown that a number of proteins are induced or inhibited in pancreatic cancer tissue as opposed to background tissue. Accordingly, the invention provides a method comprising determining the phosphorylation status of one or more, or a plurality of proteins selected from Table 13, 3, 11 and/or Table 14 in a sample obtained from a subject suspected of having pancreatic cancer.

Preferably said one or more or plurality of proteins are selected from the group consisting of integrin beta-4, Catenin alpha-1, Junctional adhesion molecule A (JAM-A), Tyrosine protein kinase Fyn; Mitogen-activated protein kinase 1 (MAPK1); RAC-alpha serine/threonine-protein kinase (AKT1); Glycogen synthase kinase-3 alpha.

In a preferred embodiment, the protein is Dual specificity mitogen-activated protein kinase kinase 2. In particular, the inventors have determined that phosphorylation of Dual specificity mitogen-activated protein kinase kinase 2 at phospho-T394 was increased in tumor cases compared to background (non-tumor) and have shown that phosphorylation at this site correlates positively with recurrence of tumor at median 16.5 months (FIG. 4).

Table 11, 15 and/or Table 4 provide a list of other phosphorylation sites on proteins which are regulated in pancreatic tumor samples as compared to non-tumor. Each of these sites provides a marker for classifying pancreatic tumor with respect to likelihood of recurrence and drug susceptibility. Accordingly, each phosphorylation site forms an aspect of the present invention either alone or in combination for use in classifying pancreatic tumor with respect to likelihood and timing of recurrence and/or drug susceptibility.

By way of example, there is provided a method of predicting susceptibility of a pancreatic tumor to treatment with Dasatinib (BMS-354825—Sprycel™) comprising determining the level of phospho-S21 on Tyrosine-protein kinase Fyn, wherein an up-regulation of this protein is indicative that the pancreatic tumor will be susceptible to treatment with Dasatinib (Table 4).

Further there is provided a method of predicting susceptibility of a pancreatic tumor to treatment with AEZS-131 (Aeterna Zentaris Inc) and/or SCH772984 (Merck) comprising determining the level of phospho-T185 and/or phospho-Y187 on Mitogen-activated protein kinase 1 (MAPK1); and additionally or alternatively phospho-T202 and/or phospho-Y204 of Mitogen-activated protein kinase 3 (MAPK3/ERK1), wherein an up-regulation of this protein phosphorylation is indicative that the pancreatic tumor will be susceptible to treatment with AEZS-131 and/or SCH772984. For further examples, see Table 4.

Determining phosphorylation of proteins is standard in the art. For example, antibodies that have specificity for a particular phosphorylation motif can be raised in a host animal and used for subsequent detection of the relevant motif in tissues in situ using immunohistochemistry or following extraction of the target protein from the tissue or body fluid using Western blotting or enzyme-linked immunosorbent assay (ELISA). Other antibody-based detection methods are well known to the skilled practitioner and include bead-suspension arrays, planar arrays, radio-immunoassays and immunoprecipitation linked to mass spectrometry. However, it is normally necessary to use phosphoprotein specific antibodies in a two-step process where the target protein is first enriched prior to detection. This is due to the commonality of epitopes recognised by such antibodies within multiple substrates of a particular kinase. In other words, the way a kinase recognises phosphorylation sites within its substrates is similar to the epitope recognised by an antibody being a conserved sequence of 4-8 amino acids.

In some cases phosphorylation of proteins can be monitored by providing a radioactive isotope of phosphorous, typically P32 in a growth medium or dietary supplement for experimental animals. After a defined period of metabolic labelling the incorporation of P32 in specific proteins can be followed by detection the radioactive signal using standard protein separation methods such as gel electrophoresis and liquid chromatography.

In a preferred embodiment, the plurality of proteins selected from Table 13, Table 3, and/or Table 11 include Integrin Beta-4; Catenin alpha-1, Junctional adhesion molecule A (JAM-A); Tyrosine protein kinase Fyn; Mitogen-activated protein kinase 1 (MAPK1); RAC-alpha serine/threonine-protein kinase (AKT1); Glycogen synthase kinase-3 alpha.

In a further aspect of the invention, a method is provided for classifying a pancreatic tumor sample into one or more molecular phenotypes comprising
(1) determining the protein expression levels of one or more, or a plurality of proteins selected from Table 12 and/or Table 2, for both a pancreatic tumor sample and a pancreatic non-tumor sample taken from a subject
and/or
(2) determining the up or down regulation of one or more, or a plurality of phosphoproteins selected from Table 3, Table 13 and/or Table 11 in a pancreatic tumor sample and a pancreatic non-tumor sample taken from a subject,
(3) comparing said protein expression levels of the tumor sample with the non-tumor sample; and/or comparing the up or down regulation of phosphoproteins in the tumor sample with the non-tumor sample
(4) applying predictive algorithm

log₂(ᵢT/ᵢNT)
(where i is subject sample, T=tumour and NT=non-tumour)
to produce a prediction value that for said protein expression level and/or phosphoprotein level for said subject;
(5) classifying said pancreatic tumor sample into a molecular phenotype by reference to a database comprising values predictive of said phenotypes, wherein said database comprises predictive values for one or more or a plurality of proteins selected from Table 2, 3, 4, 11, 12, 13 and/or 15; and wherein the molecular phenotype is selected from tumor, non-tumor; tumor recurrence, tumor non-recurrence; drug susceptibility; primary and/or secondary tumor.

In a preferred embodiment the protein marker is considered modulated (either by up-regulated or down-regulated expression or phosphorylation) if the log₂ T/NT ratio is ≧1 or ≦−1.

In a preferred embodiment, the classification is carried out by a pancreatic tumor classification system according to the first aspect.

Preferably the above method may be used to determine the prognosis of a subject with pancreatic cancer. In this respect, prognosis includes the determination or early, late or no recurrence following surgical removal, radiological or chemotherapy treatment. For example the method may compare the expression and phosphorylation values with values for one or more or a plurality of proteins selected from Tables 11, 3, and/or 13.

In preferred embodiment, the one or more or plurality of proteins includes Dual specificity mitogen-activated protein kinase kinase 2.

In respect of this and other aspects of the invention, the total protein content of a surgically-resected tumor or a tumor biopsy is extracted and subjected to phosphoproteomic analysis by methods known in the art and/or described herein. The relative abundance of each phosphopeptide detected by such analysis is recorded in a database (e.g. using a system according to the first aspect) and the total profile is compared with known cases of recurrent and non-recurrent pancreatic cancer using methods such as Agglomerative Clustering. By this “bottom up” approach: each observation starts in its own cluster, and pairs of clusters are merged as one moves up the hierarchy. At the end of the Agglomerative Clustering process the tumor being analysed will have been clustered into a group representing its likelihood of recurrence. In a preferred embodiment, the database also carries sufficient numbers of samples with specific times of recurrence post-surgery or initial treatment to also assign a likely time of recurrence to the individual patient with a recurrent tumor profile. The likely time of recurrence is between 8 and 33 months, between 10 and 20 months or between 15 and 17 months after removal of the tumor.

In a further aspect of the invention, there is provided a method selecting a treatment regimen for a subject with pancreatic cancer, said method comprising
(1) determining phosphoprotein levels of one or more, or a plurality of protein markers selected from Table 15 and/or Table 4,
(2) comparing said determination with a previously determined reference representative of drug susceptibility, and
(3) selecting a drug treatment regime for said subject based on the drug susceptibility of said tumor.

In a preferred embodiment, the drug target is a particular protein carrying a differential phosphorylation site, or it is an upstream kinase or phosphatase responsible for such differential phosphorylation.

In a preferred embodiment, the plurality of proteins selected from Table 15 and/or Table 4 include Tyrosine-protein kinase Fyn, Tyrosine-protein kinase CSK (Src), RAF proto-oncogene serine/threonine-protein kinase, Histone deacetylase 1, Histone deacetylase 2, Rapamucin-insensitive companion of mTOR (RICTOR); ERK1 mitogen-activated protein kinase, ERK2 mitogen-activated protein kinase, and/or RAC-alpha serine/threonine-protein kinase.

Preferably the drugs are selected from the group consisting of Dasatinib, Sorafenib, Vorinostat, Temsirolimus, AEZS-131 and GSK2141795.

For all aspects of the invention, the determination step is preferably carried out by liquid chromatography-mass spectrometry (LC-MS/MS).

In a still further aspect of the invention a method for improving the design of molecular targeting drugs is provided wherein the methods and systems of the invention are used to analyse the performance of novel compounds in modulating the oncogenic pathway on the proteins selected from Tables 2, 3, 12, 11, 13, 14 and/or 15.

Accordingly, the invention further provides a method of testing the effectiveness of a molecular targeting drug comprising
obtaining a sample of pancreatic tumor from a subject; said tumor having been in contact with the molecular targeting drug under test, e.g. by administration to said subject prior to the sample being obtained;
extracting proteomic data from said sample, e.g. relative abundance of proteins or phosphorylated proteins;
comparing said proteomic data with reference data, e.g. data obtained from a sample of the same tumor prior to contact with the molecular targeting drug under test;
wherein a change in the proteomic data between the sample taken after contact with the molecular targeting drug and the sample taken prior to contact with the molecular targeting drug is indicative of the effectiveness of the molecular targeting drug in treating pancreatic tumor; and
wherein the proteomic data comprises relative abundance levels of a plurality of phosphoproteins selected from Table 15 and/or Table 4.

The proteomic data may be obtained by measuring the relative abundance (e.g. up-regulated or down-regulated) of phosphopeptides unique to each of the plurality of proteins. Preferably the phosphopeptides are selected from Table 15 and/or Table 4.

By way of example, human pancreatic cancer-derived cell lines are exposed to a candidate therapeutic compound at different concentrations, including a vehicle control, or for different periods of time. Following exposure to the candidate therapeutic compound, cells are lysed and total proteins extracted. Preferably the proteins are digested using a proteolytic enzyme such as trypsin and labelled, e.g. using an isobaric mass tag. Preferably the isobaric mass tags are Tandem Mass Tags (Thermo Scientific). Labelled peptides from several cell lines may be mixed together prior to analysis by LC-MS/MS. Preferably one or more reference labelled peptides (e.g. selected from Table 15 and/or Table 4) representing known targets of the candidate drugs may be included to provide a quantitative internal standard. Following LC-MS/MS analysis the relative abundance of one or more, and preferably all phosphopeptides in each treated sample are submitted to analysis in a system according to the first aspect e.g. the SysQuant database, and subjected to Agglomerative Heirarchical Clustering to obtain a treatment phenotype. Compounds achieving a positive treatment phenotype may be prioritised for further development.

It is to be understood that the methods of this aspect of the invention may be applied to any aspect of the drug development process including xenograft tumors and tumors taken from human subjects participating in clinical trials.

It is further to be understood that the methods of this aspect of the invention may also be applied to the determination of the most effective molecular targeting medicines in a patient with a pancreatic tumor based on preparation of primary tumour cell cultures from the resected tumor, exposure of primary cell cultures to different molecular targeting drugs and analysis of the relative levels of phosphoproteins using the methods described herein, e.g. inventors' SysQuant methods.

Preferably the proteins (or their unique peptides) include one or more of, or a plurality of, Tyrosine-protein kinase Fyn, Tyrosine-protein kinase CSK (Src), RAF proto-oncogene serine/threonine-protein kinase, Histone deacetylase 1, Histone deacetylase 2, Rapamucin-insensitive companion of mTOR (RICTOR); ERK1 mitogen-activated protein kinase, ERK2 mitogen-activated protein kinase, Intergrin beta 4, Catenin alpha-1, Junctional adhesion molecule A (JAM-A); Mitogen-activated protein kinase 1 (MAPK1); Glycogen synthase kinase-3 alpha; Homeodomain-interacting protein kinase (HIPK1); Serine/threonine-protein kinase MRCK alpha (MRCK alpha); Myosin light chair kinase, smooth muscle (MLCK) and/or RAC-alpha serine/threonine-protein kinase (AKT1).

In a further aspect of the invention the methods and systems of the invention e.g. the SysQuant database, may be applied to the analysis of recurrent pancreatic cancer. When a new tumour is identified in a patient that has previously received treatment for pancreatic cancer, a so-called recurrent tumor, or a new tumor is found in the pancreas of patients that have previously been treated for a tumor elsewhere in the body, a so-called metastatic tumor, it is important to identify the mechanism of resistance and potential new targets for treatment in the recurrent or metastatic tumor. Accordingly, the methods of the present invention may be utilised in the analysis of protein and phosphorylation site changes in the recurrent or metastatic tumor.

The invention also provides the use of a plurality of biomarkers selected from Table 2, 3, 4, 11, 12, 13 and/or 15 for determining the molecular phenotype of a pancreatic tumor in a subject, wherein said molecular phenotype is selected from the group consisting of tumor, non-tumor, recurrence, non-recurrence, drug susceptibility, primary tumour and/or secondary (metastatic) tumor.

Preferably the biomarkers are selected from Table 2 and/or Table 12 and the molecular phenotype is selected from tumor or non-tumor.

In particular, the biomarkers may comprise Mucin-1, Intergrin beta 4, and/or Homeodomain-interacting protein kinase 1.

In a further embodiment, the biomarkers are selected from Table 3, 11 and/or Table 13 and the molecular phenotype is selected from tumor recurrence or tumor non-recurrence, e.g. Dual specificity mitogen-activated protein kinase kinase 2.

In a still further embodiment, the biomarkers are selected from Table 4 and/or 15 and the molecular phenotype is selected from drug susceptibility. For example, the biomarkers may include one or more of, or a plurality of, Tyrosine-protein kinase Fyn, Tyrosine-protein kinase CSK (Src), RAF proto-oncogene serine/threonine-protein kinase, Histone deacetylase 1, Histone deacetylase 2, Rapamucin-insensitive companion of mTOR (RICTOR); ERK1 mitogen-activated protein kinase, ERK2 mitogen-activated protein kinase, Intergrin beta 4, Catenin alpha-1, Junctional adhesion molecule A (JAM-A); Mitogen-activated protein kinase 1 (MAPK1); Glycogen synthase kinase-3 alpha; and/or RAC-alpha serine/threonine-protein kinase (AKT1).

The inventors have determined a number of protein kinases which are consistently differentially expressed in tumor versus non-tumor patients. Accordingly, the invention provides a number of novel therapeutic targets for pancreatic cancer. In addition, the invention provides methods of treating subjects with pancreatic cancer using kinases inhibitors. In one embodiment, the invention provides a method of treating pancreatic cancer in a subject, said method comprising administering a compound effective in inhibiting the kinase activity of one or more proteins selected from HIPK1; MRCK alpha; and MLCK.

Certain aspects and embodiments of the invention will now be illustrated by way of example and with reference to the figures and tables described above. The present invention includes the combination of the aspects and preferred features described except where such a combination is clearly impermissible or is stated to be expressly avoided. All documents mentioned in this specification are incorporated herein by reference in their entirety for all purposes.

### BRIEF DESCRIPTION OF THE FIGURES AND TABLES

FIG. 1 Venn diagrams demonstrate the number of; A. unique phosphopeptides, B. unique non-phosphopeptides, and C. unique total peptides identified in the TiO2, IMAC, and/or non-enrich arm of the SysQuant workflow, across all three TMT8plex samples in total (TMT8plex-ALL) and individually per TMT8plex (TMT8plex 1, TMT8plex 2, TMT8plex 3). 1.D demonstrates the level of overlap the inventors observed for peptide identifications from analytical run 1, analytical run 2, and analytical run 3 (including time dependent rejection list compiled from identifications from run 1 and 2).

FIG. 2A: PC1 and PC2 Score plot of the first two principal components describing 13.6% (PC1) and 10.6% (PC2) of the total variance in the data. The circle depicts the T2 hotelling space based on 95% confidence. 2B: PC2 and PC3 Score plot of the next principal components describing 10.6% (PC2) and 14.4% (PC3) of the total variance in the data.

FIG. 3: Hierarchal cluster analysis was performed on log₂ T/NT values of all 5409 phosphopeptides quantified in this study. Phosphopeptides are clustered in rows and cases are clustered in columns. 3A: focusses on regions of the cluster map which contain phosphopeptides demonstrating lower levels (GREEN) in tumor tissue from patients with recurrence, but higher levels (RED) in tumor from patients with no recurrence. The red arrows indicate phosphopeptides that correlate best with recurrence. 3B: focusses on regions of the cluster map which contain phosphopeptides demonstrating the inverse of 3A. 3C: phosphopeptides demonstrating lower levels in tumor from all cases (upper panel), and higher levels in tumor from all cases (lower panel). 3D: Pearson's correlation coefficients were calculated across all cases and hierarchal clustering was performed on these values. The table indicates presence or absence of lymph node metastases and recurrence in each case.

FIG. 4. All log 2 T/NT ratios of phospho-peptides containing phospho-T394 of Dual specificity mitogen-activated protein kinase kinase 2 were summed and displayed on the table and plotted on the bar chart. Patients with recurrence (median 16.5 month follow up) were grouped with patients with no recurrence at time of last examination. Time of examination, time of recurrence, time of tissue storage in −80° C. freezer and presence of lymph node mets are displayed in the table.

FIG. 5. Volcano plots showing −log₁₀ P-values in relation to log₂ T/NT ratios for; (A) proteins and (B) phosphopeptides measured in the IMAC, (C) phosphopeptides measured in the TiO₂ and (D) phosphopeptides measured in the Non-enriched arm of the SysQuant workflow. Red circles point out biologically significantly phosphopeptides as they demonstrate log₂ T/NT ratios ≧0.75 or ≦−0.75 and have p-values ≦0.05. E: is a Venn diagram illustrating the distribution of the 635 phosphopeptides across the three arms of the workflow that were significantly modulated.

FIG. 6.A: shows a STRING protein interaction network built using accession numbers from all proteins with significantly regulated phosphopeptides. In total there were 635 significantly modulated phosphopeptides from 408 proteins in the illustrated network. B: shows the same STRING network but highlights in RED those proteins involved in the KEGG Tight Junction signaling pathway. The phosphopeptides from the Tight Junction proteins are also listed. C: highlights in RED those proteins associated to the GO biological process ‘Regulation of RAS protein signal transduction’ and there phosphopeptides are listed in the table.

FIG. 7. Signaling pathways modulated in pancreatic cancer tissue. (A) This schema summarizes all proteins identified as phosphorylated from the following KEGG signaling pathways; Tight Junction, Adherens Junction and Focal Adhesion. Red stars indicate those proteins identified as phosphorylated in any of 12 cases. Proteins highlighted by coloured circles are known drug targets. (B) Phosphopeptides from case 1 (FIG. 4B) demonstrating log₂ T/NT ratios ≧1 or ≦−1, were from proteins matched with greatest significance (based on Benjamini) by the DAVID Bio-informatic resource to the Tight Junction and Adherens Juntion signaling pathways from KEGG. Red stars indicate proteins yielding phosphopeptides with log₂ T/NT ratios ≧1 or ≦−1 from case 1, and coloured circles indicate most suitable drug target, which in case 1 is FYN. (C) Phosphopeptides from case 10 demonstrating log₂ T/NT ratios ≧1 or ≦−1, were from proteins matched with greatest significance (based on Benjamini) by the DAVID Bio-informatic resource to the Tight Junction and Focal Adhesion signaling pathways from KEGG. Red stars indicate proteins yielding phosphopeptides with log₂ T/NT ratios ≧1 or ≦−1 from case 10, and coloured circles indicate most suitable drug target, which in case 10 appears to be AKT1 and MAPK1.

FIG. 8A: This MA-plot shows the logarithmized ratios vs. the logarithmized intensities over the complete non-normalized data set. FIG. 8B: This MA-plot shows the same as FIG. 8A, but the data are normalized by sum-scaling and therefore better zero-centred.

Table 1: Number of peptide spectrum matches, number of unique peptides and number of phosphorylation sites identified in each TMT8plex and in total.

Table 2: Top 12 proteins significantly up-regulated in tumor compared to background tissue, on average over all 12 cases. Log2 T/NT ratios of the non-phosphorylated peptides from each protein were used as surrogates to calculate the relative abundance of the respective proteins. Log2 T/NT ratios of the non-phosphorylated peptides were averaged over three arms of the workflow (IMAC, TiO₂, Non-enrich).

Table 3: Significantly regulated phosphopeptides in tumor compared to background tissue, on average over all 12 cases. All phosphopeptides are from proteins involved in KEGG signaling pathways; Tight Junction, Focal Adhesion, Vascular Smooth Muscle Contraction, Rearrangement of Actin Cytoskeleton. Here we display the p values and Log2 T/NT ratios for protein and phosphopeptide.

Table 4: Displays examples of peptides that contain activator and inhibitor phosphorylation sites on proteins known to be anti-cancer drug targets. The phosphorylated residue in each peptide sequence is underlined. The log 2 T/NT ratios were median values calculated from all three arms of the workflow, and all ratios ≧1 or ≦−1 were highlighted in bold text. Peptides in red contain activator phosphorylation sites, while peptides in blue contain inhibitor phosphorylation sites. Peptides in black contain phosphorylation sites with no known function.

Table 5: Characteristics of fourteen cases of pancreatic head ductal adenocarcinoma were selected from Institute of Liver Studies BioBank for use in this study.

Table 6: Tumor stage and recurrence of each case under study. Yellow cases showed recurrence between 8 & 33 months (median follow-up period 16.5 months) after tumor removal. The difference between stage IIA and IIB is the presence (IIB) or absence (IIA) of lymph node metastasis.

Table 7: Clinical information (e.g. time of recurrence) for each case under test.

Table 8: Protein amounts from each sample used for the SysQuant workflow in this study.

Table 9: Peptides are labelled with different tandem mass tags (TMT). Table 9 shows which TMT8plex tag is used to label which sample within each of the three TMT8plex samples analysed in this study.

Table 10: All three of the TMT8plex samples were separated into 3 aliquots. All nine aliquots of TMT labelled peptides were then separated by SCX-HPLC into 12 fractions each. For each of the three TMT8plex samples, 12 fractions were enriched for phosphopeptides using IMAC, 12 fractions enriched for phosphopeptides using TiO2, and 12 fractions were not enriched for phosphopeptides but instead analysed directly by LC-MS/MS to determine relative protein abundance for normalisation purposes.

Table 11: Phosphopeptides displaying high (Log2 T/NT 0.7) and low (log 2 T/NT≦−0.7) levels in tumour versus non-tumor from the cases with recurrence that clustered together in FIG. 3D.

Table 12: Significantly regulated proteins in tumor versus non-tumour (150 proteins). T.test p-values and average log 2 T/NT ratios across 12 cases as well as Log2 T/NT ratios for each case are provided.

Table 13: Accession numbers of proteins involved in signaling pathways (Kegg pathways shown in column entitled ‘term’) which also yielded phosphopeptides demonstrating log 2 T/NT ratios of ≧1, or ≦−1 (more than 2 fold up/down-regulated) from each case. Information such as p values and Benjamini probabilities are also shown.

Table 14: Case 1—Phosphopeptides from case 1 displaying log 2 T/NT ratios ≧1 or ≦−1, from proteins involved in the following KEGG signaling pathways Tight Junction, Adherens Junction and Focal Adhesion

Table 15: Case by case—Phosphopeptides displaying log 2 T/NT ratios ≧1 or ≦−1 at sites known to either induce activation or inhibition of the phosphorylated enzyme.

### ABBREVIATIONS AND DEFINITIONS

HPLC=high pressure liquid chromatography
SCX=strong cation exchange
TiO₂=titanium dioxide
IMAC=immobilised metal affinity chromatography
T=tumor
NT=non-tumor
LC-MS=liquid chromatography—mass spectrometry
STRING=Search Tool for the Retrieval of Interacting Genes/Proteins
GO=Gene Ontology
KEGG=Kyoto Encyclopedia of Genes and Genomes
TMT=Tandem mass tags

The phenotype “tumor” in the context of the present invention shall mean neoplastic cells resulting in abnormal proliferation (malignant growth) as a result of carcinoma of the pancreas, in particular pancreatic head adenocarcinoma.

The phenotype “non-tumor” in the context of the present invention shall mean normal, non-neoplastic or benign neoplastic pancreatic cells. It will be understood that such cells may be obtained from abnormal growth, but such growth is not malignant, e.g. cyst.

The phenotype “likelihood of recurrence” shall mean the likelihood of the tumor reappearing between 8 and 30 months following removal by e.g. surgery.

The phenotype “likelihood of non-recurrence” shall mean the likelihood of the tumor not reappearing following removal by e.g. surgery.

The phenotype “drug susceptibility” in the context of the present invention shall mean a pancreatic tumor presenting a molecular profile indicative of modulation of a cell signalling pathway comprising one or more molecular drug targets. The drug targets may be selected from FYN, GSK3α/β, HDAC1/2, the RAF kinases, MAPKs (p38 and ERK2), AKT, PKCs, Casein Kinases.

The phenotype “primary tumor” shall mean tumor originating from the pancreas.

The phenotype “secondary tumor” or “metastatic tumor”, shall mean a pancreatic tumor that is formed by cancer cells originating from a tumor located elsewhere in the subject.

The term “plurality” may mean more than one, more than two, more than three, more than four, more than five, more than 10, more than 15, more than 20, more than 25, more than 30 proteins, peptides, phosphoproteins or phosphopeptides selected from one or more referenced Table.

The term “plurality” may also mean more than one protein, peptide, phosphoprotein, phosphopeptide as expressed as a percentage of the reference Table. For example, a plurality may include 10%, 20%, 30%, 40% 50%, 60%, 70%, 80%, 85%, 90%, 95% of the proteins, peptides, phosphoproteins or phosphopeptides provided in the referenced Table.

In both cases, where the plurality is selected from a referenced Table, it is envisaged that any combination of the proteins, peptides, phosphoproteins, or phosphopeptides will form embodiments of the present invention. For example, with respect to Table 2 where 12 proteins are listed, it is contemplated that the plurality of proteins may comprise Homeodomain-interacting protein kinase 1 with one or more, two or more, three or more etc of the remaining proteins listed in Table 2. This would be true for each of the proteins independently, i.e. Mucin-1 may be combined with one or more, two or more, three or more etc of the remaining proteins listed in Table 2.

By way of example, such combinations can be expressed mathematically notation “combination”:

$$( \begin{matrix}n \\ k\end{matrix} ) = \frac{nl}{kl ( n - k ) l}$$

This can be expressed in the form ⁿCₖ (i.e. “n choose k”) In the case of Table 12, n=12 (the total of the table) and k is the number in a chosen subset.

All combinations of two or more markers from Tables 2, 3, 4, 11, 12, 13, 14 and/or 15 are specifically contemplated herein, i.e. for

Table 2 all 66 possible pairs (¹²C₂), all 220 possible combinations of 3 markers (¹²C₃), all 495 possible combinations of 4 markers (¹²C₄), all 792 possible combinations of 5 markers (¹²C₅), all 924 possible combinations of 6 markers (¹²C₆), etc.

The term “protein” shall be construed to include the full length protein or any form of the protein, e.g. translational splice variants, isoforms, glycosylated forms, phosphorylated forms or comprising other post-translational modifications. For the proteins referenced in the Tables, Uniprot-IDs are provided allowing full details of the protein including its sequence to be obtained. It is understood in the art that each Uniprot-ID has a history log that allows the specific sequence associated with said Uniprot-ID on any given date such as the date of the present invention can be readily determined irrespective of subsequent modification or revision. This information and data is incorporated herein by reference.

Accordingly, a change in expression level of a protein may mean the up- or down-regulation of the expression of the protein in all its forms, or it may mean the up- or down-regulation of a particular form of the protein, e.g. isoform, splice variant etc.

The term “relative abundance” shall mean the level, amount or concentration of a protein as compared to a reference level, i.e. from a database or from levels obtained from a different/background sample. The relative abundance of a protein may be obtained from measuring the level, amount or concentration of one or more, preferably two, three, four or five peptides unique to said protein and comparing the level, amount or concentration with the same peptides in the reference sample. This provides relative abundance levels for each peptide. A median average may then be taken to illustrate the level, amount or concentration of the protein itself.

The term “peptide” shall mean an amino acid sequence derived from a full length protein. The peptide will comprise enough amino acids such that its sequence is unique to the protein from which it is derived. This may be as few as at least 4, 5, 6, 7, 8, 9 or 10 amino acids in length, more preferable between 4 and 50, 40, 35, 30, 25 or 20 amino acids, or between 5 and 50, 45, 40, 35, 30, 25 or 20 amino acids or between 5 and 50, 45, 40, 35, 30, 25, or 20 amino acids. The peptide may be made synthetically, or it may be the result of proteolytic enzyme digestion, e.g. trypsin of the full length protein.

The term “phosphoprotein” shall mean any protein which has been phosphorylated at a phosphorylation site e.g. serine, tyrosine or threonine. Herein, such sites are denoted as ‘phospho-Xyyy’ where X represents the one or three letter amino acid code and y represents integers defining the residue location within the Uniprot-ID of the relevant phosphoprotein.

The term “phosphopeptide” shall mean a peptide sequence which comprises one or more, preferably one, phosphorylated site, e.g. serine, tyrosine or threonine.

A change in the level or phosphorylation status of a phosphoprotein or phosphopeptide derived from a phosphoprotein does not necessarily mean a change in the amount (concentration) of the protein itself, but rather a change in the phosphorylated form of said protein, perhaps at a specific site.

### Materials and Methods

Twelve cases of pancreatic head ductal adenocarcinoma were selected (Table 5). Case selection is described in Supplemental methods below. Briefly, 12 tumor (T) versus 12 non-tumor (NT) pancreatic tissue specimens were analysed using the SysQuant workflow. Tissue samples were taken from the pancreatic tumor masses, while NT samples were taken from the same pancreas at a distal site from the tumor mass. All tissue samples were frozen within 30 minutes of surgical resection and stored at −80° C. until analysis by SysQuant (median time of storage 18.5 months (range 4-28 months)). Details of experiments are described in Supplemental Methods below. In summary, this entailed protein extraction from tissue specimens, trypsin digestion of proteins into peptides, TMT 8-plex labelling of peptides (tumor and non-tumor tissue from 4 cases per TMT 8-plex) followed by mixing to form a single 8-plex sample mixture (See Table 9). Each TMT 8-plex sample was then split into three independent aliquots, each of which was further split into 12 fractions by strong cation exchange (SCX) chromatography (Table 10). The first set of 12 SCX fractions were then analysed directly by nano-flow HPLC-MS/MS using duplicate data dependent acquisition runs followed by a third run using time dependent rejection of all features identified in runs 1 & 2. The remaining two sets of 12 fractions were first enriched for phosphopeptides using either IMAC or TiO₂ (Table 10). The resulting 24 phosphopeptide enriched fractions were submitted to the same nano-flow HPLC-MS/MS analysis. In total 108 separate nano-flow HPLC-MS/MS runs were performed for each TMT 8-plex sample. Raw MS data were searched against the human UniProtKB/Swiss-Prot database using Mascot and Sequest (via Proteome Discoverer). Peptide spectrum matches (PSMs) were rejected if they were identified with only low confidence 5% FDR), showed 75% phospho-RS probability score, and had missing quantification channels (e.g. not all peaks for isobaric tags were visible in spectra). Raw intensity values of isobaric tags from PSMs passing filters were used for quantification, and these values were normalised using sum-scaling to reduce potential experimental/systematic bias. As a first step, log₂ ratios were calculated from isobaric tag intensities, showing the regulation between T over NT for all and for each case. A phosphopeptide T/NT log₂ ratio is the median T/NT log₂ ratio from all PSMs unique to that specific peptide sequence. A protein T/NT log₂ ratio is the median T/NT log₂ ratio from all unique non-phosphorylated peptides unique to that specific protein. For the data analysis a one sided t-test (one-sample location test) was used to calculate p-values. P-values were plotted against log₂ T/NT ratios on Volcano plots to detect any significant regulation over all cases. At the protein level, annotation using GO-terms (http://www.geneontology.org/), KEGG-pathways (http://www.genome.jp/kegg/) and Drugbank (http://www.drugbank.ca/) information were added, and also mapped to pathways using resources such as DAVID (http://david.abcc.ncifcrg.gov/) and STRING (http://string-db.org/). At the phosphorylation site level annotation using PhosphoSitePlus (www.phosphosite.org) were added, including known functional and biological/pathological role of the phosphorylation site. Principal component analysis (PCA) and Projection to Latent Structure (PLS) were used to model/investigate the multivariate dataset and identify outliers and groups/clusters, from all peptide ratios (phospho and non-phospho peptides) from all arms of the workflow (IMAC, TiO₂ and non-enriched). Finally hierarchal clustering were performed to build a hierarchy of clusters at the case/specimen level in relation to phosphopeptide relative abundance between T and NT tissue types, and also in relation to the protein relative abundance. The SysQuant workflow, combining phosphoproteomic sample preparation, LC-MS/MS analysis, and bioinformatics analysis, was used to identify important molecular events the inventors believe contribute to pancreatic cancer in the cases analysed here.

### Supplemental Methods

### Frozen Clinical Tissue.

Ethical aspects and research protocol were approved by the BioBank Committee of the Institute of Liver Studies, King's College Hospital. Twelve cases of pancreatic head ductal adenocarcinoma were selected in the database of BioBank at the Institute of Liver Studies (Table 5). Initially cases 2 and 3 were selected but later found to have too little protein extracted for this workflow. Therefore two additional cases were selected (Cases 13 and 14) to increase the number back to twelve. Small pieces of tissue were snap frozen from Whipple's specimens and stored in a BioBank freezer (for at least 2 years). This process of tissue sampling was completed within 30 min. Paired samples of cancer (tumor) and background (non-tumor) were used for each case. Table 6 describes tumor grade and whether recurrence was present at median follow up of 16.5 months (Range between 8 & 33 months).

### Tissue Cell Lysis.

Frozen clinical tissue samples were pulverized then ground into a fine powder using a Pestle and Mortar in the presence of liquid nitrogen. The powder was then transferred to eppendorf tubes containing 1.3 mL of ice cold lysis buffer (8M urea, 75 mM NaCl, 50 mM Tris-pH 8.2, one tablet of protease inhibitors cocktail (complete mini, Roche) per 10 mL of lysis buffer, and one tablet of phosphatase inhibitor cocktail (Roche) per 10 mL of lysis buffer). Samples were then sonicated at 20% Amplitude for 20×1 second, pulsing on and off, on ice (4° C.). Following centrifugation at 12,500 g for 10 min at 4° C., the protein concentration of each sample were then determined using the Bradford protein assay and microplate luminometer. Protein amounts used for this workflow for each TMT 8-plex are shown in Table 7.

### In-Solution Trypsin Digestion.

Reduction, alkylation of cysteines, and digestion was performed on lysates by following the Villen and Gygi, Nature Protocol, approach

[Villen, J., Gygi S. The SCX/IMAC enrichment approach for global phosphorylation analysis by mass spectrometry. Nature Protocols. 3, 1630 (2008)]. The digested samples were spun for 10 minutes at 2,500 g and de-salted on 100 mg SepPak tC18 cartridges (Waters, Milford, Mass., USA). Peptides were eluted with 50% ACN/0.1% TFA and lyophilised.

### TMT Labelling.

Digested peptides from all samples were separately re-suspended in 200 mM TEAB/10% ACN, mixed with their respective TMT8plex reagent (15 mM final concentration), as shown in labelling design below, and left to incubate for 1 hour at room temperature. The TMT reactions were then terminated with 0.25% hydroxylamine for 15 minutes. Samples were pooled into three TMT8plex (labelling design shown below) and left to incubate for another 15 minutes. Each TMT8plex sample were acidified and the acentonitrile concentration diluted to below 5%, then divided into three aliquots each of which were desalted on a 200 mg SepPak tC18 cartridge, eluted, then lyophilized. Labeling design shown in Table 8.

### SCX-HPLC.

All 9 aliquots of lyophilized peptides (Table 9) were re-suspend in SCX buffer C, then separated into 12 fractions by SCX-HPLC. The fractionation was carried out using a polySULFOETHYL-A column (PolyLC) and our SCX HPLC system (Waters Alliance 2695) according to the Villén and Gygi, Nature Protocol26, approach.

Buffer A: 0,1% TFA in water.
Buffer C: 7 mM KH2PO4, pH 2.65, 30% ACN (vol/vol).
Buffer D: 7 mM KH2PO4, 350 mM KCl, pH 2.65, 30% ACN (vol/vol).

### Immobilized Metal-Affinity Chromatography (IMAC) and TiO₂.

Phosphopeptides were enriched by IMAC (Thermo Scientific Pierce product code 88300) or TiO2 (Thermo Scientific Pierce product code 88301), in accordance with manufacturer's instructions.

### Graphite Spin Columns.

Following phosphopeptide enrichment, peptides were purified using graphite spin columns (Thermo Scientific Pierce product code 88302), according to manufacturer's instructions.

### Liquid Chromatography Mass Spectrometry (LC-MS).

Peptides from all 108 fractions were re-suspended in 35 μl of 2% ACN, 0.1% FA, then 8 μL of each sample were injected onto a 0.1×20 mm pre-column self-packed with ReproSil C18, 5 μm (Dr. Maisch), using the Thermo Scientific Proxeon EASY-nLC II system. Peptides were then resolved using an increasing gradient of 0.1% formic acid in acetonitirile (10 to 25% over 90 minutes) through a 0.075×150 mm self-packed column with ReproSil C18, 3 μm (Dr. Maisch) at a flow rate of 300 nL/min. Mass spectra were acquired on a Thermo Scientific LTQ Orbitrap Velos throughout the chromatographic run (115 minutes), using 10 higher collision induced dissociation (HCD) FTMS scans at 15000 resolving power @ 400 m/z, following each FTMS scan (2×μScans at 30000 resolving power @ 400 m/z). HCD was carried out on 10 of the most intense ions from each FTMS scan then put on a dynamic exclusion list for 30secs (10 ppm m/z window). AGC ion injection target for each FTMS1 scan were 1000000 (500 ms max injection time). AGC ion injection target for each HCD FTMS2 scan were 50000 (500 ms max ion injection time). Each sample were analysed by three LC-MSMS analytical repeats, where the third analytical repeat used a time dependent rejection list, rejecting all peptide ions that were identified as peptides, with 1% FDR, in one of the first two analytical repeats.

### Peptide Identification and Quantification.

### Proteome Discoverer

In total there were 324 Raw data files (3×TMT8plex sample X3 aliquots X12 fractions X3 analytical repeats), where there were 108 raw data files belonging to each TMT8plex. All 108 raw data files from the first TMT8plex sample were combined for a Mudpit search using Proteome Discoverer, as described below. This was also carried out for the second and third TMT8plex samples.

Raw data were submitted to the Thermo Scientific Proteome Discoverer 1.3 software, using the Spectrum Files node. Spectrum selector was set to its default values, while the Mascot node was set up to search data against the uniprot sprot database, taxonomy homo sapiens. This node was programmed to search for tryptic peptides (two missed cleavages) with static modifications of carbamidomethyl (C), TMT6plex (K), and TMT6plex (N-Term). Dynamic modifications were set to deamidation (N/Q), oxidation (M), and phosphorylation of STY. Precursor mass tolerance was set to 20 ppm and fragment (b and y ions) mass tolerance to 20 mmu. Spectra were also searched against SEQUEST, using the same database, modifications, and tolerances as the Mascot node. Spectra were also search using the PhosphoRS2.0 (fragment mass tolerance of 20 mmu, considering neutral loss peaks for CID and HCD) and Percolator nodes.

The reporter ions quantifier node was set up to measure the raw intensity values of TMT8plex mono-isotopic ions, from all identified PSMs, at; 126.12773 m/z (126), 127.12476 m/z (127e), 127.13108 m/z (127), 128.13444 m/z (128), 129.13147 m/z (129e), 129.13779 m/z (129), 130.14115 m/z (130), 131.13818 m/z (131), using a tolerance of 20 ppm after centroiding. No filters were applied at this stage using Proteome Discoverer, therefore all raw intensity values were exported to excel for later processing and filtering using in house software.

### Bioinformatics

Statistical analysis was performed to investigate relevant regulations with respect to the disease group of T (pancreatic tumor tissue) and NT (non-tumor tissue) from 12 patients.

Accuracy and precision of mass spectrometry quantification approaches can suffer from issues such as Experimental bias, Systematic errors, Random Errors (Heterogeneity of Variance), and missing quantification values. To improve accuracy and precision the inventors assessed the quality of their data, then filtered and normalised as described below.

### MS Quality—Data Filtering and Normalisation:

All spectra which did not include all TMT-8 plex reporter intensities were deleted. For nomalisation a sum-scaling was performed. Due to differences between samples it is advisable to normalize data before further processing. The effects of the normalization can be observed by the follow maplots (http://en.wikipedia.org/wiki/MA_plot).

### Statistics

As first step log 2 ratios are calculated, which show the regulations T (pancreatic tumor tissue) over NT (non-tumor tissue) for all and for each patient. For protein ratios all peptides which are not phosphorylated were used and combined with the median.

The ratios were calculated:

log₂(ᵢT/ᵢNT)
Where i=patient 1,4,5,6,7,8,9,10,11,12,13,14

For the data analysis a one sided t-test (or one-sample location test) will be used [http://en.wikipedia.org/wiki/T_test]. A one side t-test is able to detect significant regulations in the subject of the question.

P-values and log 2 ratios can be observed in the attached list of interest (Table 5). Significant p-values were highlighted in red. Annotation with GO-terms, KEGG-pathways and Drugbank info were added at the protein level, and annotation from phosphosite plus were added at the phosphorylation site level.

For the phosphopeptide ratios all peptides which have a probability in the phospho-RS utility in the Proteome Discoverer from over 75% in any phosphorylation position was used.

### Results and Discussion

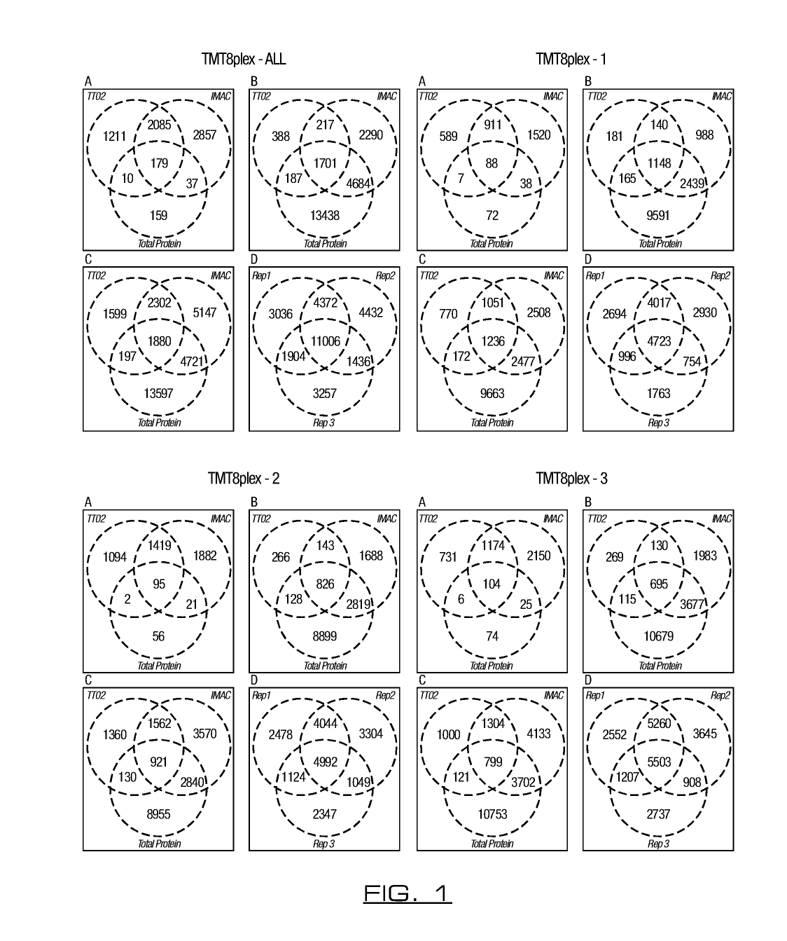

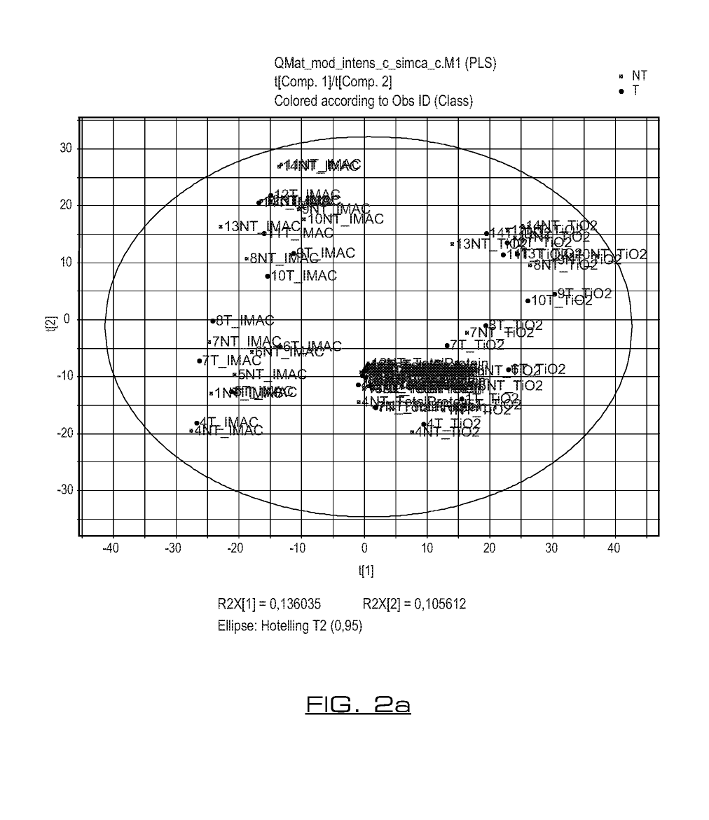

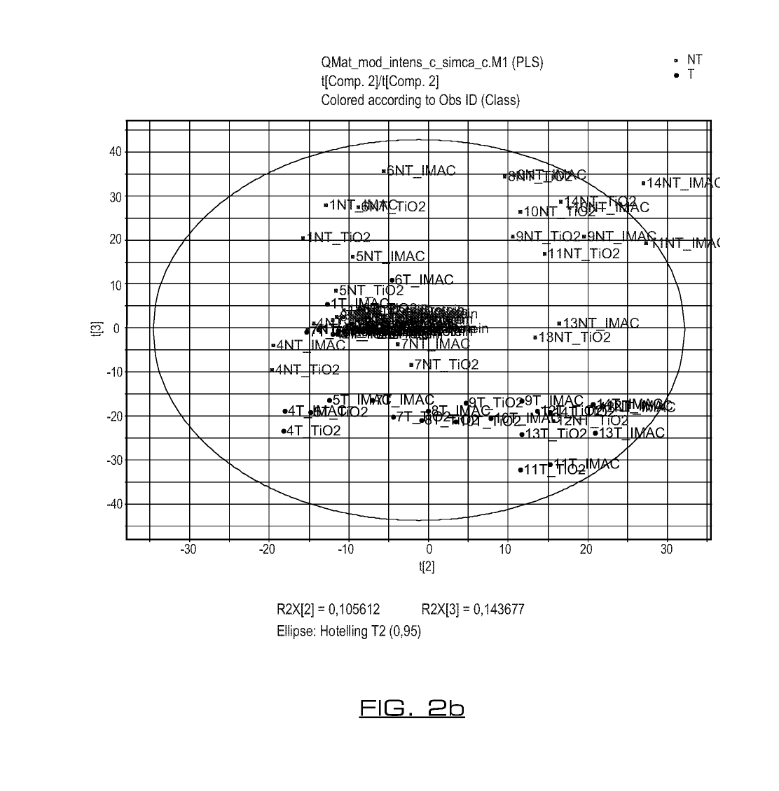

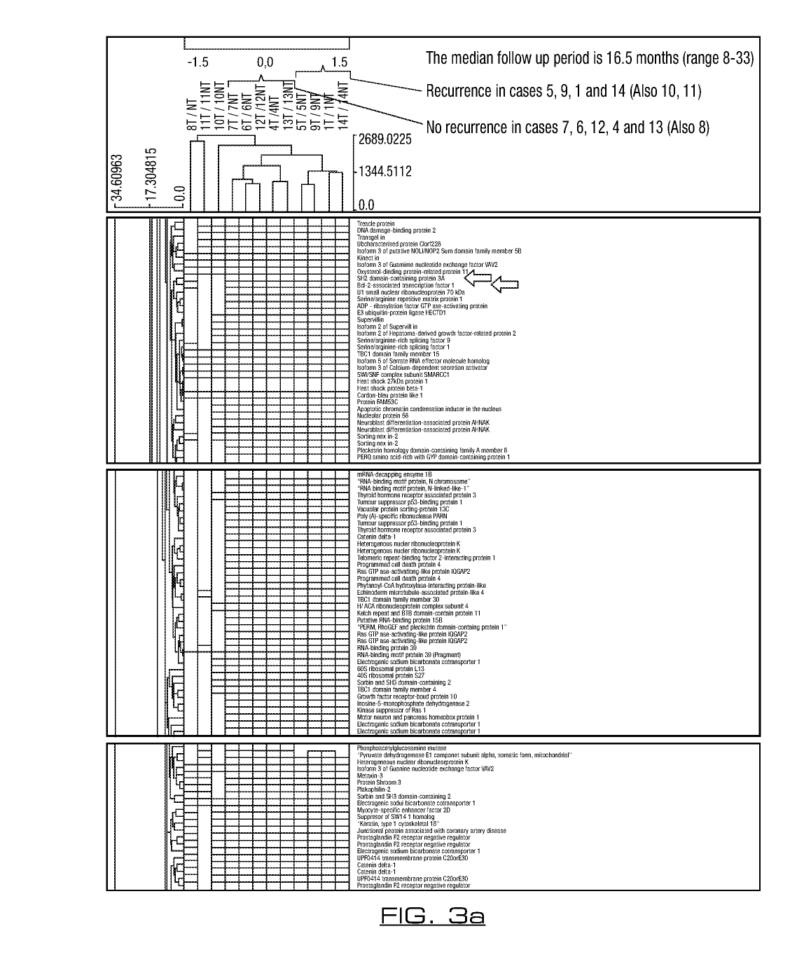

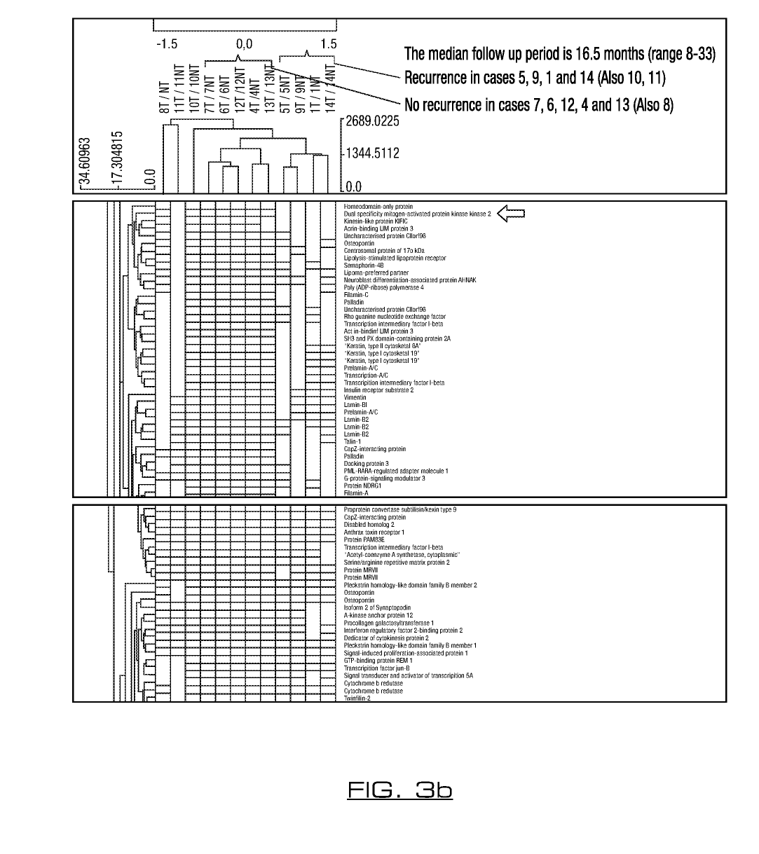

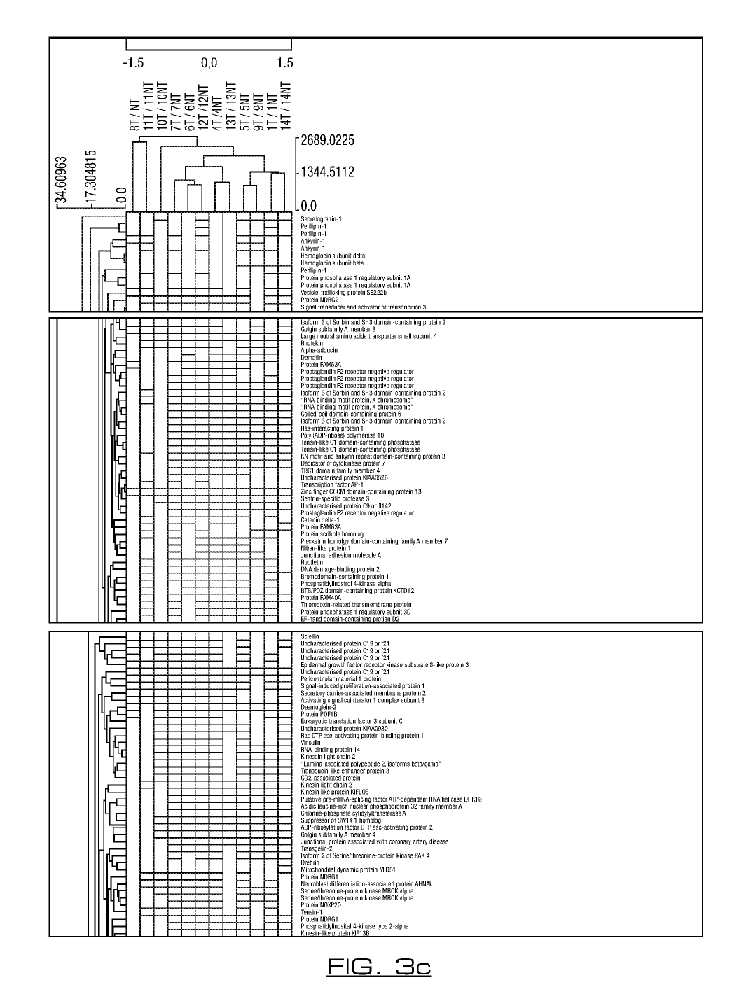

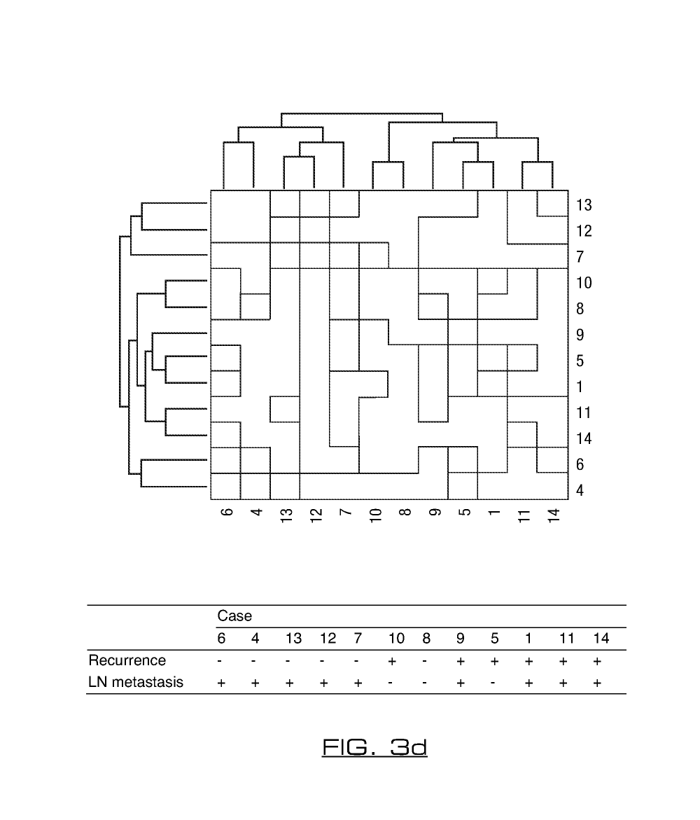

In total the inventors have identified 6,543 unique phosphopeptides (6,284 unique phosphorylation sites), from 2,101 protein groups (Table 1). FIG. 1 shows identified peptide (phosphorylated and non-phosphorylated) distribution over all the three arms (Non-enriched, TiO₂, IMAC) of the SysQuant workflow for each TMT 8-plex. FIG. 1 also illustrates the number of peptides detected for each of the three analytical repeats per sample. When results from each of the parallel components (TiO₂, IMAC, non-enriched) are compared the benefits of a combined approach are apparent. The largest total number of phospho-peptides was seen using IMAC enrichment which accounted for 79% of all unique phosphopeptides identified. However, the TiO₂ fractions uniquely identified nearly 19% of the total which would be missed using a single phospho-peptide enrichment strategy (FIG. 1:TMT8plex-ALL:a). The same is true for the three analytical runs performed on each sample. If a single data dependent run was performed only 20,318 unique peptides are seen (FIG. 1:TMT8plex-ALL:d). A second data-dependent run adds 5,868 peptides whilst the use of the time dependent rejection list in run 3 allowed a further 3257 peptides to be identified overall. Collectively (run 2&3) this represents an additional 45% over run 1 alone and 31% of the total number of unique peptides. Importantly the peptides identified in the third run are generally of lower abundance.

### PLS/PCA

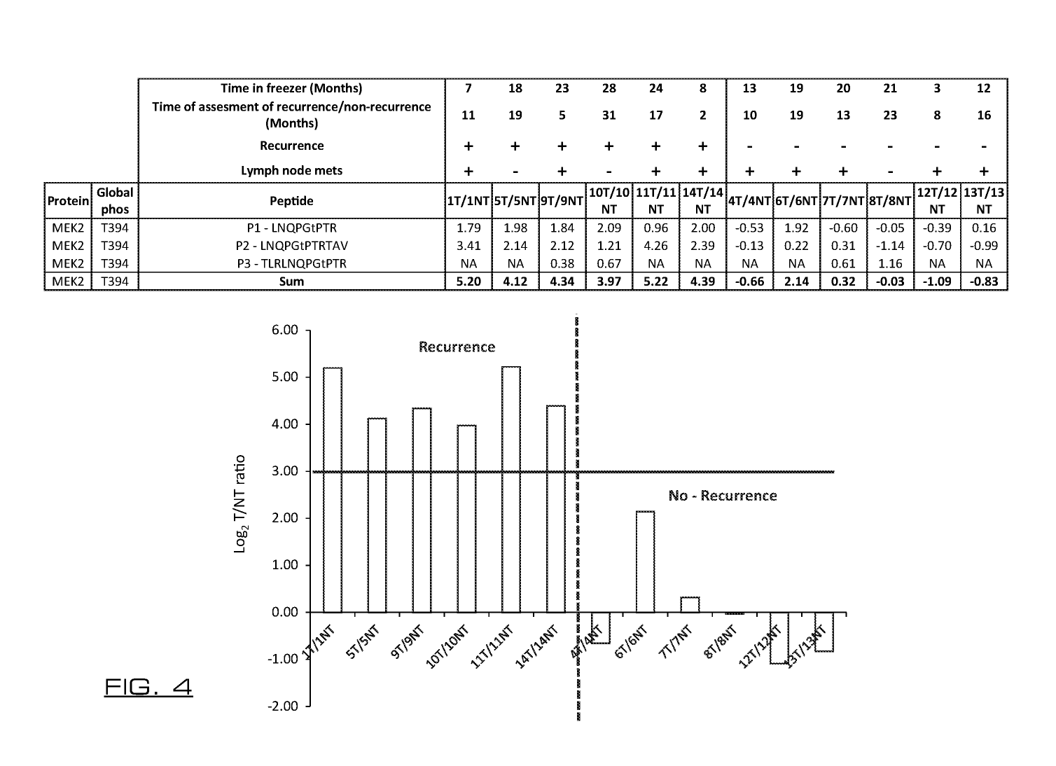

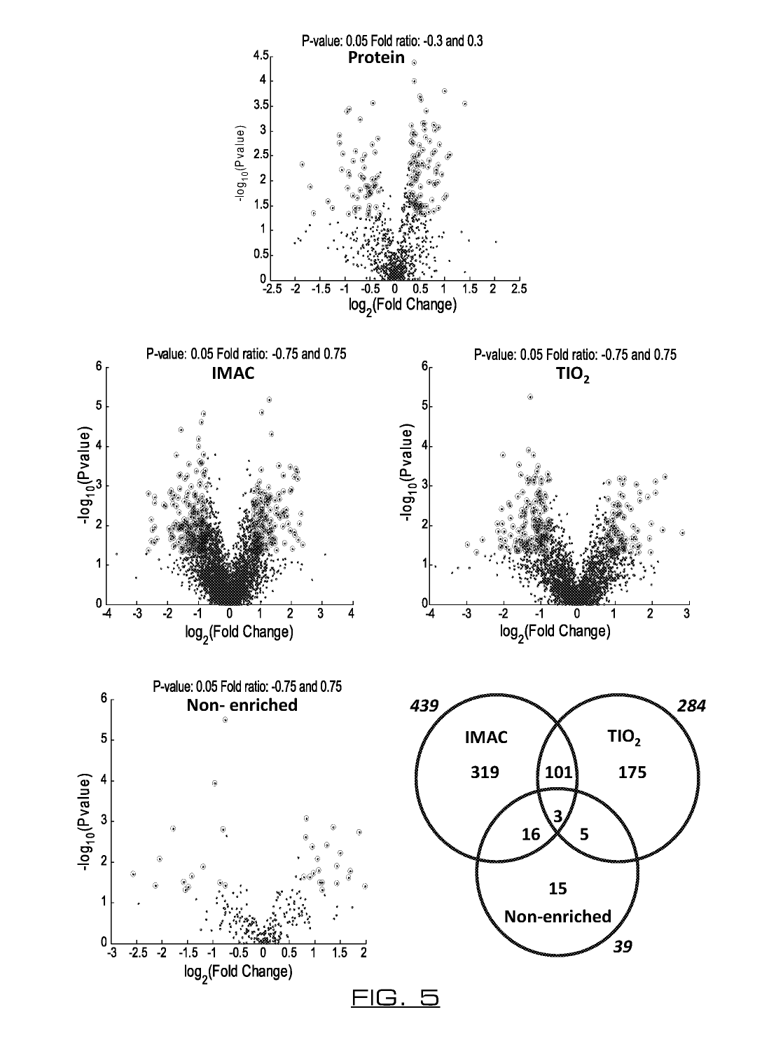

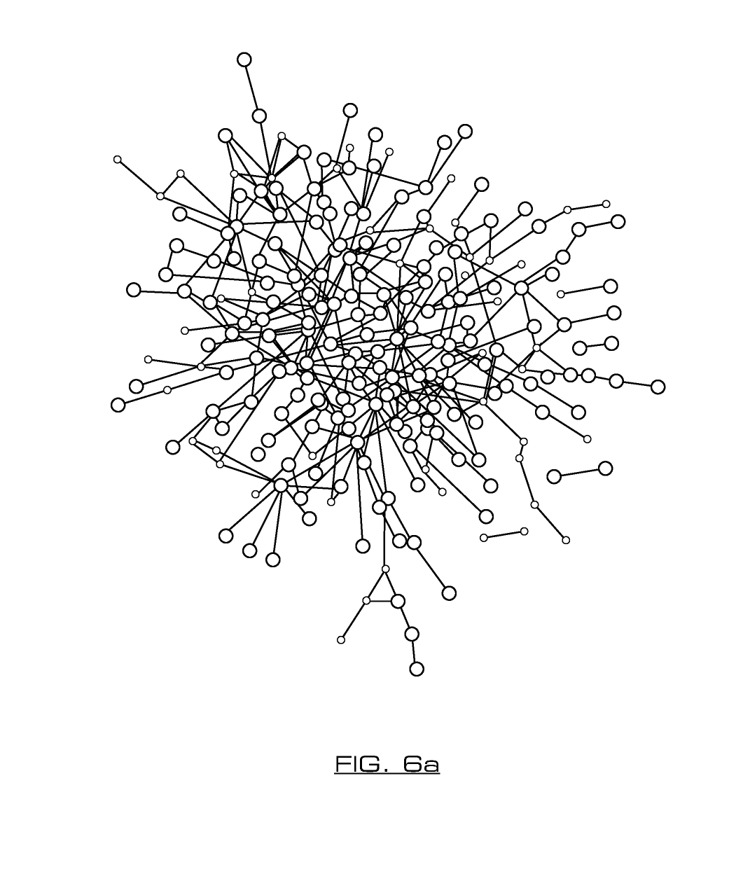

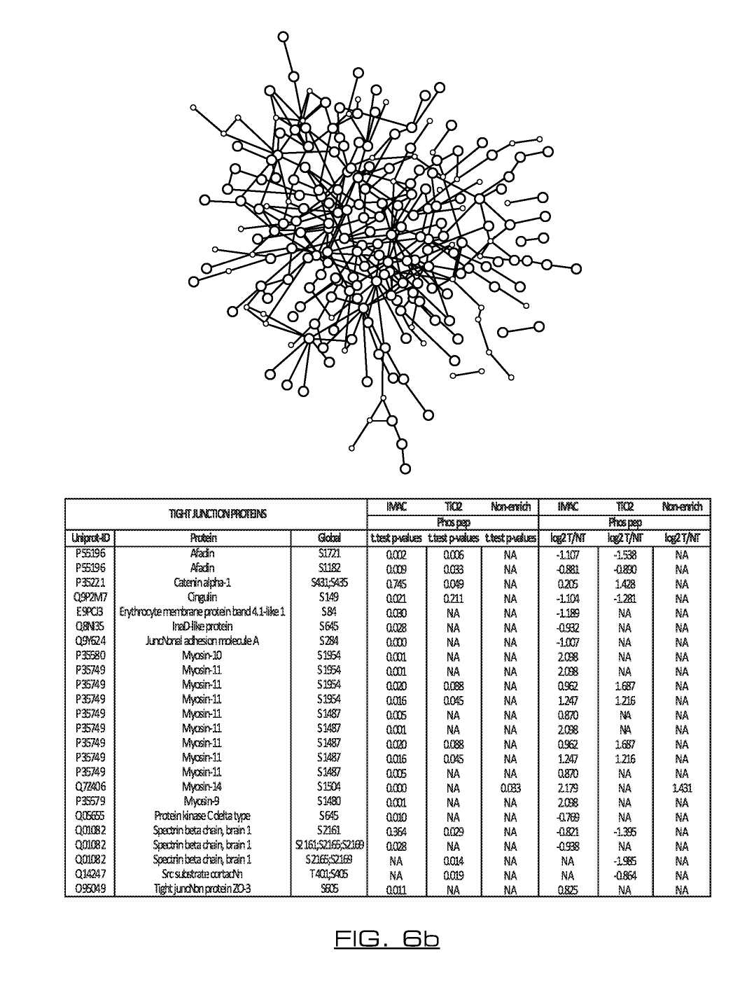

PLS demonstrated that there are no outliers in this dataset. PLS PC1 and PC2 show that there are three clusters IMAC, TiO₂ and TotalProtein (i.e. non-enriched arm of workflow), as shown in FIG. 2A. PC1 and PC2 Score plot of the first two principal components describing 13.6% (PC1) and 10.6% (PC2) of the total variance in the data. The circle depicts the T2 hotelling space based on 95% confidence. All samples were in the border of the model. PC1 refers to the enrichment, PC2 refers to the patient. TotalProtein (non-enriched peptides) has a cluster which is different to the enrichment arms of the workflow, IMAC and TiO₂ (FIG. 2A). PC2 and PC3 Score plot of the next principal components describing 10.6% (PC2) and 14.4% (PC3) of the total variance in the data (FIG. 2B). In PC3 PLS can split T and NT in two clusters. TotalProtein (non-enriched peptides) has its own cluster, but it can also be separated into the classes T and NT. Only in patient 12 were no differences in T compared to NT observed. PLS/PCA confirm that the experiment is successful, and that there are significant differences between T and NT. Differences between TiO₂, IMAC and Totalprotein (non-enriched) exists, but TiO₂ and IMAC have a nearly equal correlation.

### Hierarchal Cluster Analysis

Hierarchal cluster analysis was used to cluster cases which demonstrate similar profile in the relative abundance of these 5409 phosphopeptides in T relative to NT (FIG. 3A-3C show particular regions of interest). Using all 5409 unique phospho-peptides the 12 patients could be clustered into three independent groups. One cluster contained cases 5, 9, 1, and 14, a second cluster contained cases 7, 6, 12, 4 and 13, while cases 8, 10, and 11 separated to a third cluster and were less closely related to each other than members of the other two clusters. Interestingly, when the clinical history of the 12 patients was un-blinded, the inventors found that cases 5, 9, 1, and 14 were patients that suffered tumor recurrence between 8 & 33 months (median follow-up period 16.5 months) after removal of the tumors analysed in this study, whereas cases 7, 6, 12, 4 and 13, were patients with no recurrence in this same time period. For more details on patient history refer to Tables 6 and 7. Of the three outliers two were from patients with subsequent recurrence (Cases 10 and 11) and one was from a non-recurrent patient (Case 8). It is interesting that 2 out of the 3 outliers had less advanced stage IIA (pT3N0M0) compared to the recurrent (4/4 stage IIB, pT3N1M0) and non-recurrent (4/5 stage IIB, pT3N1M0) clusters. Further refining of the cluster analysis was performed by clustering on Pearson's correlation coefficients. The Pearson's correlation coefficients were obtained by comparing all phospho-peptide log 2 T/NT values across all cases (FIG. 3D). This refinement of cluster analysis better separates the recurrent and non-recurrent cases.

Hierarchal cluster analysis clearly separated patients into groups dependent on recurrence and no recurrence therefore the inventors were particularly interested in identifying those phosphopeptides whose abundance correlated positively and inversely with recurrence as these may prove useful prognostic markers and help forecast the likelihood of recurrence in new patients after analysis of their resected T & NT tissue. These phosphopeptides can be viewed in Table 11. Table 11 displays all phosphopeptides displaying high (log 2 T/NT ≧0.7) and low (log 2 T/NT ≦−0.7) levels in tumor versus non-tumor from cases with recurrence that clustered together in FIG. 3D. The combined list of phosphopeptides in Table 11 provides useful prognostic markers helping clinicians predict patients who will go on to present recurrence before 31 months after surgery.

In addition to the differences in global profiles between T and NT there are many individual phosphorylation site changes of particular interest. As an example, the relative abundance profile of the phosphopeptides containing phospho-T394 of Dual specificity mitogen-activated protein kinase kinase 2, as seen on FIG. 2B (highlighted with a red arrow), and on FIG. 4 correlate positively with patients who suffered tumor recurrence at median 16.5 months. They were substantially increased in T relative to NT in all cases showing recurrence, and down or only slightly increased in T relative to NT in all cases that did not show recurrence (FIG. 4). This kinase is part of the RAS/RAF/MEK/ERK signaling pathway known to be down stream of RAS and RAF, but upstream of ERK1/2. K-RAS gene is mutated to an oncogenic form in most pancreatic tumors, most commonly in the form of K-RAS^{G12D} [12]. Unfortunately no K-RAS peptides were detected in this study. However, measurement of phospho-T394 on Dual specificity mitogen-activated protein kinase kinase 2, which is downstream of K-RAS, may prove to be an important prognostic marker assisting prediction of time of recurrence. The UniProtKB/Swiss-Prot database the inventors used to search peptides does not contain K-RAS point mutations, explaining the lack of detected K-RAS peptides in this study. This emphasises the need for a database containing known oncogenic point mutations. Other RAS signaling proteins were identified to show significantly modulated phosphopeptides as seen in the STRING map (see below).

The inventors also performed hierarchal cluster analysis to cluster cases which demonstrate similar profile in the relative abundance of protein in T relative to NT, however the correlation between clusters and recurrence/non-recurrence was less obvious, suggesting that total levels of protein expression change less dramatically than phosphorylation and signifying the importance of our phosphopeptide analysis as a prognostic tool.

### Significantly Regulated Protein Expression

The inventors determined the relative abundance of proteins in tumor compared to non-tumor tissue, using median log₂ T/NT ratios of the non-phosphorylated peptides unique to each protein as surrogates to calculate the relative abundance of the respective proteins. A one sided t-test was used to calculate p-values and these were plotted against log₂ T/NT ratios on a volcano plot to detect significant (Log₂ T/NT ≧0.3 or ≦−0.3 and p≧0.05) regulations over all cases (FIG. 5A). In total there were 150 proteins significantly regulated based on Log₂ T/NT ≧0.3 or ≦−0.3 and p≦0.05 (Table 12). Table 2 displays the 12 most significantly upregulated proteins in tumor compared to non-tumor tissue, and also provides a description of any known function of each protein or association with cancer [13-31]. Overexpression of Mucin-1 is often associated with cancer and the inventor also found Mucin-1 to be significantly up-regulated in pancreatic tumor tissue. Interestingly the inventors found more significant up-regulated proteins than Mucin-1, some of which may prove to be more specific markers of pancreatic cancer, perhaps even new therapeutic targets e.g. Homeodomain-interacting protein kinase 1.

The inventors selected all accession numbers of significantly modulated proteins and uploaded these to the DAVID Bio-informatic resource to identify those KEGG signalling pathways most significantly modulated. The Focal Adhesion KEGG signaling pathway was most significantly modulated giving a Benjamini score of 1.0E-3. Significantly modulated Focal Adhesion proteins included; Talin-1, Filamin-A, Filamin-C, Vinculin, Filamin B, Fibronectin, Focal adhesion kinase 1, Zyxin, Talin-2, Protein phosphatase 1 regulatory subunit 12A, and Myosin light chain kinase, smooth muscle (Table 12). In fixed or immobile cells, focal adhesions are quite stable under normal conditions, while less so in motile cells, where focal adhesions are constantly assembled and disassembled as the cell establishes new contacts at its leading edge, breaking old contacts at its trailing edge.

Hepatoma derived growth factor was also upregulated in most tumor specimens and this was significant based on p-value (p≦0.05).

Of particular interest to the inventors was the determination that Myosin light chain kinase (MLCK) is significantly overexpressed in tumor compared to non-tumor tissue (median log 2 T/NT=0.5 & p-value=2.95E−02). MLCK is a Ca2+/calmodulin-dependent protein kinase that regulates a variety of cellular functions, such as, muscle contraction and cell migration, via phosphorylation of myosin light chain proteins. Since tumor cell migration is a key step in tumor spread, myosin light chain kinase (MLCK) may be regarded as a therapeutic target for preventing tumor spread. In fact, MLCK activation and expression have been found to be positively related with metastatic propensity.

### Significantly Regulated Phosphopeptides

Log₂ T/NT ratios of the phosphorylated peptides were used to calculate the relative level of phosphorylation at specific unique phosphorylation sites. The inventors used t-tests to calculate p-values and these were plotted against log₂ T/NT ratios on volcano plots for IMAC, TiO₂, and Non-enriched arms of the workflow, to detect significant (Log₂ T/NT ≧0.75 or ≦−0.75 and p≦0.05) regulations over all cases, as shown in FIG. 5B-5D. Of the 6,543 phosphopeptides identified in this study, 5409 were quantifiable (Data not shown). Of the quantifiable peptides, 635 showed significant regulation (FIG. 5B-5D).

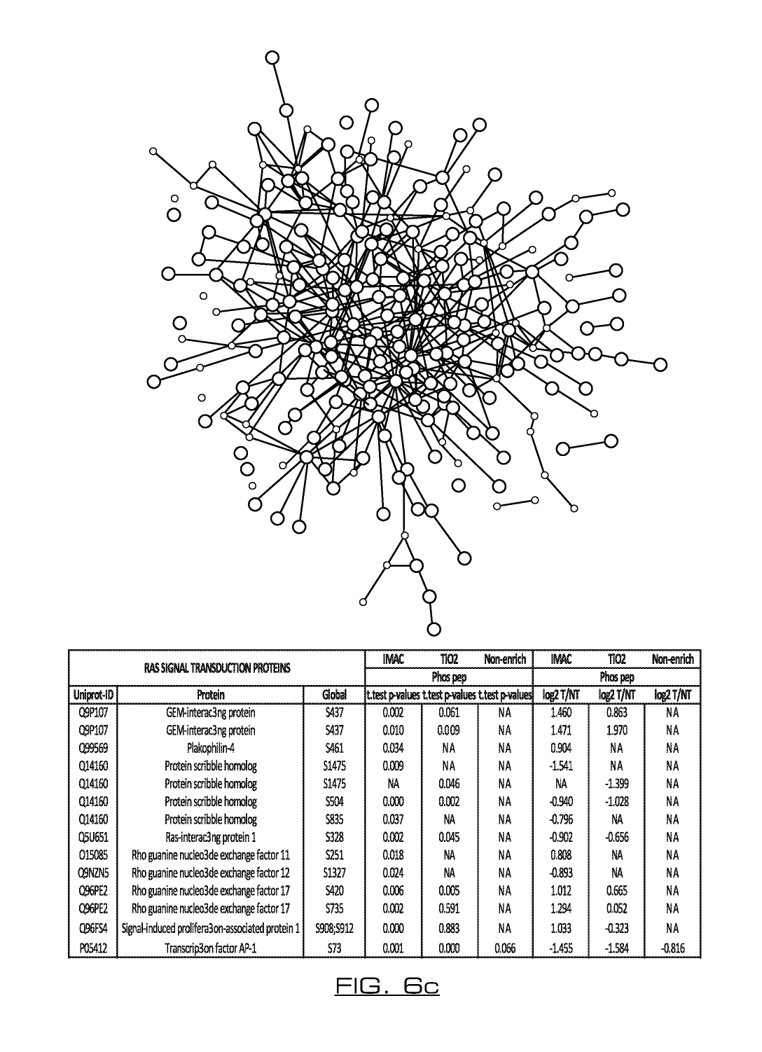

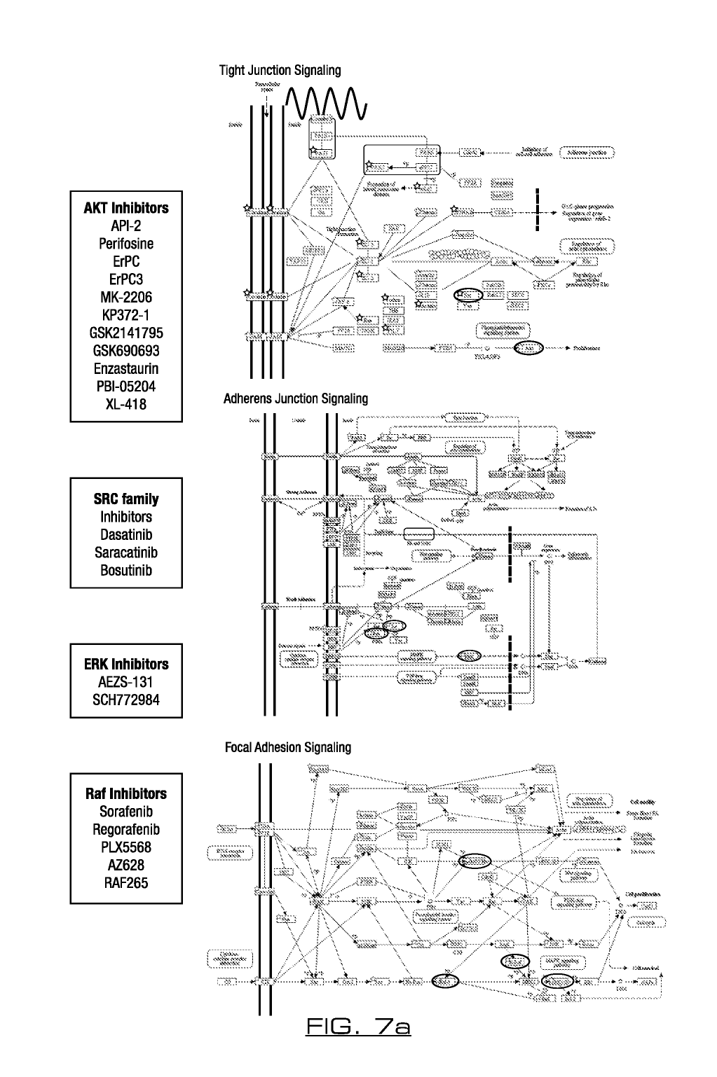

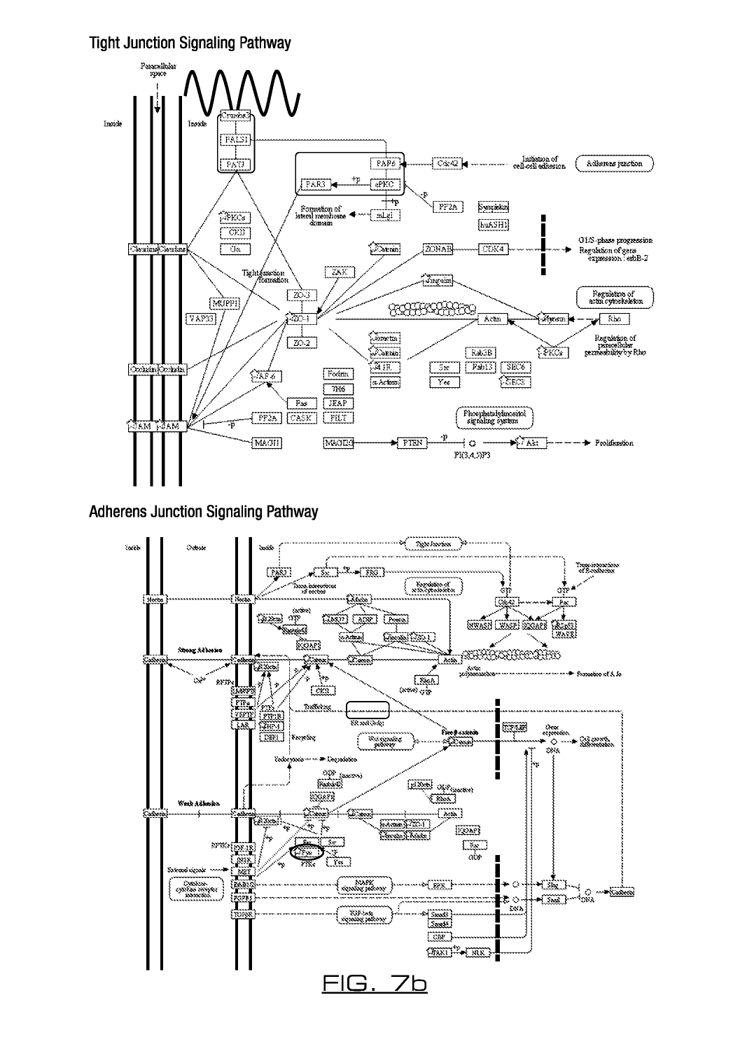

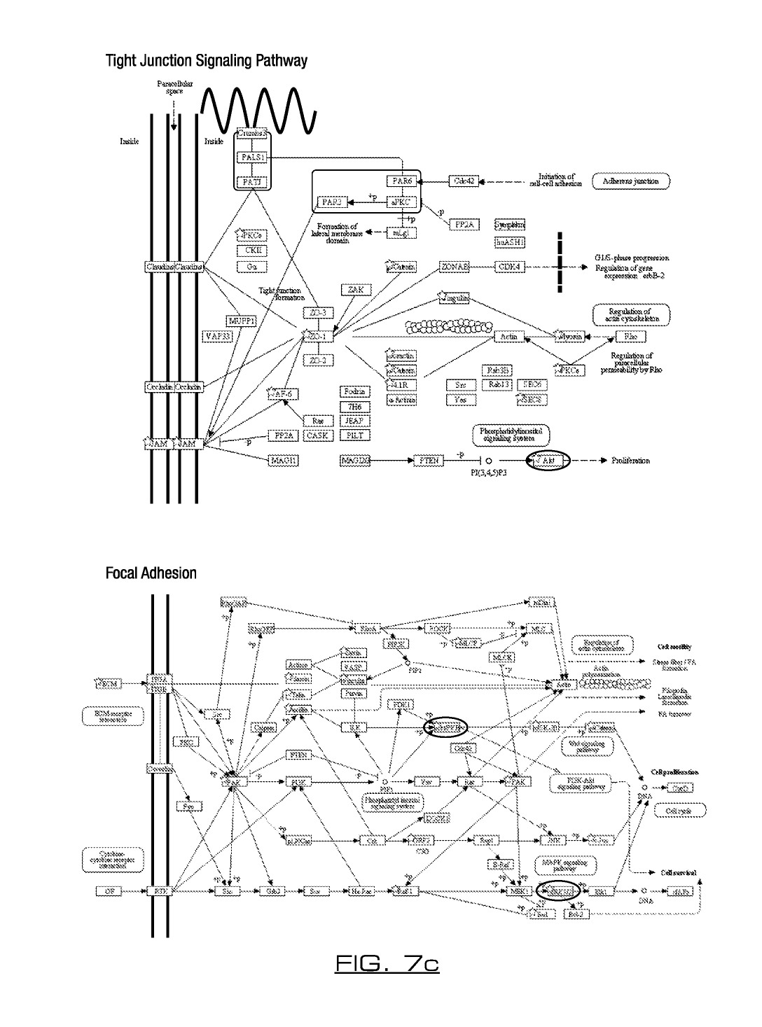

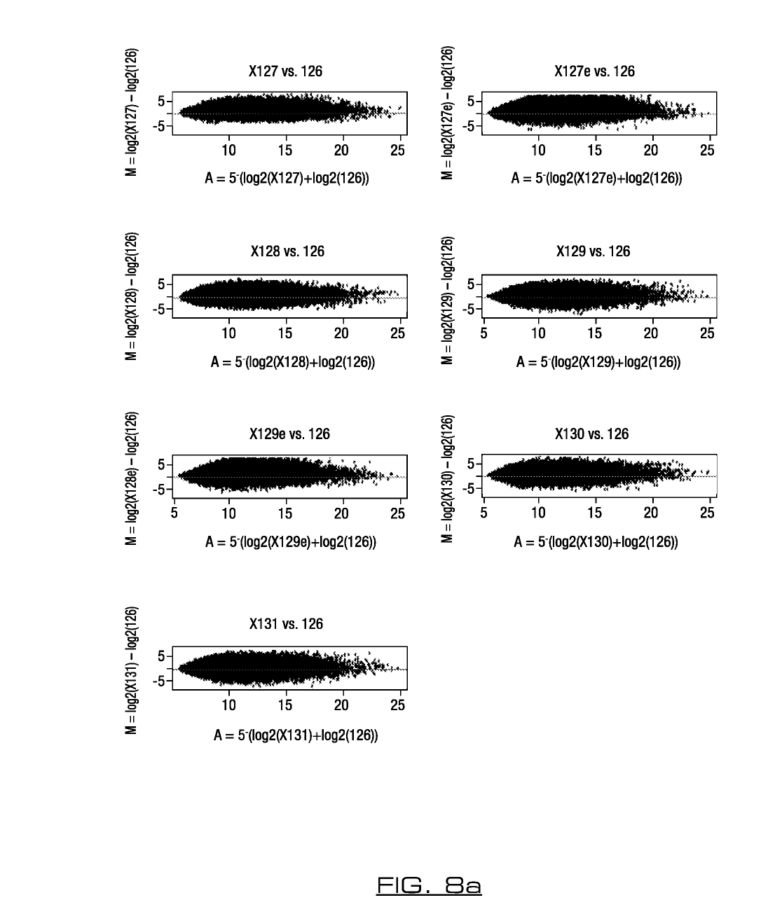

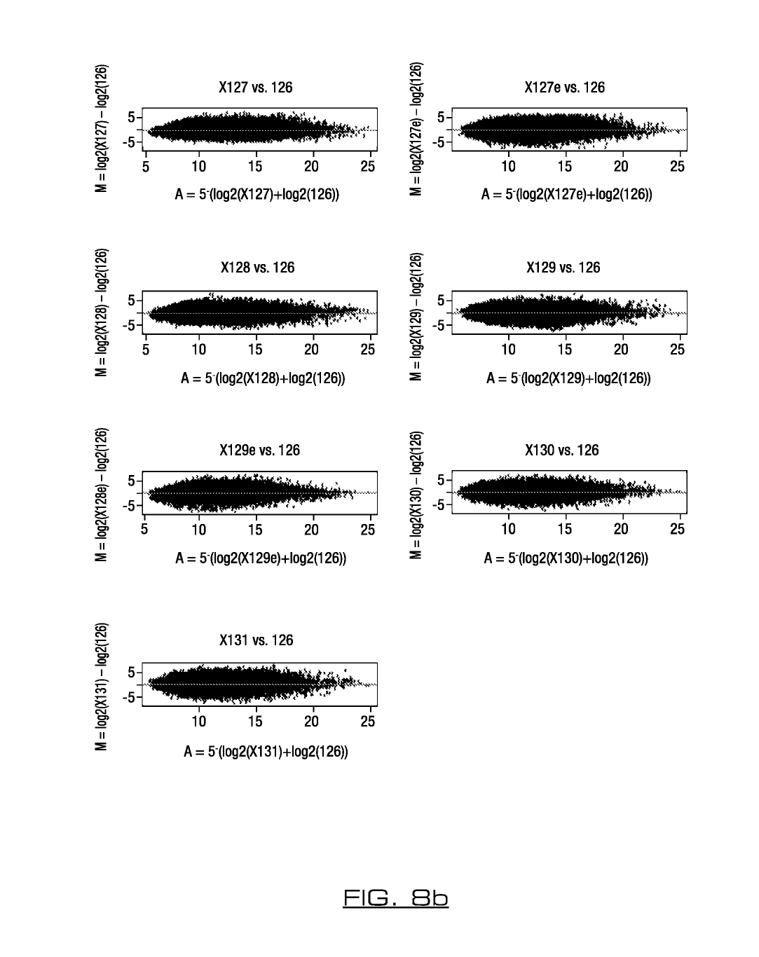

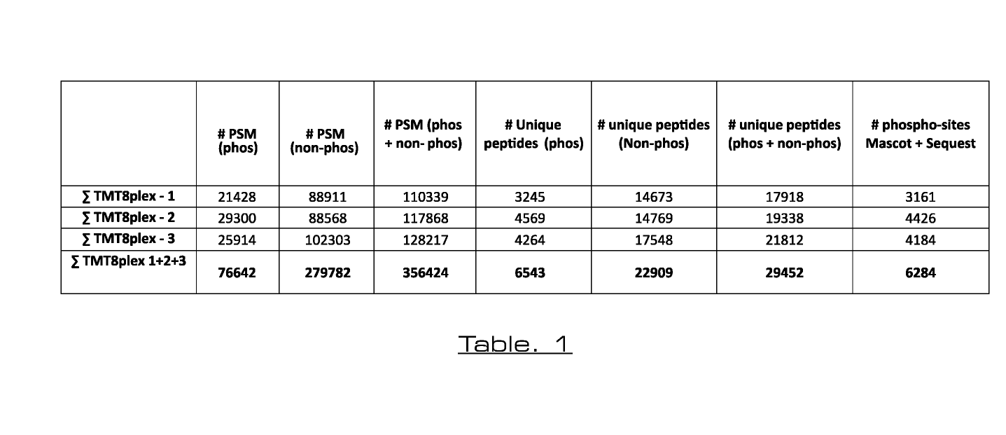

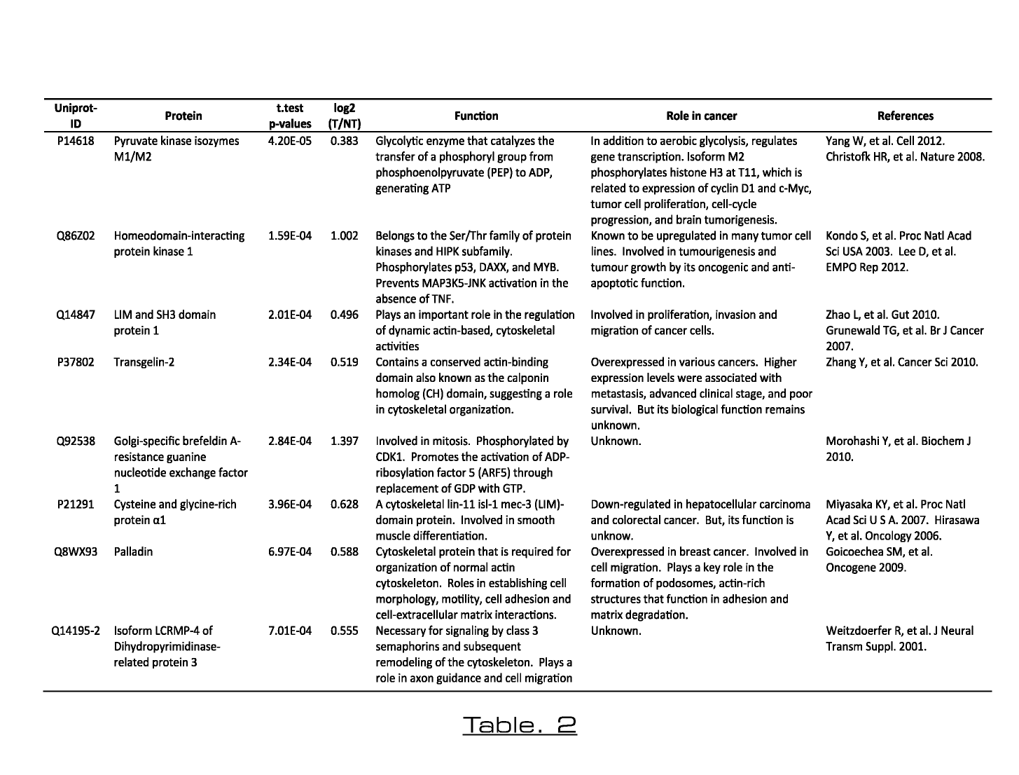

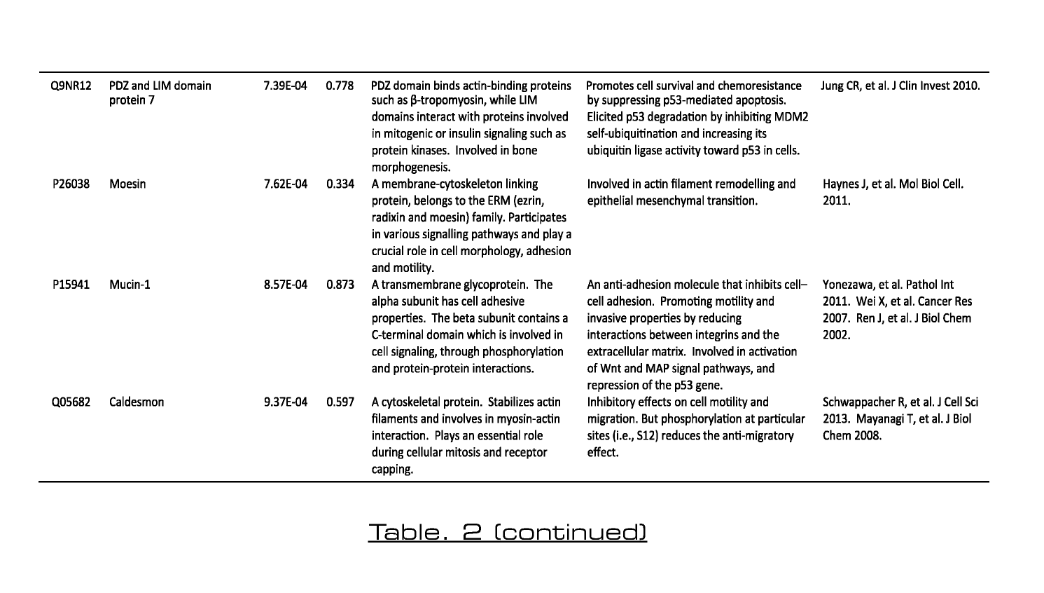

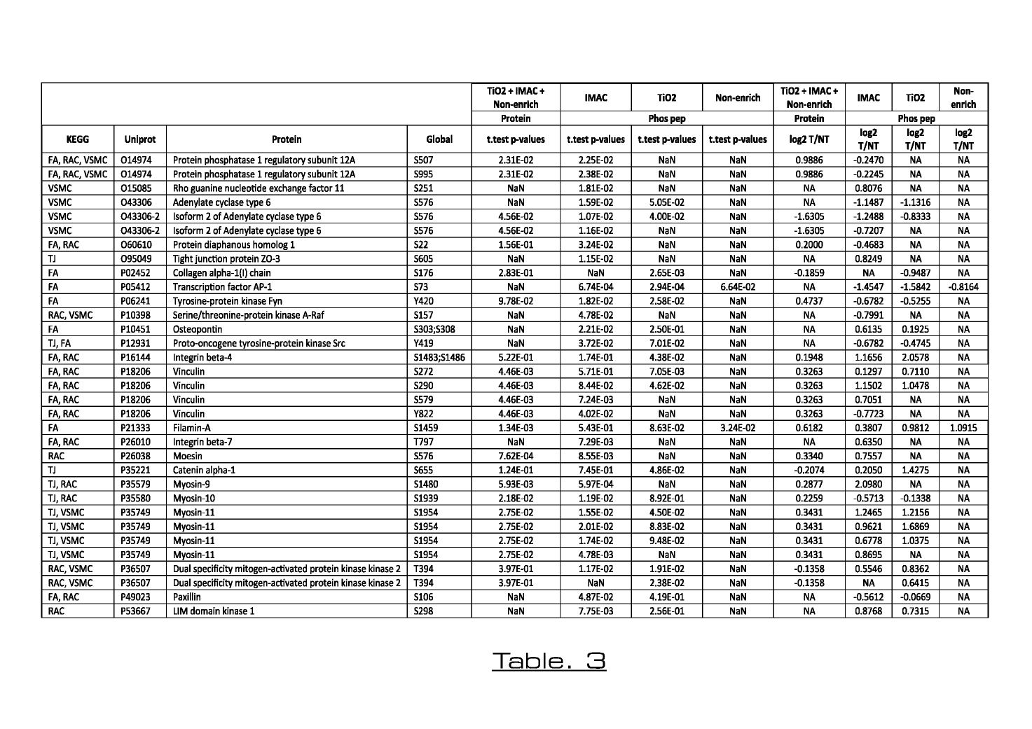

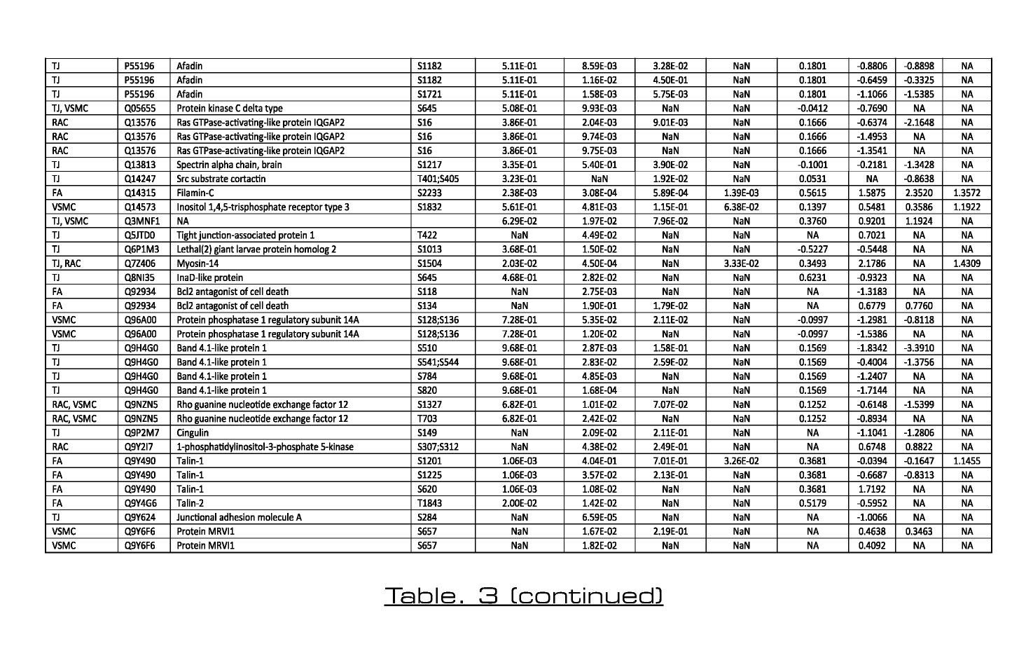

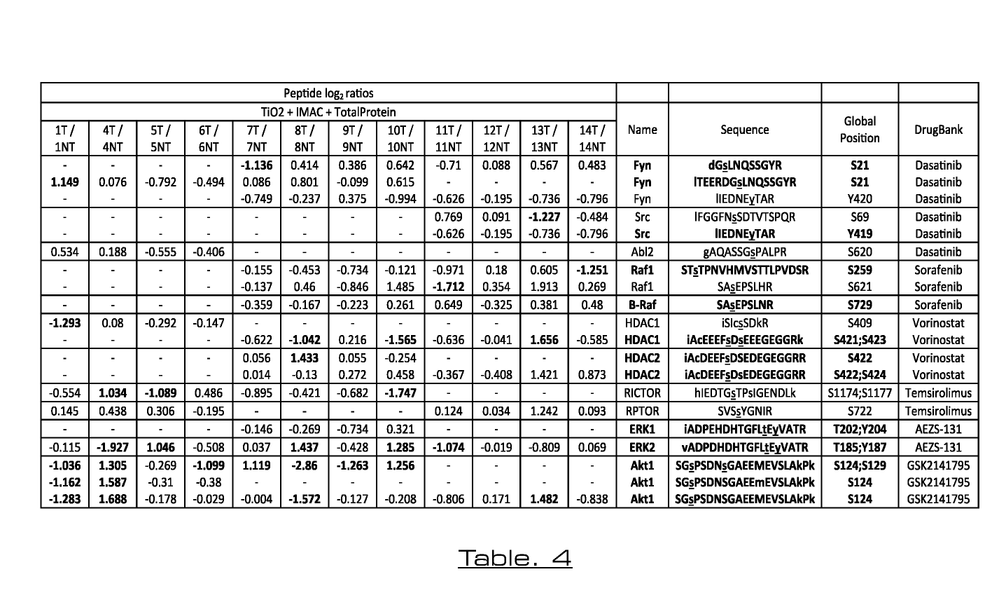

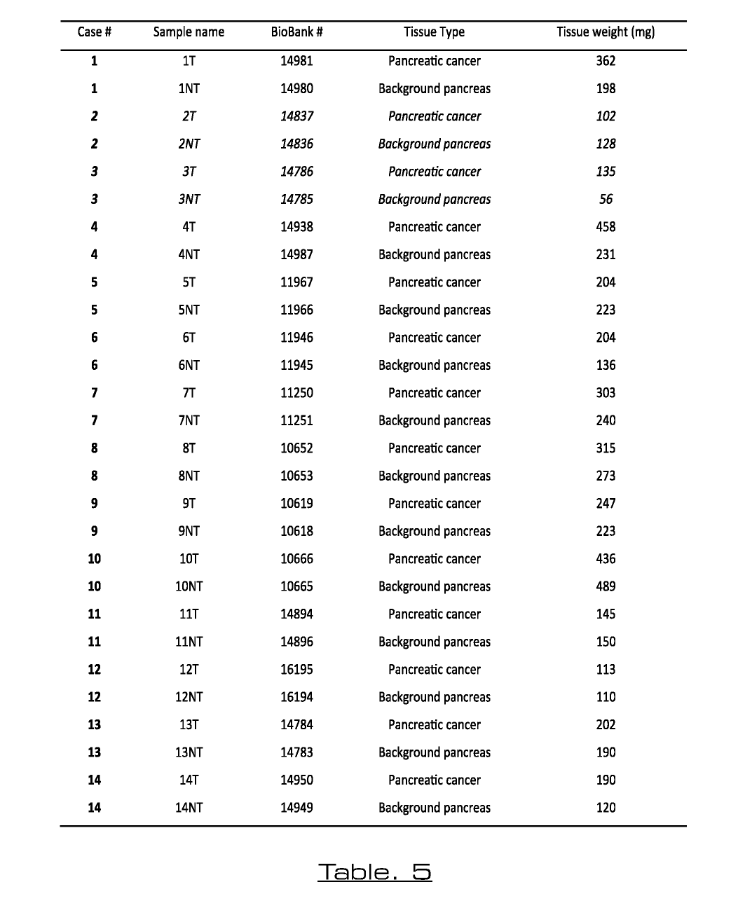

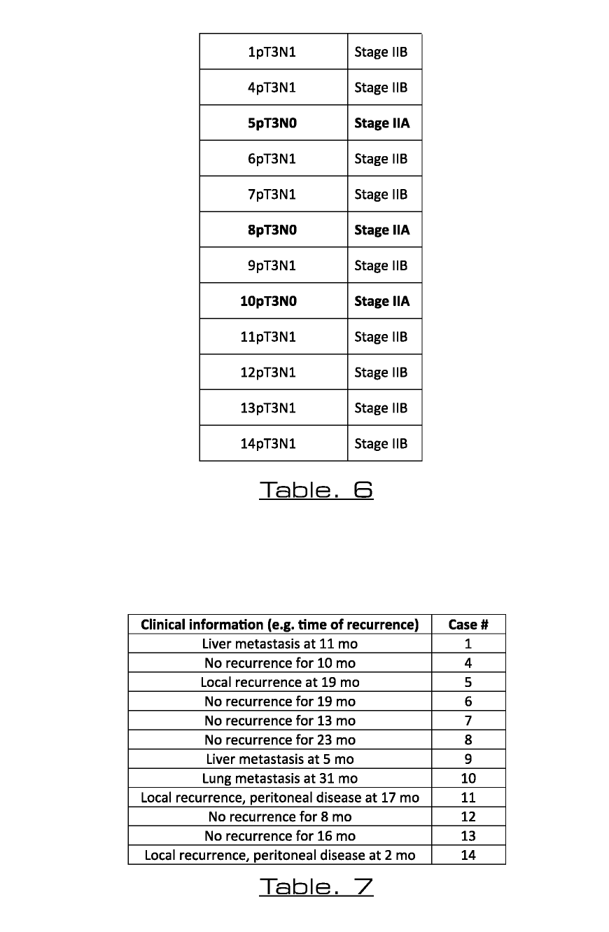

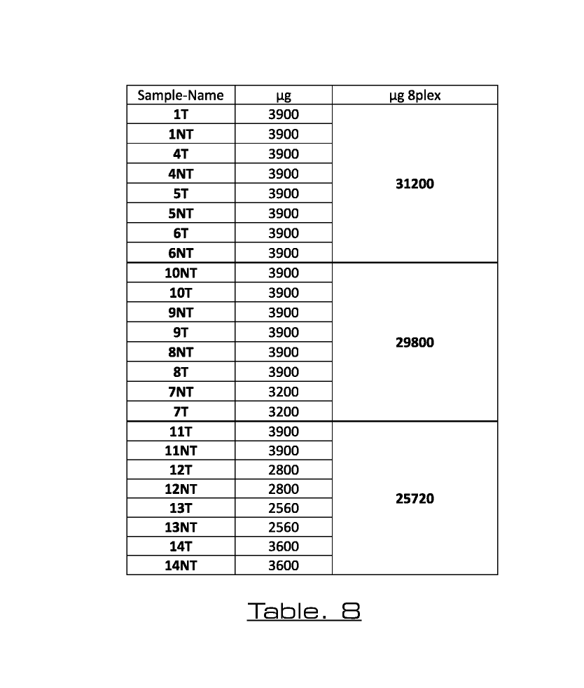

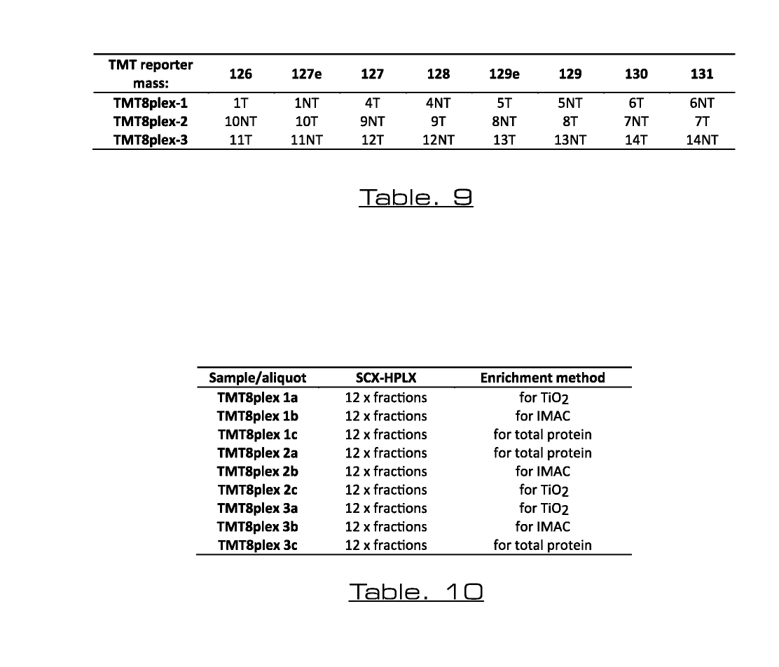

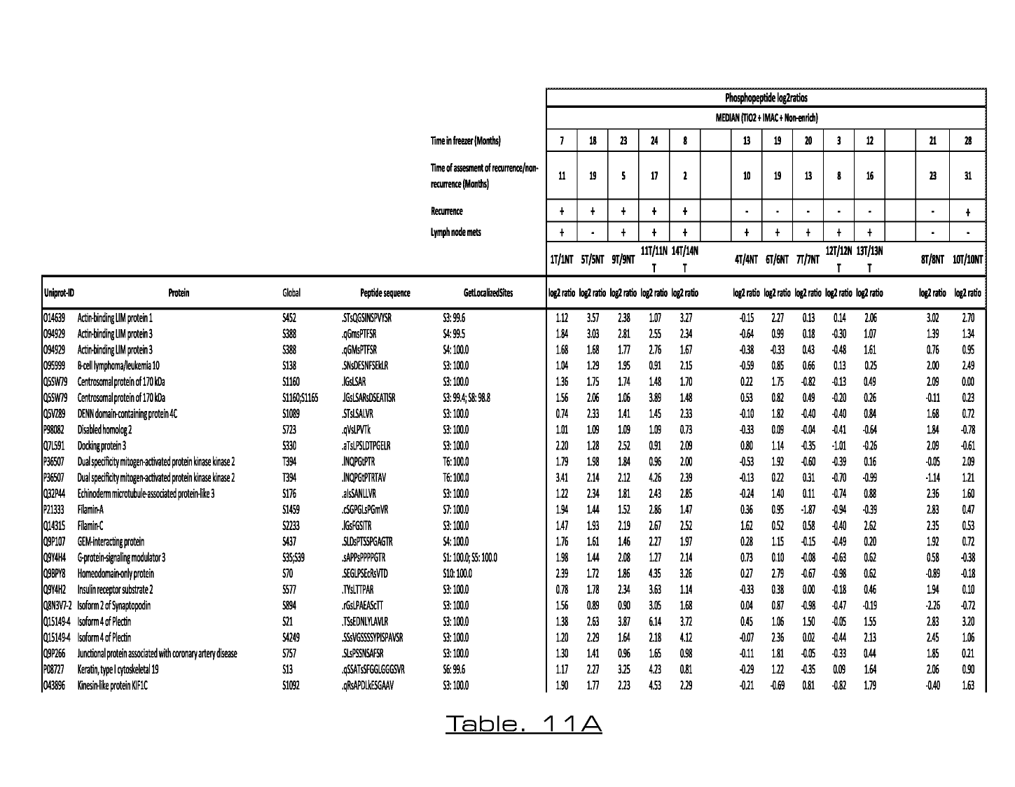

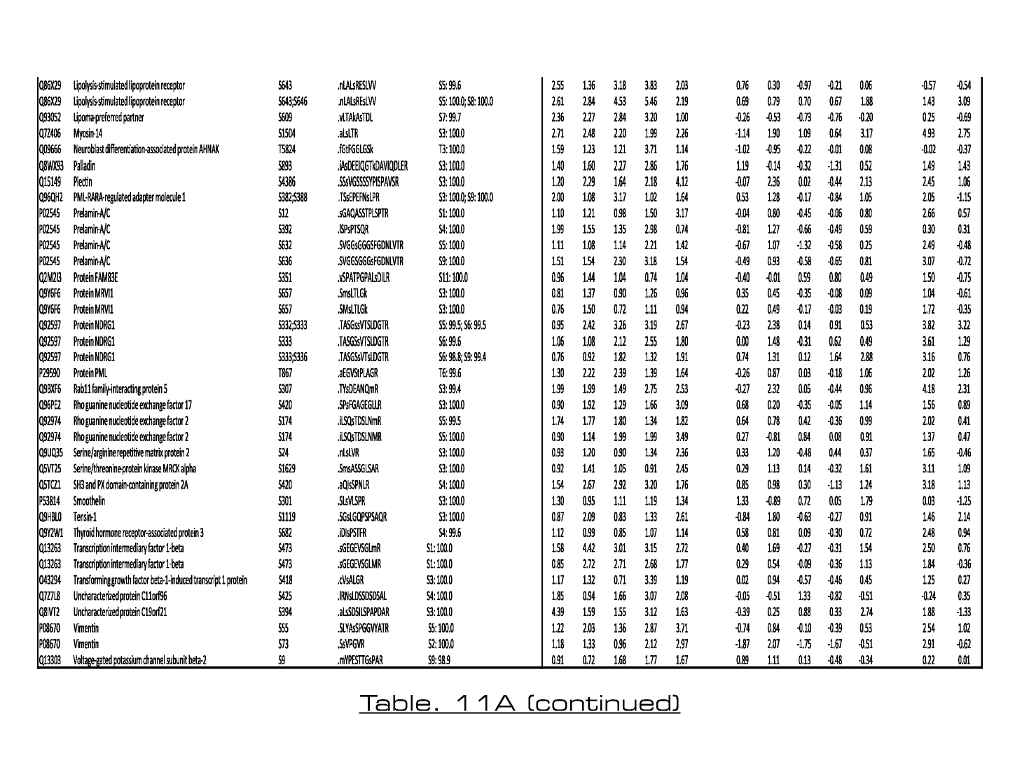

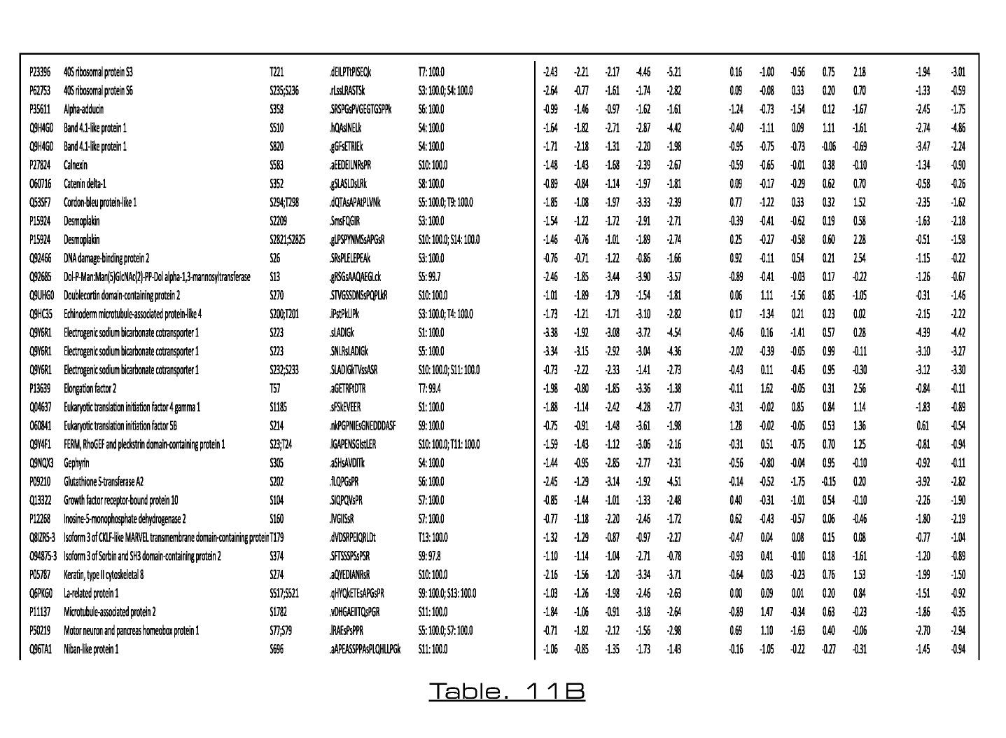

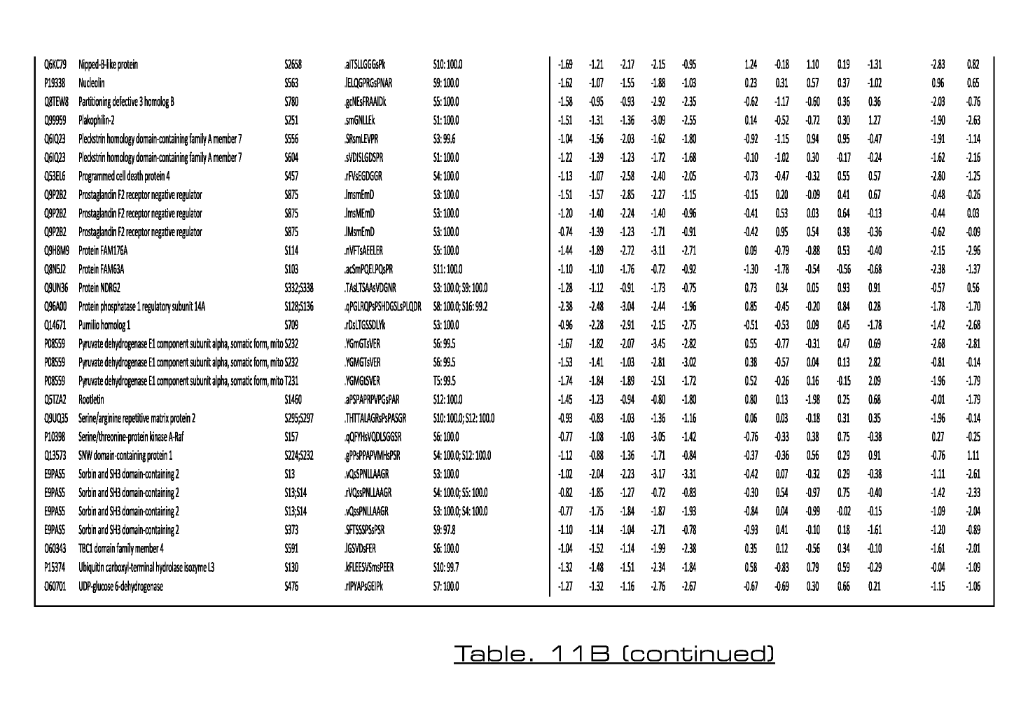

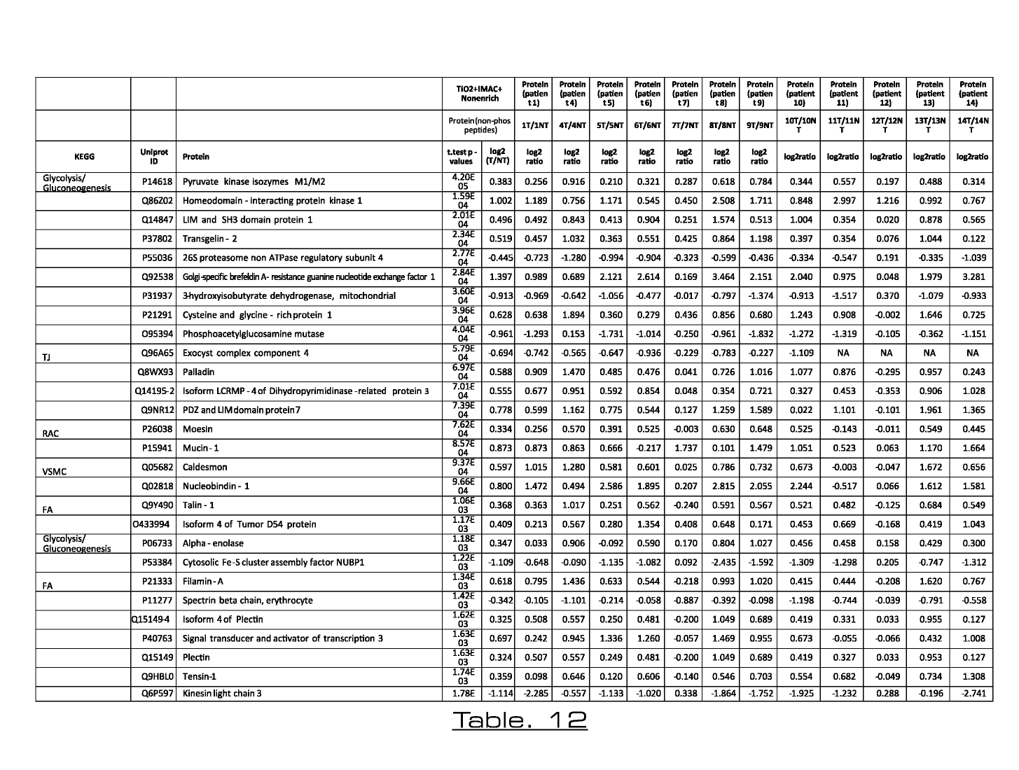

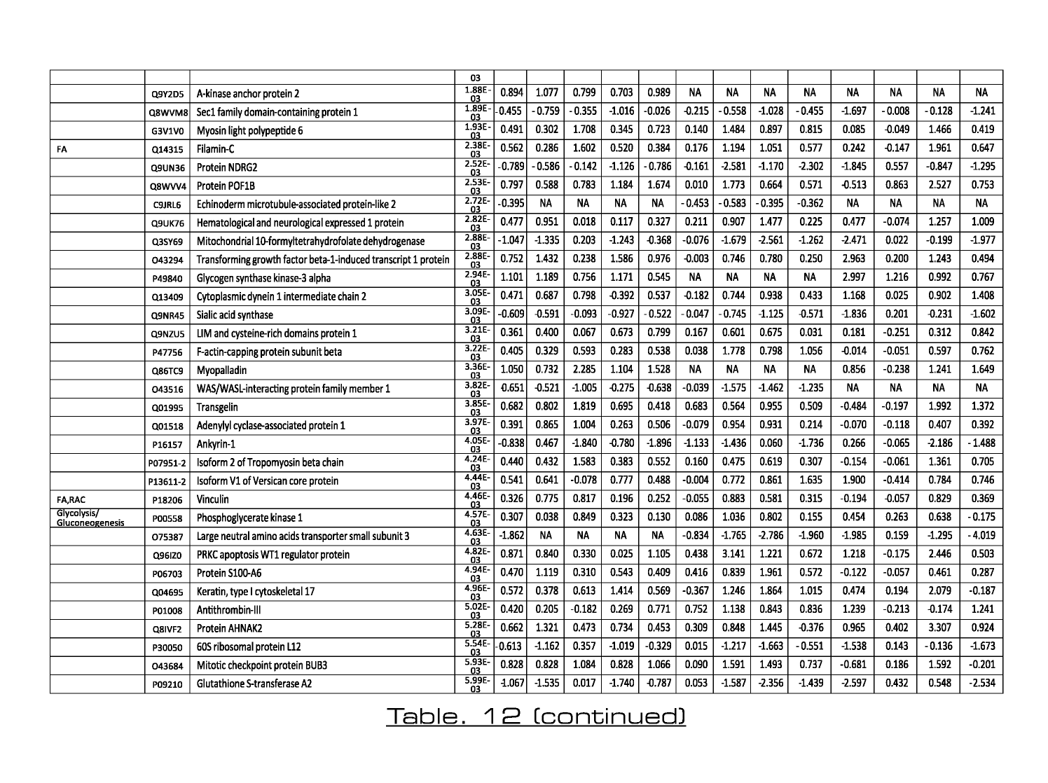

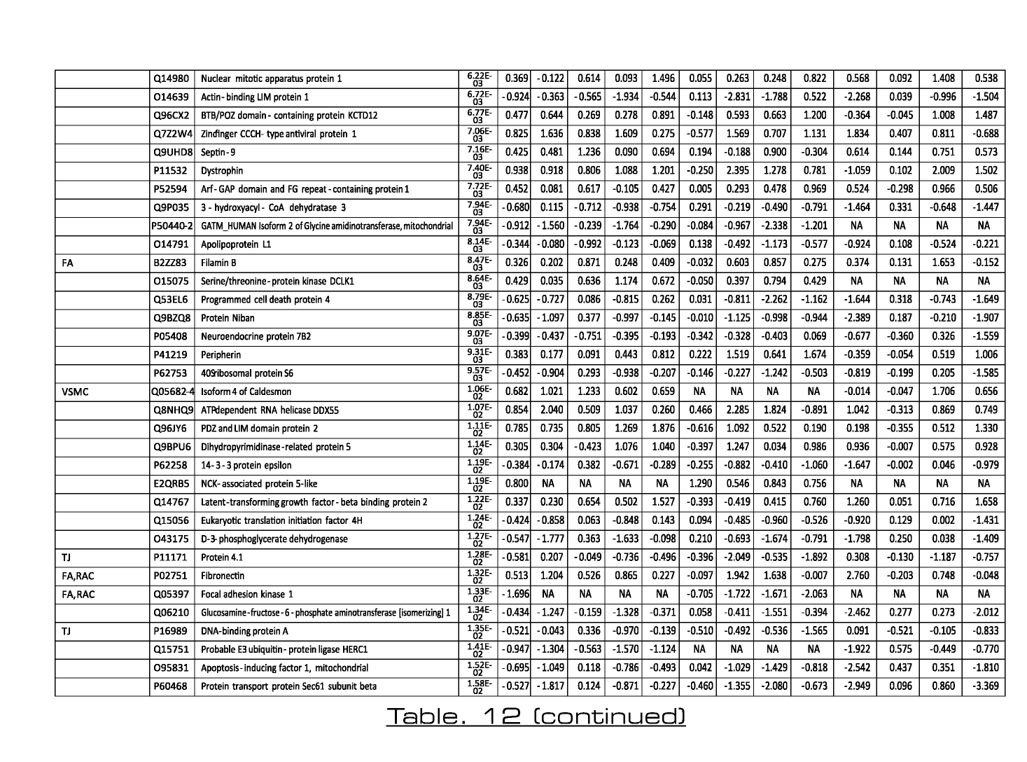

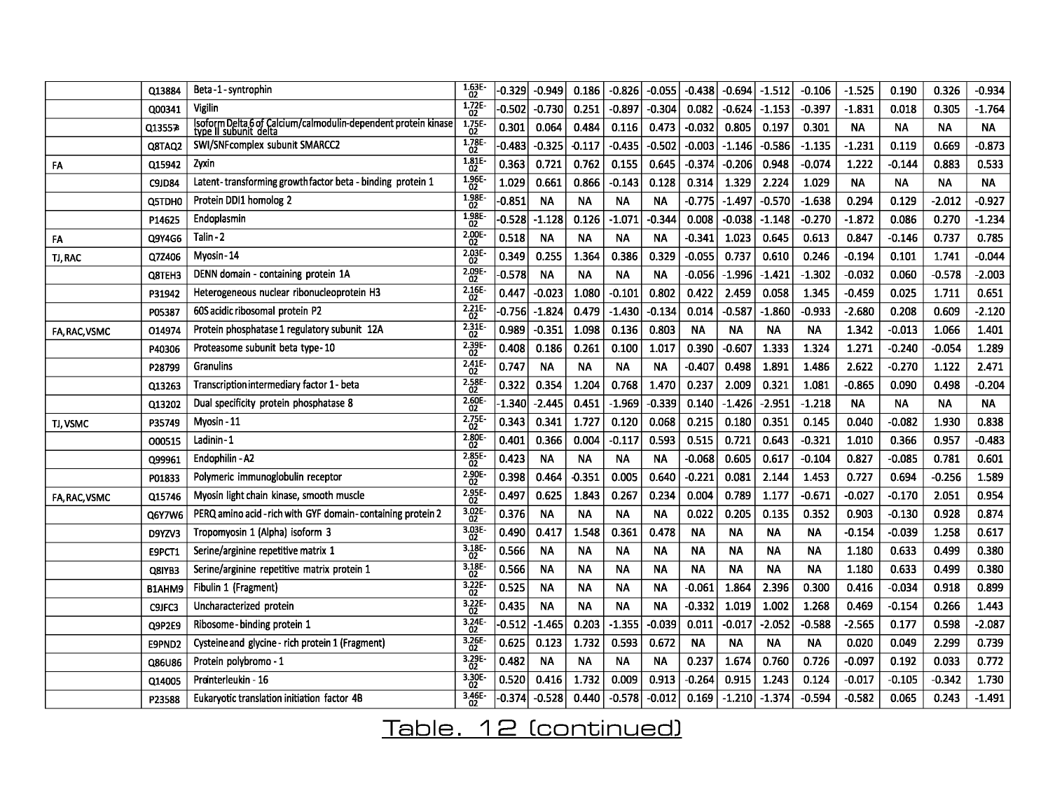

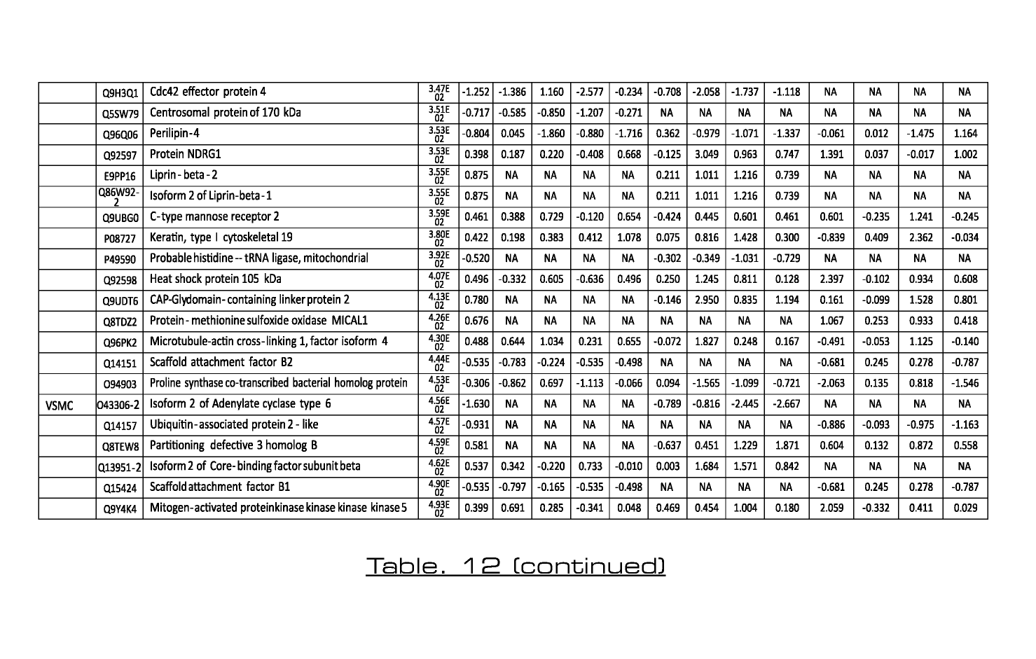

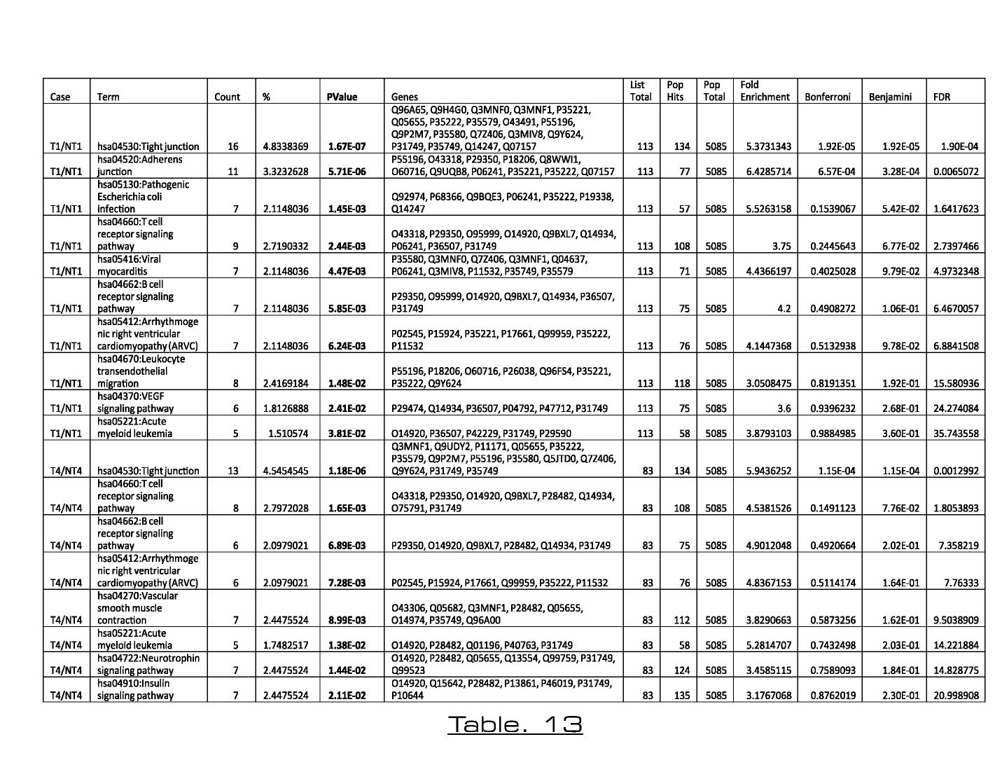

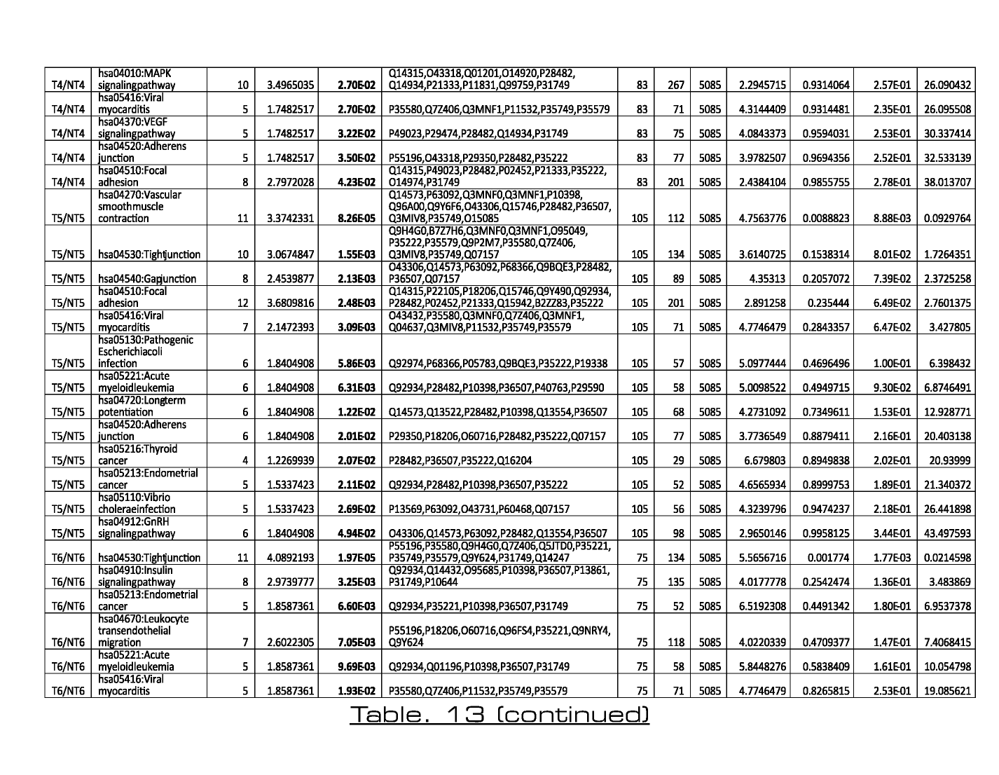

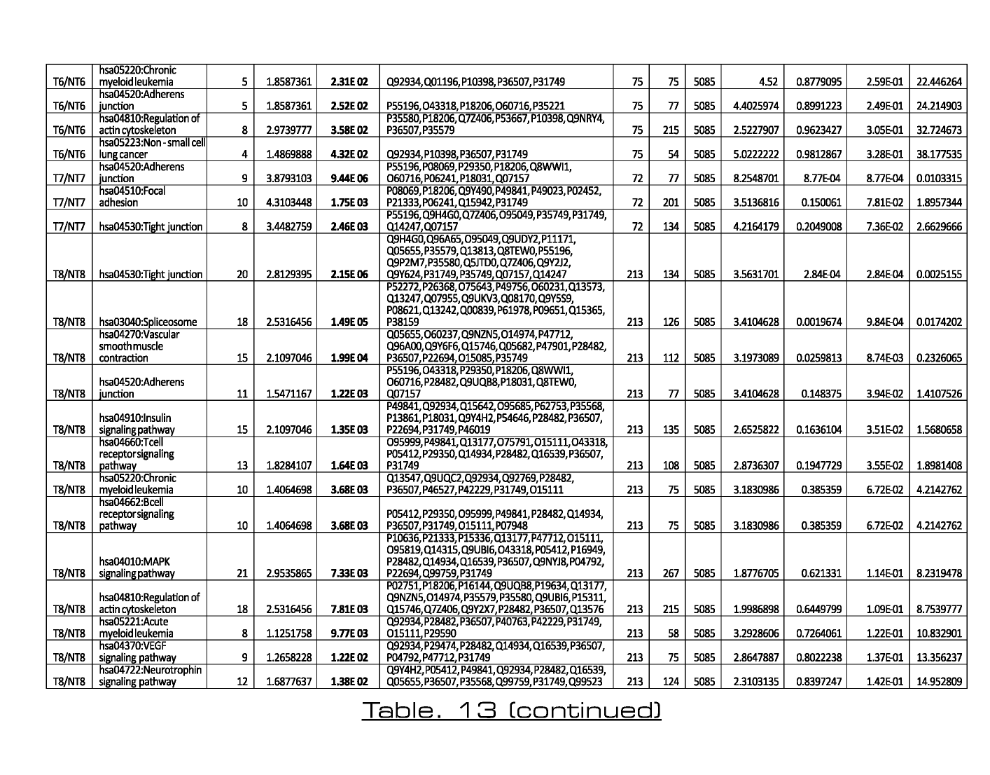

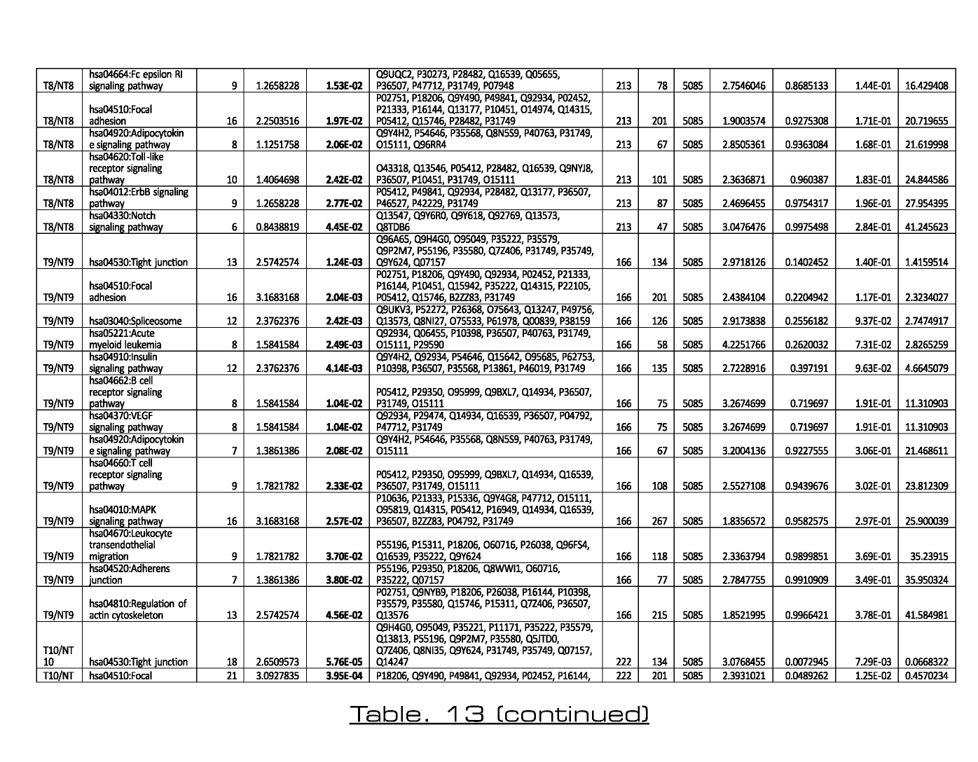

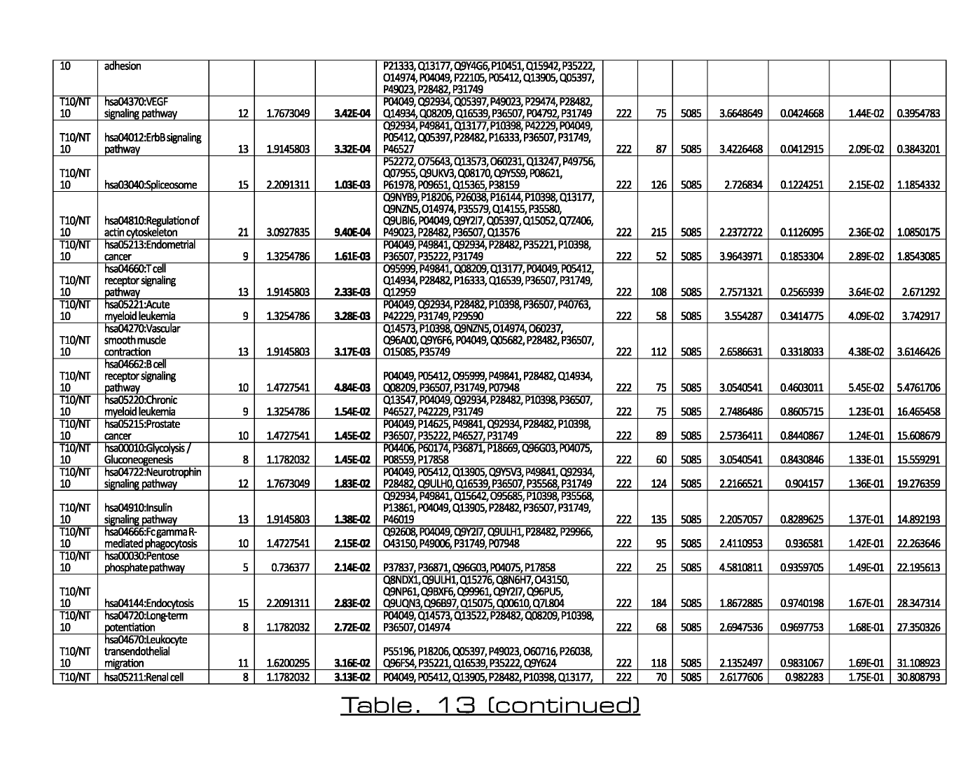

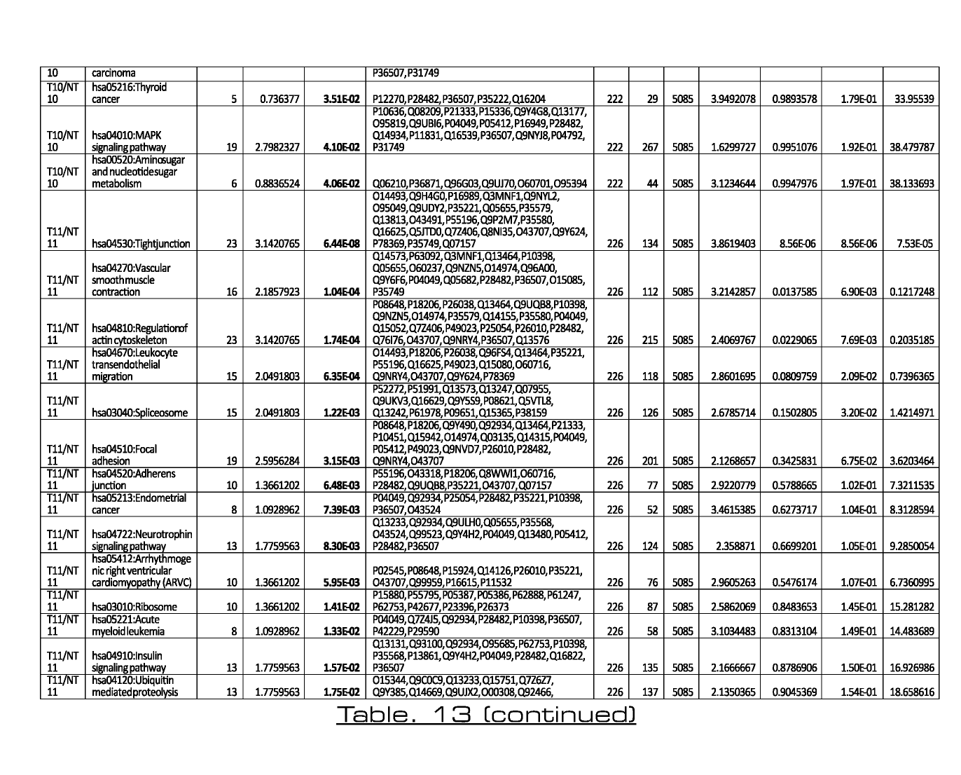

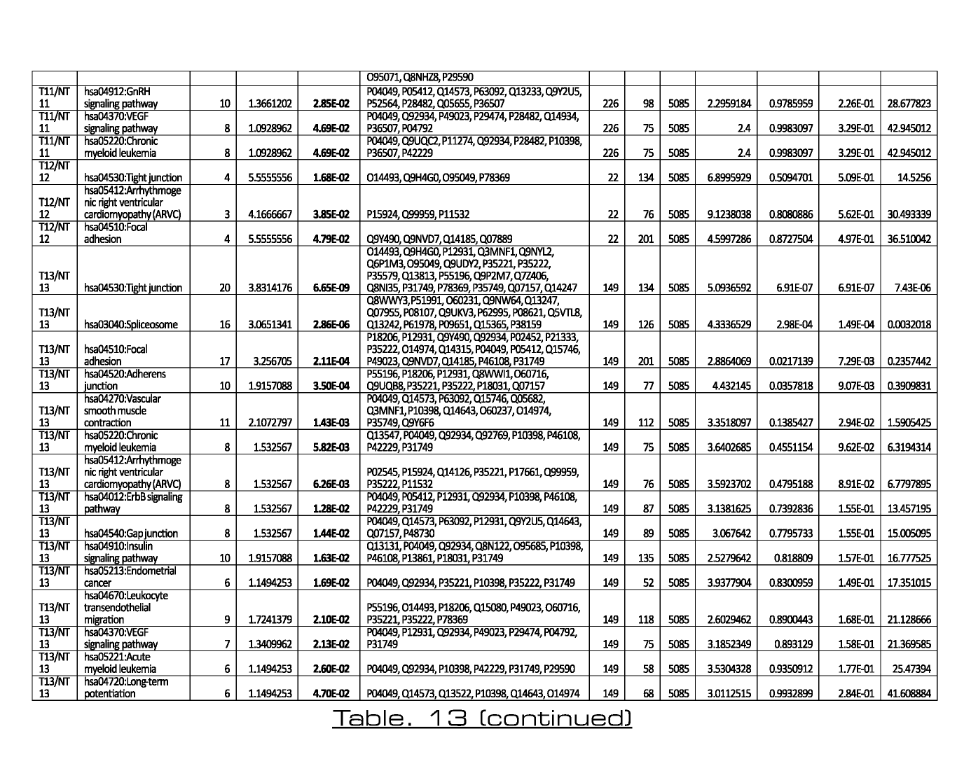

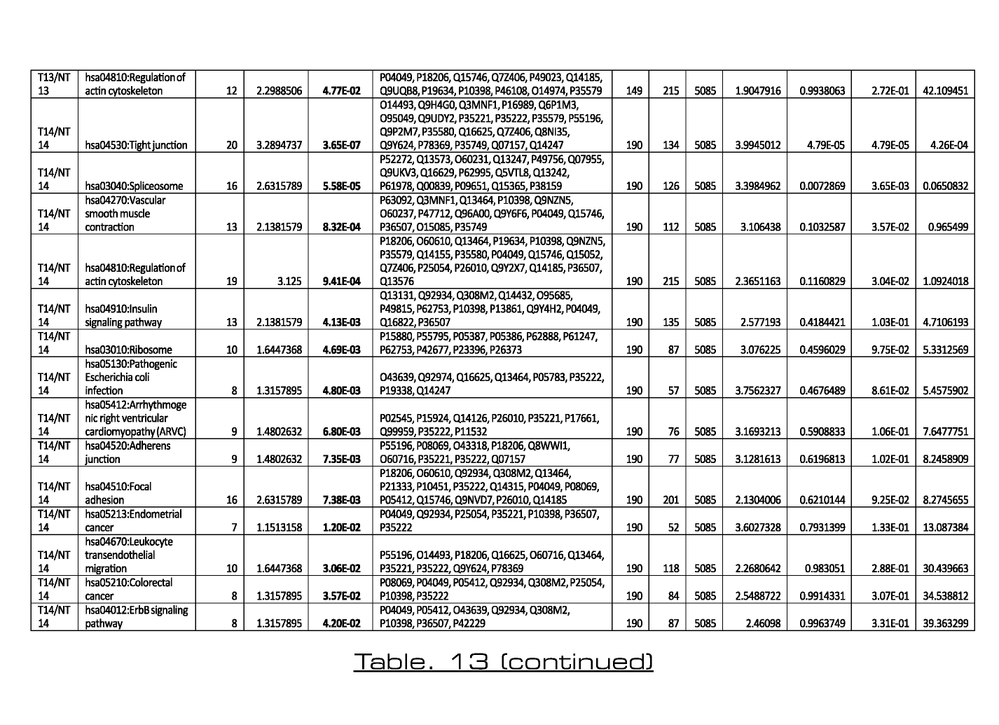

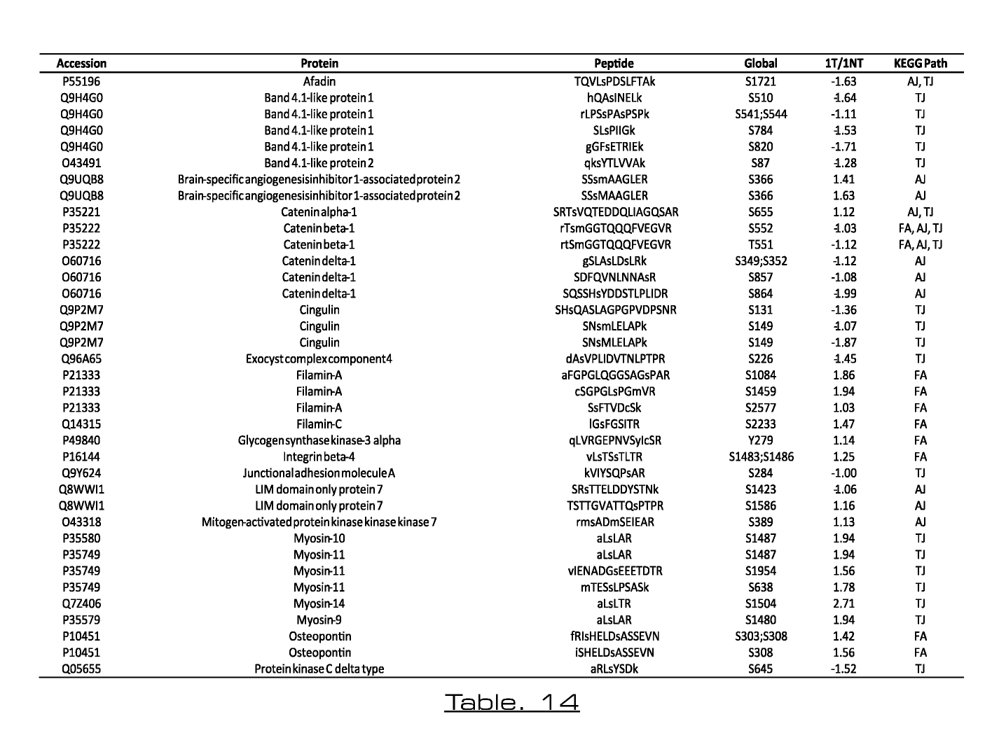

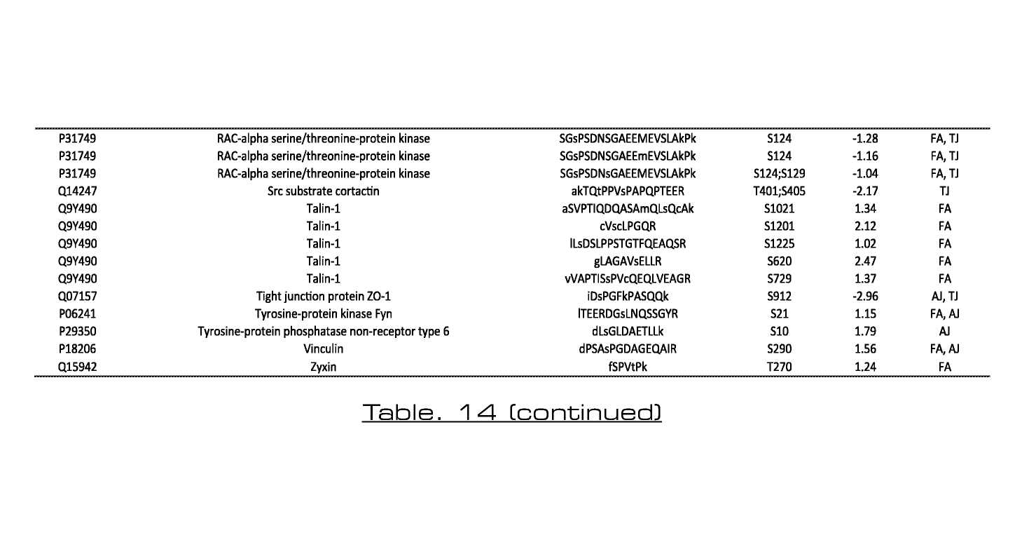

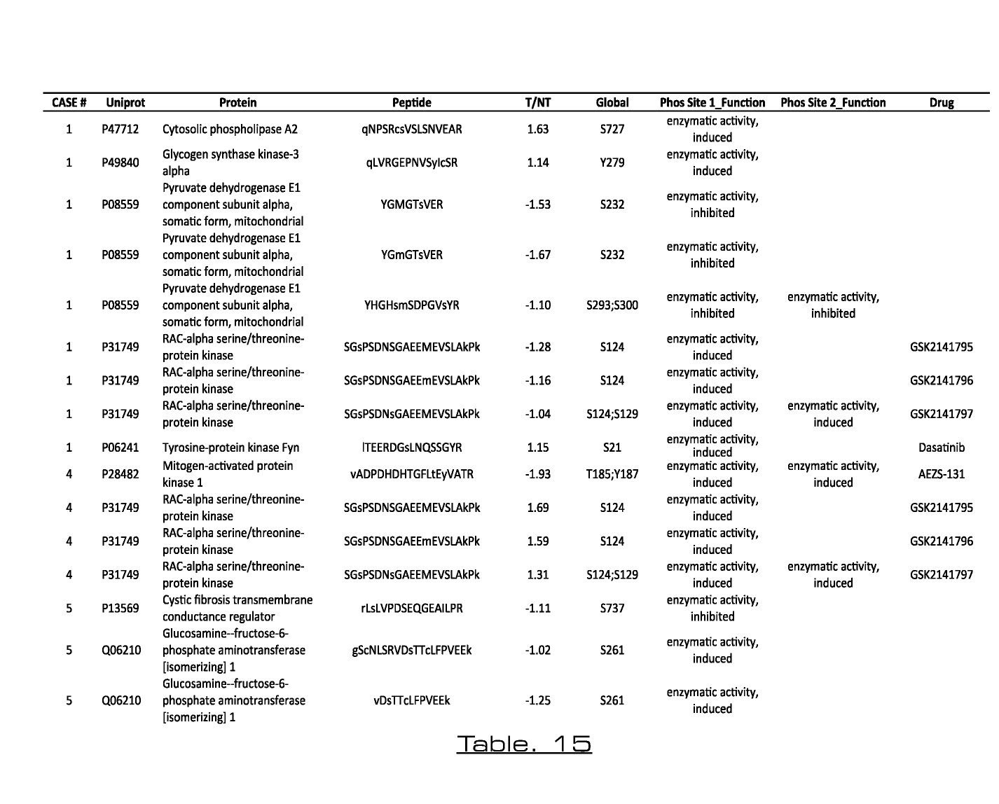

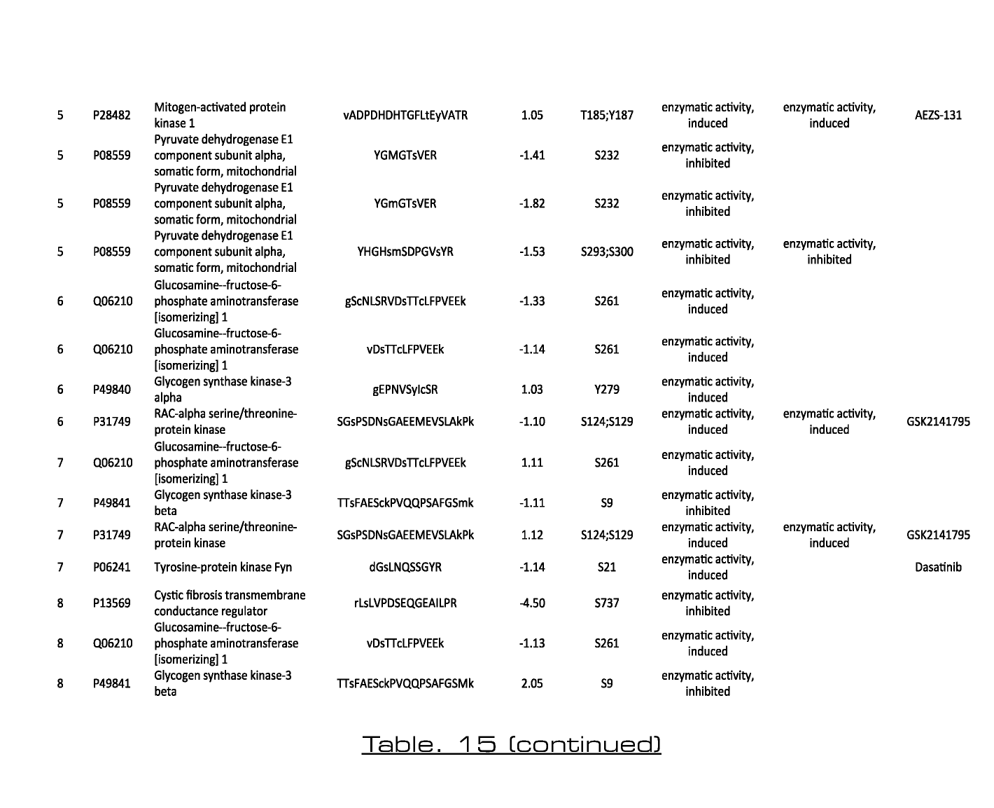

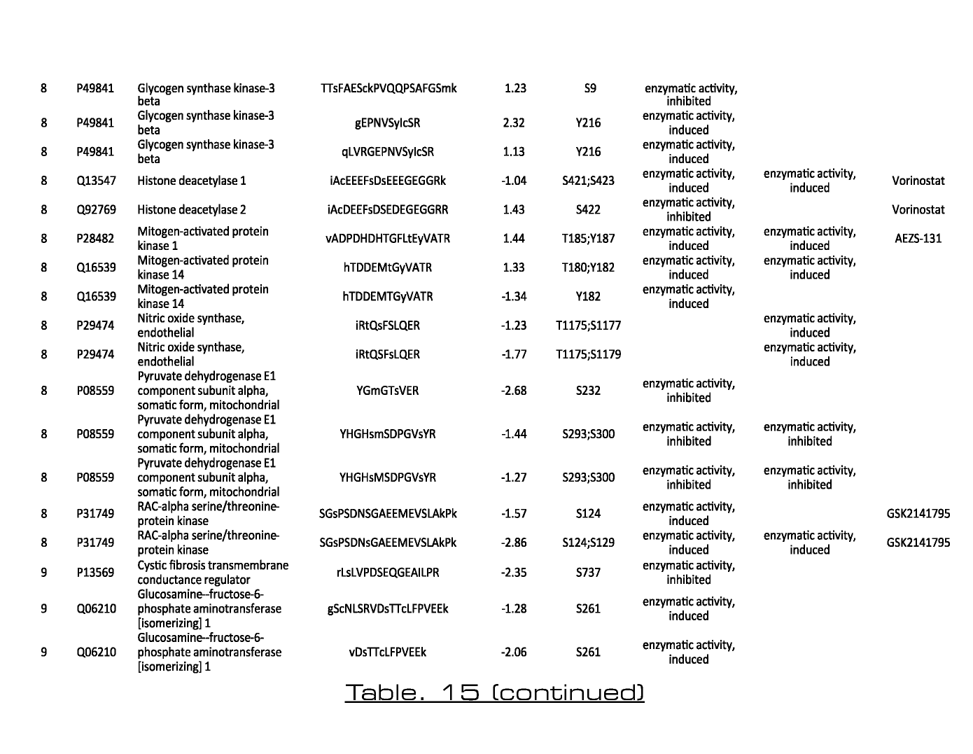

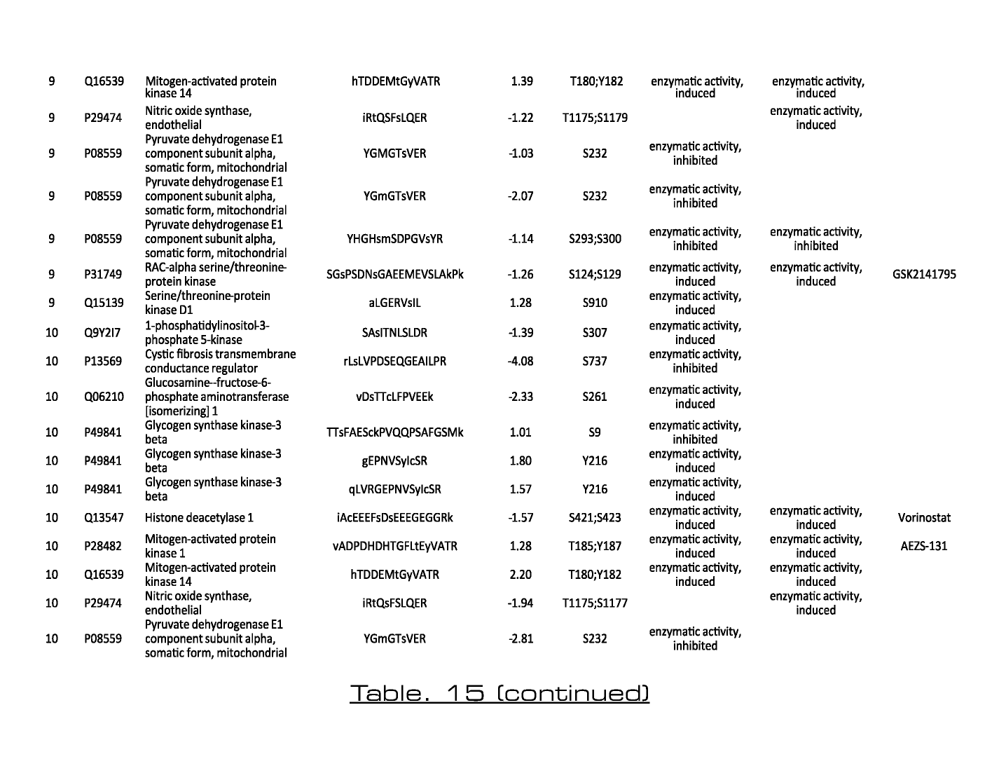

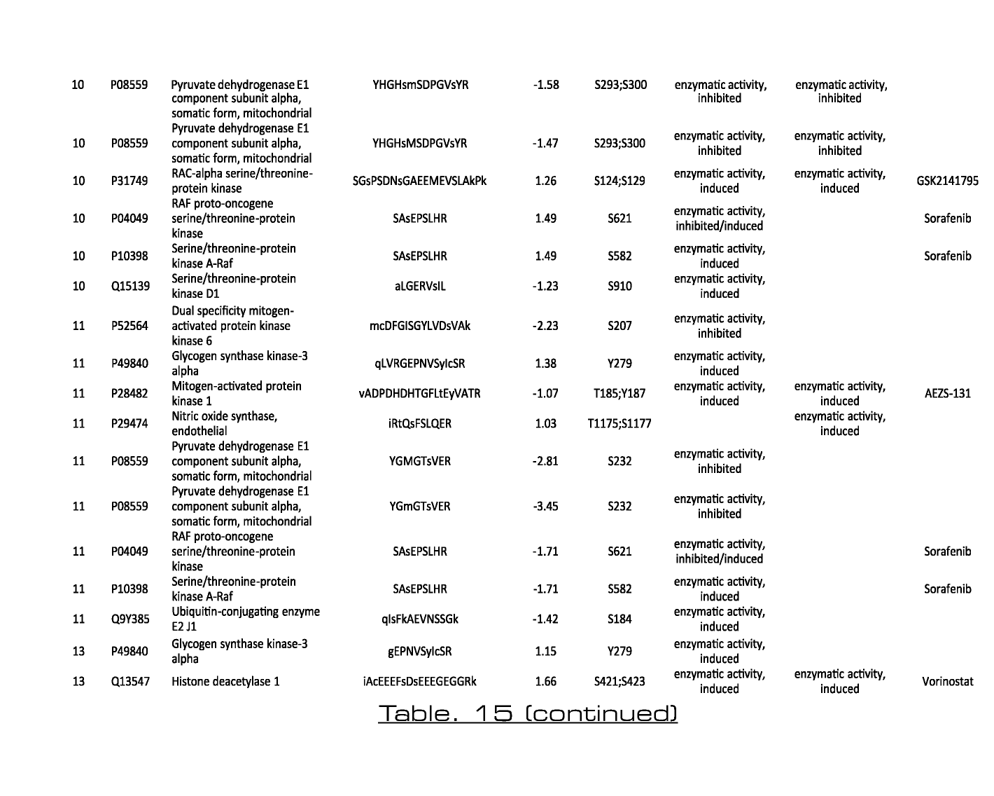

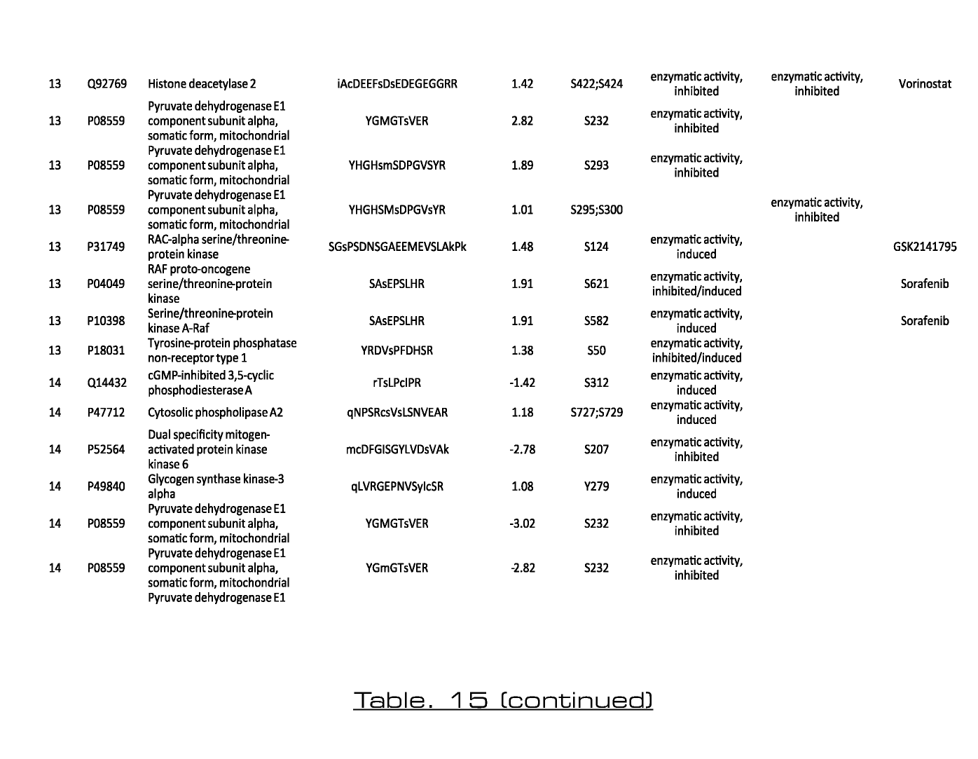

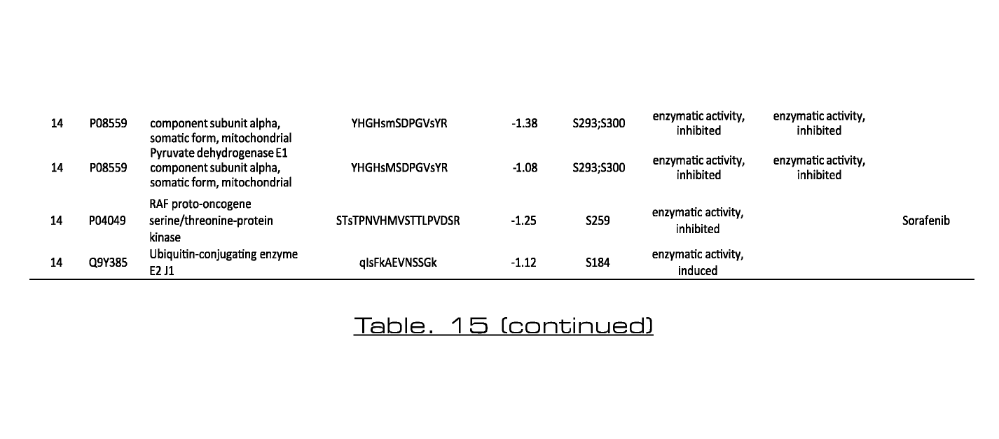

The inventors selected all 408 unique accession numbers of those proteins yielding phosphopeptides (635) with significant differential abundance between tumor compared to non-tumor tissue and uploaded the accession numbers to STRING (Search Tool for the Retrieval of Interacting Genes/Proteins). STRING matched these proteins to the Tight Junction KEGG Signaling pathway with greatest significance giving a p-value of 2.50E-5 after matching 14 of the 408 proteins to the pathway. The inventors also used STRING to identify which GO terms (Biological process, molecular function, and cellular component) these 408 proteins were most strongly associated to. Actin filament based process (n=29; p-value=4.47E-8), Actin binding (n=40; p-value=2.59E-18), and Cytoskeleton (n=77; p-value=2.66E-13) were the GO terms matched with greatest significance. The inventors also used STRING to identify which out of the 408 proteins were associated with the GO biological process ‘Regulation of RAS protein signal transduction’, as RAS is known to be an important onco-protein in pancreatic cancer. 16 of the 408 proteins were matched to this GO biological process with a p-value of 1.06E-2, while 10 of these 16 could be mapped to the STRING network (FIG. 6C).

### Phosphorylation of Protein Kinases

Of particular interest to the inventors was the observation that the phosphopeptides from Serine/Threonine-protein kinase MRCK alpha (see Table 11a) were significantly elevated in tumor compared to non-tumor. This was particulary so for those containing phosphorylation site 51629. MRCK alpha is an important downstream effector of the Rho GTPase, CDC42, and plays a critical role in the regulation of cytoskeleton reorganization, formation of cell protrusion, and promotes cell migration. Further information can be found in Britton et al PLOS ONE March 2014; Vol. 9, Issue 3 e90948, the contents of which are hereby incorporated by reference in their entirety.

Accordingly, MRCK alpha is provided as an important therapeutic target for pancreatic cancer and kinase inhibitors of MRCK alpha as potential therapeutics.

### Case by Case

In addition to determining which proteins and phosphopeptides demonstrated significant differences in abundance between tumor and non-tumor tissue when averaged across all cases, the inventors also wanted to determine which phosphopeptides were highly modulated on a case by case basis. Accession numbers of proteins which yielded phosphopeptides demonstrating log₂ T/NT ratios of ≧1, or ≦−1 (More than 2 fold up/down-regulated), were selected from case 1. These accession numbers were then uploaded to the DAVID Bioinformatic resource which identified KEGG signaling pathways most modulated for case 1. The inventors repeated this approach for each case, then selected KEGG signaling pathways that demonstrated significance, based on p values, and on Benjamini scores on a case by case basis (Table 13). All those KEGG pathways in Table 12 with Benjamini scores ≦0.05 were highlighted in Yellow. Based on p values from the DAVID Bioinformatic output, tight junction signaling pathway was determined to be modulated between tumor compared to non-tumor in all cases (12/12 cases), followed by adherens junction signaling (10/12 cases) and focal adhesion signaling (10/12). FIG. 7, shows the three signaling pathways and the rectangles marked with red stars indicate those proteins the inventors identified as phosphorylated across all 12 cases. Table 3 displays all phosphopeptides displaying significant regulation that belong to proteins involved in Tight Junction and Focal Adhesion signaling pathways, as well as other signaling pathways (Regulation of Actin Cytoskeleton and Vascular smooth muscle contraction) found to be significantly modulated.

Table 14 shows all phosphopeptides demonstrating log₂ T/NT ratios of ≧1, or ≦−1, from case 1, that belong to proteins involved in tight junction, adherens junction, and focal adhesion KEGG signaling pathways. These are also mapped to FIG. 7B

Integrin beta-4—The doubly phosphorylated peptide containing the Integrin beta-4 phosphorylation sites S1483 and S1486, was elevated more than two fold in the tumor tissue compared to non-tumor tissue of case 1. In fact this phosphopeptide was found to be significantly elevated in tumor tissue compared to non-tumor in general across all measured cases (data not shown). Integrin beta-4 phosphorylation has been associated with the disassembly of cell anchoring junctions, such as hemidesmosomes at the trailing edge of migrating cells [32, 33]. Such phosphorylation events have been shown to be induced by Fyn (primarily at Tyrosine residues), PKC (primarily at Serine residues), and other kinases [32].

Catenin alpha-1—The peptide containing Catenin alpha-1 phosphorylation site S655 was elevated more than two fold in tumor tissue compared to non-tumor, in case 1. In fact, the singly phosphorylated peptide containing phospho-5655 was significantly elevated in tumor tissue on average across all cases (Data not shown). Phosphorylation at S641, S655, and S658, was elevated in tumor tissue of all but three cases, two of those three being stage IIA. Interestingly phosphorylation of catenin alpha-1 at S641 has been shown to lead to dissociation between catenin alpha-1 and catenin beta-1 (beta catenin), leading to increased transcriptional activation of beta-catenin and tumor cell invasion [34].

Junctional adhesion molecule A (JAM-A)—The peptide containing JAM-A phosphorylation site S284 was decreased more than two fold in tumor tissue compared to non-tumor, in case 1 and was found to be significantly decreased in tumor tissue compared to non-tumor across all cases (Data not shown). Phosphorylation of JAM-A at S284 is found to be a critical step in the formation and maturation of tight junctions [35]. Here the inventors observe that this phosphorylation event is significantly decreased in tumor tissue an event that could favour epithelial to mesenchymal transition (EMT) of the cells and consequently metastatic spread.

### Phosphorylation Events to Indicate Activity Status of Drug Targets and Other Enzymes

To ascertain relative activation status of enzymes in tumor compared to non-tumor tissue in each case, the inventors used relative abundance of phosphopeptides containing phosphorylation sites known to either induce enzyme activation or inhibition. Table 4 and Table 15 short lists all phosphopeptides displaying log₂ T/NT ratios ≧1 or ≦−1 that contain phosphorylation sites that are known to either induce activation or inhibition of the phosphorylated enzyme, in each case.

Tyrosine-protein kinase Fyn—The relative abundance of the peptide containing phospho-S21 of the Tyrosine-protein kinase Fyn is elevated more than two fold in tumor tissue compared to non-tumor tissue of case 1 (Table 4). Phosphorylation of Fyn at serine 21 is reported to activate Fyn kinase [36]. This suggests therefore, that Fyn is more active in the tumor tissue compared to non-tumor tissue of case 1. Interestingly, phospho-serine 21 of Fyn is detected in all 12 cases, but it is only in case 1 that the inventors observed such relatively high levels in tumor compared to non-tumor. Inversely, the tumor tissue of case 7 shows greater than two fold lower abundance of this phosphopeptide compared to non-tumor tissue. As Fyn is a target of the approved kinase inhibitor Dasatinib this new data suggests that measurement of the peptide containing phospho-S21 using the workflow methods described herein may be an attractive predictive marker for Dasatinib.

Mitogen-activated protein kinase 1 (MAPK1)—The relative abundance of the peptide containing phospho-T185 and phospho-Y187 of the MAPK1 is elevated more than two fold in tumor tissue compared to non-tumor tissue of cases 5, 8, and 10 (Table 4). Phosphorylation of MAPK1 at T185 and/or Y187 is reported to activate MAPK1 [3 7]. This suggests therefore, that MAPK1 is more active in the tumor tissue compared to non-tumor tissue of cases 5, 8, and 10. Inversely, the tumor tissue of cases 4 and 11 shows more than two fold less of this phospho-T185 and phospho-Y187 containing phosphopeptide, compared to non-tumor tissue. MAPK1 is an anti-cancer drug target (AEZS-131 and SCH772984) and is also down-stream of many other anti-cancer drug targets (Anti-HER TKIs, Anti-MEK KIs), therefore this new data suggests that measurement of the peptide containing phospho-T185 and phospho-Y187 using our workflow may be a predictive marker for these targeted anti-cancer therapies. The inventors have also measured the singly phosphorylated peptides containing phospho-T185 or phospho-Y187, as well as the MAPK2 doubly and singly phosphorylated peptides containing phospho-T202 and phospho-Y204. The workflow methods described herein can easily determine whether MAPK2 is phosphorylated on T202 and/or Y204 and/or MAPK1 is phosphorylated on T185, and/or Y187, yielding critical signaling pathway activation status information.

RAC-alpha serine/threonine-protein kinase (AKT1)—The relative abundance of the singly phosphorylated peptides containing phospho-S124 and the doubly phosphorylated peptide containing phospho-S124 and phospho-S129 of AKT1 are elevated more than two fold in tumor tissue compared to non-tumor tissue of cases 4, 7, 10, and 13 (Table 4). Phosphorylation of AKT1 at S124 and/or S129 is reported to activate AKT1 [38, 39]. This suggests that AKT1 is more active in the tumor tissue compared to non-tumor tissue of cases 4, 7, 10, and 13. Therefore, anti-AKT kinase inhibitors may be effective in these patients. Interestingly, Case 10 also demonstrated elevated MAPK1 activity suggesting this patient may be a candidate for dual AKT1 & MAPK1 inhibitor treatment, as such combination strategies have proven efficacy in pancreatic cancer cell lines and xenograft models [12]. Inversely, the relative lower abundance of phosphopeptides containing these activator phosphorylation sites suggests AKT1 is less active in the tumor tissue compared to non-tumor tissue of cases 1, 6, 8, 9, 11, and 14. AKT1 is an anti-cancer drug target therefore, the inventor's data suggests that measurement of the peptides containing phospho-S124 and phospho-S129 using the workflow methods described herein may be an attractive predictive marker for these targeted anti-cancer therapies.

Glycogen synthase kinase-3 alpha—The peptide containing the Glycogen synthase kinase-3 alpha phosphorylation site Y279 increased more than two fold in the tumor tissue compared to non-tumor tissue of cases 1, 6, 13, and 14 (Table 15). Phosphorylation of Y279 causes activation of GSK3a which then induces cell survival, and reduces glycogen production [40]. GSK3a expression was measured in 8 out of 12 cases and shown to be significantly over expressed on average in tumor.

Using the approach where one measures the relative abundance of phosphopeptides containing activator or inhibitor phosphorylation sites, the inventors were able to determine the relative activation status of; Glycogen synthase kinase-3 alpha and beta, Histone deacetylase 1 and 2, RAF proto-oncogene serine/threonine-protein kinase, Serine/threonine-protein kinase A-Raf, Dual specificity mitogen-activated protein kinase kinase 6, Mitogen-activated protein kinase 14, and over 20 others (Table 4 and Table 15).

Notably, the most significantly enriched signalling pathways principally belong to cytoskeletal dynamics and cell adhesion, pathways that are usually deregulated during cell motility and metastatic spreading, highlighting the importance of these proteins in a highly metastatic disease such as pancreatic cancer and demonstrating the validity of the inventors' approach. Many other interesting molecular events, independent of the mentioned KEGG signaling pathways, were also observed in this experiment including the consistent and significant reduction in phosphorylation sites of the Microtubule-associated protein Tau, in all tumor tissue (data not shown), the inverse is known to cause pathology associated with Alzheimer's disease. Also, the activator phosphorylation site, S389 on Casein kinase I isoform epsilon, was significantly elevated on average in tumor tissue.

In conclusion, the inventors provide examples which demonstrate how their LC-MS workflow, can simultaneously measure the abundance and activity of 1000's of signaling and structural proteins in tumor tissue relative to non-tumor tissue, and show how such measurements can be used to better understand the molecular events leading to cancer, and therefore the most suitable inhibitory agents, to treat a patient on a case by case basis. Critically, the inventors have demonstrated using hierarchal clustering of phosphopeptide log₂ T/NT ratios that they can identify those patients more likely to show recurrence at a median follow up of 16.5 months compared to those patients less likely to show recurrence at this time point.

### REFERENCES

[1] Smart J E, Oppermann H, Czernilofsky A P, et al. Characterization of sites for tyrosine phosphorylation in the transforming protein of Rous sarcoma virus (pp60v-src) and its normal cellular homologue (pp60c-src). Proc. Natl. Acad. Sci. 1981; 78:6013-7.
[2] Langer T, Vogtherr M, Elshorst B, et al. NMR backbone assignment of a protein kinase catalytic domain by a combination of several approaches: application to the catalytic subunit of cAMP-dependent protein kinase. Chembiochem. 2004; 5:1508-16.
[3] Chen P L, Scully P, Shew J Y, et al. Phosphorylation of the retinoblastoma gene product is modulated during the cell cycle and cellular differentiation. Cell. 1989; 58:1193-8.
[4] Bononi A, Agnoletto C, De Marchi E, et al. Protein kinases and phosphatases in the control of cell fate. Enzyme Res. 2011; 2011:3290.
[5] Bond-Smith G, Banga N, Hammond T_{M}, et al. Pancreatic adenocarcinoma. BMJ. 2012; 344:e2476.
[6] Michl P, Gress T_{M}. Current concepts and novel targets in advanced pancreatic cancer. Gut. 2013; 62:317-26.
[7] Llovet J M, Ricci S, Mazzaferro V, et al; SHARP Investigators Study Group. Sorafenib in advanced hepatocellular carcinoma. N Engl J Med. 2008; 359:378-90.
[8] Engholm-Keller K, Larsen M R. Technologies and challenges in large-scale phosphoproteomics. Proteomics. 2013; 13:910-31.
[9] Mann M, Kulak N A, Nagaraj N, et al. The coming age of complete, accurate, and ubiquitous proteomes. Mol Cell. 2013; 49:583-90.
[10] McAlister G C, Huttlin E L, Haas W, et al. Increasing the multiplexing capacity of TMTs using reporter ion isotopologues with isobaric masses. Anal Chem. 2012; 84:7469-78.
[11] Werner T, Becher I, Sweetman G, et al. High-resolution enabled TMT 8-plexing. Anal Chem. 2012; 84:7188-94.
[12] Craig D. Logsdon & Marina Pasca di Magliano, Roles for KRAS in Pancreatic Tumor Development and Progression, GASTROENTEROLOGY. 2013; 144:1220-1229.
[13] Yang W, Xia Y, Hawke D, et al. PKM2 phosphorylates histone H3 and promotes gene transcription and tumorigenesis. Cell. 2012; 150:685-96.
[14] Christofk H R, Vander Heiden M G, Harris M H, et al. The M2 splice isoform of pyruvate kinase is important for cancer metabolism and tumour growth. Nature. 2008; 452:230-3.
[15] Kondo S, Lu Y, Debbas M, Lin A W, et al. Characterization of cells and gene-targeted mice deficient for the p53-binding kinase homeodomain-interacting protein kinase 1 (HIPK1). Proc Natl Acad Sci USA. 2003; 100:5431-6.
[16] Lee D, Park S J, Sung K S, et al. Mdm2 associates with Ras effector NORE1 to induce the degradation of oncoprotein HIPK1. EMBO Rep. 2012; 13:163-9.
[17] Zhao L, Wang H, Liu C, et al. Promotion of colorectal cancer growth and metastasis by the LIM and SH3 domain protein 1. Gut. 2010; 59:1226-35.
[18] Grunewald T G, Kammerer U, Winkler C, et al. Overexpression of LASP-1 mediates migration and proliferation of human ovarian cancer cells and influences zyxin localisation. Br J Cancer. 2007; 96:296-305.
[19] Zhang Y, Ye Y, Shen D, et al. Identification of transgelin-2 as a biomarker of colorectal cancer by laser capture microdissection and quantitative proteome analysis. Cancer Sci. 2010; 101:523-9.
[20] Morohashi Y, Balklava Z, Ball M, et al. Phosphorylation and membrane dissociation of the ARF exchange factor GBF1 in mitosis.

Biochem J. 2010; 427:401-12.
[21] Miyasaka K Y, Kida Y S, Sato T, et al. Csrpl regulates dynamic cell movements of the mesendoderm and cardiac mesoderm through interactions with Dishevelled and Diversin. Proc Natl Acad Sci USA. 2007; 104:11274-9.
[22] Hirasawa Y, Arai M, Imazeki F, et al. Methylation status of genes upregulated by demethylating agent 5-aza-2′-deoxycytidine in hepatocellular carcinoma. Oncology. 2006; 71:77-85.
[23] Goicoechea S M, Bednarski B, Garcia-Mata R, et al. Palladin contributes to invasive motility in human breast cancer cells. Oncogene. 2009; 28:587-98.
[24] Weitzdoerfer R, Fountoulakis M, Lubec G. Aberrant expression of dihydropyrimidinase related proteins-2, -3 and -4 in fetal Down syndrome brain. J Neural Transm Suppl. 2001; 61:95-107.
[25] Jung C R, Lim J H, Choi Y, et al. Enigma negatively regulates p53 through MDM2 and promotes tumor cell survival in mice. J Clin Invest. 2010; 120:4493-506.
[26] Haynes J, Srivastava J, Madson N, et al. Dynamic actin remodeling during epithelial-mesenchymal transition depends on increased moesin expression. Mol Biol Cell. 2011; 22:4750-64.
[27] Yonezawa S, Higashi M, Yamada N, et al. Mucins in human neoplasms: clinical pathology, gene expression and diagnostic application. Pathol Int. 2011; 61:697-716.
[28] Wei X, Xu H, Kufe D. Human mucin 1 oncoprotein represses transcription of the p53 tumor suppressor gene. Cancer Res. 2007; 67:1853-8.
[29] Ren J, Li Y, Kufe D. Protein kinase C delta regulates function of the DF3/MUC1 carcinoma antigen in beta-catenin signaling. J Biol Chem. 2002; 277:17616-22.
[30] Schwappacher R, Rangaswami H, Su-Yuo J, et al. cGMP-dependent protein kinase Iβ regulates breast cancer cell migration and invasion via a novel interaction with the actin/myosin-associated protein caldesmon. J Cell Sci. 2013. [Epub ahead of print].
[31] Mayanagi T, Morita T, Hayashi K, et al. Glucocorticoid receptor-mediated expression of caldesmon regulates cell migration via the reorganization of the actin cytoskeleton. J Biol Chem. 2008; 283:31183-96.
[32] Germain E C, Santos T M, Rabinovitz I. Phosphorylation of a novel site on the {beta}4 integrin at the trailing edge of migrating cells promotes hemidesmosome disassembly. Mol Biol Cell. 2009; 20:56-67.
[33] Dans M, Gagnoux-Palacios L, Blaikie P, et al. Tyrosine phosphorylation of the beta 4 integrin cytoplasmic domain mediates Shc signaling to extracellular signal-regulated kinase and antagonizes formation of hemidesmosomes. J Biol Chem. 2001; 276:1494-502.
[34] Ji H, Wang J, Nika H, et al. EGF-induced ERK activation promotes CK2-mediated disassociation of alpha-Catenin from beta-Catenin and transactivation of beta-Catenin. Mol Cell. 2009; 36:547-59.
[35] Iden S, Misselwitz S, Peddibhotla S S, et al. aPKC phosphorylates JAM-A at Ser285 to promote cell contact maturation and tight junction formation. J Cell Biol. 2012; 196:623-39.
[36] Yeo M G, Oh H J, Cho H S, et al. Phosphorylation of Ser 21 in Fyn regulates its kinase activity, focal adhesion targeting, and is required for cell migration. J Cell Physiol. 2011; 226:236-47.
[37] Schramek H, Schumacher M, Wilflingseder D, et al. Differential expression and activation of MAP kinases in dedifferentiated MDCK-focus cells. Am J Physiol. 1997; 272:C383-91.
[38] Bellacosa A, Chan T O, Ahmed N N, et al. Akt activation by growth factors is a multiple-step process: the role of the PH domain. Oncogene. 1998; 17:313-25.
[39] Di Maira G, Salvi M, Arrigoni G, et al. Protein kinase CK2 phosphorylates and upregulates Akt/PKB. Cell Death Differ. 2005; 12:668-77.
[40] Kotliarova S, Pastorino S, Kovell L C, et al. Glycogen synthase kinase-3 inhibition induces glioma cell death through c-MYC, nuclear factor-kappaB, and glucose regulation. Cancer Res2008; 68:6643-51.
[41] Wang H, Chang-Wong T, Tang H Y, et al. Comparison of Extensive Protein Fractionation and Repetitive LC-MS/MS Analyses on Depth of Analysis for Complex Proteomes. J Proteome Res. 2010; 9: 1032-40.

1-74. (canceled)

75. A method of selecting a treatment regime for a subject suffering from pancreatic cancer, said method comprising the steps of:
(1) determining protein expression levels and/or protein phosphorylation levels of a plurality of proteins in a pancreatic tumour sample of said subject so as to produce an expression level and/or protein phosphorylation profile of said tumour;
(2) comparing said tumour profile with a reference profile, said reference profile being representative of pancreatic tumour phenotypes selected from tumour, non-tumour, recurrence, non-recurrence, drug susceptibility, primary tumour and/or secondary (metastatic) tumour;
(3) classifying the pancreatic tumour of the subject into a phenotype based on the comparison between the tumour profile and the reference profile; and
(4) selecting a treatment regime according to phenotype of the pancreatic tumour of the subject;
wherein the plurality of proteins are selected from a biomarker panel represented by Tables 2, 3, 4, 11A, 11B, 12, 13 and/or 15, preferably, Table 15 and/or Table 4.

76. A method according to claim 75, wherein the plurality of proteins is selected from the group consisting of Tyrosine-protein kinase (Fyn), Tyrosine-protein kinase CSK (Src), RAF proto-oncogene serine/threonine-protein kinase, Histone deacetylase 1, Histone deacetylase 2, Rapamucin-insensitive companion of mTOR (RICTOR); ERK1 mitogen-activated protein kinase, ERK2 mitogen-activated protein kinase, RAC-alpha serine/threonine-protein kinase, Integrin Beta-4, Catenin alpha-1, Junctional adhesion molecule A (JAM-A), Tyrosine protein kinase Fyn, Mitogen-activated protein kinase 1 (MAPK1), RAC-alpha serine/threonine-protein kinase (AKT1), and Glycogen synthase kinase-3 alpha.

77. A method according to claim 75, wherein the plurality of proteins are selected from Table 15 and/or Table 4 and the treatment regime is selected based on the determination of drug susceptibility phenotype characterised by the increase or decrease in phosphorylation levels of tyrosine-protein kinase Fyn, Mitogen-activated protein kinase 1 (MAPK1), Mitogen-activated protein kinase 3 (MAPK3); RAC-alpha serine/threonine-protein kinase (AKT1) and/or Glycogen synthase kinase-3 alpha.

78. A method according to claim 75, wherein the treatment regime comprises administering a drug selected from the group consisting of Dasatinib, Sorafenib, Vorinostat, Temsirolimus, AEZS-131 and GSK2141795.

79. A method according to claim 75, wherein the step of determining protein expression levels or protein phosphorylation levels of the plurality of proteins in said sample is performed using an antibody or antibody fragment capable of specifically binding to said protein, preferably, wherein said antibody or antibody fragment is capable of specifically binding to a phosphorylated site on said protein.

80. A method according to claim 75, wherein the step of determining protein expression levels or protein phosphorylation levels of the plurality of proteins in said sample is performed by mass spectrometry.

81. A method according to claim 75, wherein said step of determining protein expression levels or protein phosphorylation levels of the plurality of proteins is performed by Selected Reaction Monitoring using one or more transitions for protein derived peptides or phosphopetides; and comparing the peptide or phosphopeptide levels in the sample being tested with reference peptide or phosphopeptide levels previously determined to represent a molecular phenotype.

82. A method according to claim 75, wherein said step of comparing the tumour profile with a reference profile comprises comparing an amount of protein-derived peptides from the pancreatic sample with known amounts of corresponding synthetic peptides, wherein the synthetic peptides are identical in sequence to the protein-derived peptides obtained from the sample except for a label, wherein the label is, preferably, a tag of a different mass or a heavy isotope.

83. A solid support comprising a plurality of binding members, each capable of specifically and selectively binding to one of a plurality of proteins or nucleic acid sequences encoding said proteins; wherein said proteins are selected from Tables 2, 3, 4, 11A, 11B, 12, 13 and/or 15.

84. A synthetic peptide or a plurality of synthetic peptides each having a sequence identical to a fragment of one of a plurality of marker proteins selected from Tables 2, 3, 4, 11A, 11B, 12, 13 and/or 15, said fragment resulting from digestion of the protein by trypsin, ArgC, AspN or Lys-C digestion, and preferably wherein said plurality of marker proteins includes at least one marker protein selected from the group consisting of Homeodomain-interacting protein kinase 1 (HIPK1); Serine/threonine-protein kinase MRCK alpha (MRCK alpha); and Myosin light chain kinase, smooth muscle (MLCK).

85. A synthetic peptide according to claim 84, further comprising a label, preferably, a heavy isotope.

86. A method of predicting susceptibility of a pancreatic tumour to a treatment, said method comprising the steps of:
(1) determining protein expression levels and/or protein phosphorylation levels of a plurality of proteins in a pancreatic tumour sample obtained from said subject, so as to produce an expression level and/or protein phosphorylation profile of said tumour;
(2) comparing said tumour profile with a reference profile, said reference profile being representative of pancreatic tumour phenotypes selected from tumour, non-tumour, recurrence, non-recurrence, drug susceptibility, primary tumour and/or secondary (metastatic) tumour;
(3) classifying the pancreatic tumour of the subject into a phenotype based on the comparison between the tumour profile and the reference profile; and
(4) determining susceptibility of the pancreatic tumour to said treatment;
wherein the plurality of proteins are selected from a biomarker panel represented by Tables 2, 3, 4, 11A, 11B, 12, 13 and/or 15.

87. A method according to claim 86, wherein the treatment is selected from the group consisting of treatment with Dasatinib, Sorafenib, Vorinostat, Temsirolimus, AEZS-131 and GSK2141795.

88. A method according to claim 86, wherein the plurality of proteins is selected from the group consisting of Tyrosine-protein kinase (Fyn), Tyrosine-protein kinase CSK (Src), RAF proto-oncogene serine/threonine-protein kinase, Histone deacetylase 1, Histone deacetylase 2, Rapamucin-insensitive companion of mTOR (RICTOR); ERK1 mitogen-activated protein kinase, ERK2 mitogen-activated protein kinase, RAC-alpha serine/threonine-protein kinase, Integrin Beta-4, Catenin alpha-1, Junctional adhesion molecule A (JAM-A), Tyrosine protein kinase Fyn, Mitogen-activated protein kinase 1 (MAPK1), RAC-alpha serine/threonine-protein kinase (AKT1), and Glycogen synthase kinase-3 alpha.

89. A method according to claim 86, wherein:
(a) the determination step (1) comprises determining the level of phospho-S21 on Tyrosine-protein kinase Fyn and the treatment is treatment with Dasatinib (BMS-354825—Sprycel™), wherein an up-regulation of this protein is indicative that the pancreatic tumour is susceptible to treatment with Dasatinib; or
(b) the determination step (1) comprises determining the level of phospho-T185 and/or Y187 on Mitogen-activated protein kinase 1 (MAPK1) and the treatment is treatment with AEZS-131 (Aeterna Zentaris, Inc.) and/or SCH772984 (Merck), wherein an up-regulation of this protein is indicative that the pancreatic tumour will be susceptible to treatment with AEZS-131 and/or SCH772984.

90. A method of treating a subject having pancreatic cancer; said method comprising administering a kinase inhibitor capable of inhibiting the activity of a protein kinase selected from the group consisting of Homeodomain-interacting protein kinase 1 (HIPK1); Serine/threonine-protein kinase MRCK alpha (MRCK alpha); and Myosin light chain kinase, smooth muscle (MLCK).


SEQUENCE LISTING (182 sequences)

SEQ ID NO: 1 - protein, 9 aa, Homo sapiens
LNQPGTPTR

SEQ ID NO: 2 - protein, 12 aa, Homo sapiens
LNQPGTPTRTAV

SEQ ID NO: 3 - protein, 12 aa, Homo sapiens
TLRLNQPGTPTR

SEQ ID NO: 4 - protein, 11 aa, Homo sapiens
DGSLNQSSGYR

SEQ ID NO: 5 - protein, 16 aa, Homo sapiens
LTEERDGSLNQSSGYR

SEQ ID NO: 6 - protein, 10 aa, Homo sapiens
LIEDNEYTAR

SEQ ID NO: 7 - protein, 16 aa, Homo sapiens
LFGGFNSSDTVTSPQR

SEQ ID NO: 8 - protein, 13 aa, Homo sapiens
GAQASSGSPALPR

SEQ ID NO: 9 - protein, 19 aa, Homo sapiens
STSTPNVHMVSTTLPVDSR

SEQ ID NO: 10 - protein, 9 aa, Homo sapiens
SASEPSLHR

SEQ ID NO: 11 - protein, 9 aa, Homo sapiens
SASEPSLNR

SEQ ID NO: 12 - protein, 9 aa, Homo sapiens
ISICSSDKR

SEQ ID NO: 13 - protein, 19 aa, Homo sapiens
IACEEEFSDSEEEGEGGRK

SEQ ID NO: 14 - protein, 19 aa, Homo sapiens
IACDEEFSDSEDEGEGGRR

SEQ ID NO: 15 - protein, 17 aa, Homo sapiens
HIEDTGSTPSIGENDLK

SEQ ID NO: 16 - protein, 9 aa, Homo sapiens
SVSSYGNIR

SEQ ID NO: 17 - protein, 19 aa, Homo sapiens
IADPEHDHTGFLTEYVATR

SEQ ID NO: 18 - protein, 19 aa, Homo sapiens
VADPDHDHTGFLTEYVATR

SEQ ID NO: 19 - protein, 21 aa, Homo sapiens
SGSPSDNSGAEEMEVSLAKPK

SEQ ID NO: 20 - protein, 14 aa, Homo sapiens
STSQGSINSPVYSR

SEQ ID NO: 21 - protein, 9 aa, Homo sapiens
QGMSPTFSR

SEQ ID NO: 22 - protein, 13 aa, Homo sapiens
SNSDESNFSEKLR

SEQ ID NO: 23 - protein, 7 aa, Homo sapiens
LGSLSAR

SEQ ID NO: 24 - protein, 16 aa, Homo sapiens
LGSLSARSDSEATISR

SEQ ID NO: 25 - protein, 9 aa, Homo sapiens
STSLSALVR

SEQ ID NO: 26 - protein, 8 aa, Homo sapiens
QVSLPVTK

SEQ ID NO: 27 - protein, 14 aa, Homo sapiens
ATSLPSLDTPGELR

SEQ ID NO: 28 - protein, 9 aa, Homo sapiens
LNQPGTPTR

SEQ ID NO: 29 - protein, 12 aa, Homo sapiens
LNQPGTPTRTAV

SEQ ID NO: 30 - protein, 10 aa, Homo sapiens
AISSANLLVR

SEQ ID NO: 31 - protein, 12 aa, Homo sapiens
CSGPGLSPGMVR

SEQ ID NO: 32 - protein, 9 aa, Homo sapiens
LGSFGSITR

SEQ ID NO: 33 - protein, 14 aa, Homo sapiens
SLDSPTSSPGAGTR

SEQ ID NO: 34 - protein, 12 aa, Homo sapiens
SAPPSPPPPGTR

SEQ ID NO: 35 - protein, 13 aa, Homo sapiens
SEGLPSECRSVTD

SEQ ID NO: 36 - protein, 9 aa, Homo sapiens
TYSLTTPAR

SEQ ID NO: 37 - protein, 12 aa, Homo sapiens
RGSLPAEASCTT

SEQ ID NO: 38 - protein, 13 aa, Homo sapiens
TSSEDNLYLAVLR

SEQ ID NO: 39 - protein, 18 aa, Homo sapiens
SSSVGSSSSYPISPAVSR

SEQ ID NO: 40 - protein, 12 aa, Homo sapiens
SLSPSSNSAFSR

SEQ ID NO: 41 - protein, 17 aa, Homo sapiens
QSSATSSFGGLGGGSVR

SEQ ID NO: 42 - protein, 14 aa, Homo sapiens
QRSAPDLKESGAAV

SEQ ID NO: 43 - protein, 11 aa, Homo sapiens
NLALSRESLVV

SEQ ID NO: 44 - protein, 10 aa, Homo sapiens
VLTAKASTDL

SEQ ID NO: 45 - protein, 6 aa, Homo sapiens
ALSLTR

SEQ ID NO: 46 - protein, 10 aa, Homo sapiens
FGTFGGLGSK

SEQ ID NO: 47 - protein, 20 aa, Homo sapiens
IASDEEIQGTKDAVIQDLER

SEQ ID NO: 48 - protein, 18 aa, Homo sapiens
SSSVGSSSSYPISPAVSR

SEQ ID NO: 49 - protein, 12 aa, Homo sapiens
TSSEPEFNSLPR

SEQ ID NO: 50 - protein, 14 aa, Homo sapiens
SGAQASSTPLSPTR

SEQ ID NO: 51 - protein, 9 aa, Homo sapiens
LSPSPTSQR

SEQ ID NO: 52 - protein, 17 aa, Homo sapiens
SVGGSGGGSFGDNLVTR

SEQ ID NO: 53 - protein, 15 aa, Homo sapiens
VSPATPGPALSDILR

SEQ ID NO: 54 - protein, 8 aa, Homo sapiens
SMSLTLGK

SEQ ID NO: 55 - protein, 14 aa, Homo sapiens
TASGSSVTSLDGTR

SEQ ID NO: 56 - protein, 11 aa, Homo sapiens
AEGVSTPLAGR

SEQ ID NO: 57 - protein, 10 aa, Homo sapiens
TYSDEANQMR

SEQ ID NO: 58 - protein, 12 aa, Homo sapiens
SPSFGAGEGLLR

SEQ ID NO: 59 - protein, 12 aa, Homo sapiens
ILSQSTDSLNMR

SEQ ID NO: 60 - protein, 6 aa, Homo sapiens
NLSLVR

SEQ ID NO: 61 - protein, 11 aa, Homo sapiens
SMSASSGLSAR

SEQ ID NO: 62 - protein, 9 aa, Homo sapiens
AQISSPNLR

SEQ ID NO: 63 - protein, 8 aa, Homo sapiens
SLSVLSPR

SEQ ID NO: 64 - protein, 13 aa, Homo sapiens
SGSLGQPSPSAQR

SEQ ID NO: 65 - protein, 9 aa, Homo sapiens
IDISPSTFR

SEQ ID NO: 66 - protein, 11 aa, Homo sapiens
SGEGEVSGLMR

SEQ ID NO: 67 - protein, 7 aa, Homo sapiens
CVSALGR

SEQ ID NO: 68 - protein, 14 aa, Homo sapiens
LRNSLDSSDSDSAL

SEQ ID NO: 69 - protein, 15 aa, Homo sapiens
ALSSDSILSPAPDAR

SEQ ID NO: 70 - protein, 14 aa, Homo sapiens
SLYASSPGGVYATR

SEQ ID NO: 71 - protein, 7 aa, Homo sapiens
SSVPGVR

SEQ ID NO: 72 - protein, 12 aa, Homo sapiens
MYPESTTGSPAR

SEQ ID NO: 73 - protein, 13 aa, Homo sapiens
DEILPTTPISEQK

SEQ ID NO: 74 - protein, 11 aa, Homo sapiens
RLSSLRASTSK

SEQ ID NO: 75 - protein, 17 aa, Homo sapiens
SRSPGSPVGEGTGSPPK

SEQ ID NO: 76 - protein, 9 aa, Homo sapiens
HQASINELK

SEQ ID NO: 77 - protein, 10 aa, Homo sapiens
GGFSETRIEK

SEQ ID NO: 78 - protein, 12 aa, Homo sapiens
AEEDEILNRSPR

SEQ ID NO: 79 - protein, 11 aa, Homo sapiens
GSLASLDSLRK

SEQ ID NO: 80 - protein, 14 aa, Homo sapiens
DQTASAPATPLVNK

SEQ ID NO: 81 - protein, 8 aa, Homo sapiens
SMSFQGIR

SEQ ID NO: 82 - protein, 15 aa, Homo sapiens
GLPSPYNMSSAPGSR

SEQ ID NO: 83 - protein, 12 aa, Homo sapiens
SRSPLELEPEAK

SEQ ID NO: 84 - protein, 14 aa, Homo sapiens
GRSGSAAQAEGLCK

SEQ ID NO: 85 - protein, 16 aa, Homo sapiens
STVGSSDNSSPQPLKR

SEQ ID NO: 86 - protein, 10 aa, Homo sapiens
IPSTPKLIPK

SEQ ID NO: 87 - protein, 7 aa, Homo sapiens
SLADIGK

SEQ ID NO: 88 - protein, 11 aa, Homo sapiens
SNLRSLADIGK

SEQ ID NO: 89 - protein, 14 aa, Homo sapiens
SLADIGKTVSSASR

SEQ ID NO: 90 - protein, 10 aa, Homo sapiens
AGETRFTDTR

SEQ ID NO: 91 - protein, 9 aa, Homo sapiens
SFSKEVEER

SEQ ID NO: 92 - protein, 19 aa, Homo sapiens
NKPGPNIESGNEDDDASFK

SEQ ID NO: 93 - protein, 14 aa, Homo sapiens
LGAPENSGISTLER

SEQ ID NO: 94 - protein, 10 aa, Homo sapiens
ASHSAVDITK

SEQ ID NO: 95 - protein, 8 aa, Homo sapiens
FLQPGSPR

SEQ ID NO: 96 - protein, 9 aa, Homo sapiens
SIQPQVSPR

SEQ ID NO: 97 - protein, 8 aa, Homo sapiens
LVGIISSR

SEQ ID NO: 98 - protein, 13 aa, Homo sapiens
DVDSRPEIQRLDT

SEQ ID NO: 99 - protein, 12 aa, Homo sapiens
SFTSSSPSSPSR

SEQ ID NO: 100 - protein, 11 aa, Homo sapiens
AQYEDIANRSR

SEQ ID NO: 101 - protein, 15 aa, Homo sapiens
QHYQKETESAPGSPR

SEQ ID NO: 102 - protein, 14 aa, Homo sapiens
VDHGAEIITQSPGR

SEQ ID NO: 103 - protein, 10 aa, Homo sapiens
LRAESPSPPR

SEQ ID NO: 104 - protein, 20 aa, Homo sapiens
AAPEASSPPASPLQHLLPGK

SEQ ID NO: 105 - protein, 12 aa, Homo sapiens
AITSLLGGGSPK

SEQ ID NO: 106 - protein, 13 aa, Homo sapiens
LELQGPRGSPNAR

SEQ ID NO: 107 - protein, 12 aa, Homo sapiens
GCNESFRAAIDK

SEQ ID NO: 108 - protein, 8 aa, Homo sapiens
SMGNLLEK

SEQ ID NO: 109 - protein, 9 aa, Homo sapiens
SRSMLEVPR

SEQ ID NO: 110 - protein, 11 aa, Homo sapiens
SVDISLGDSPR

SEQ ID NO: 111 - protein, 10 aa, Homo sapiens
RFVSEGDGGR

SEQ ID NO: 112 - protein, 7 aa, Homo sapiens
LMSMEMD

SEQ ID NO: 113 - protein, 11 aa, Homo sapiens
NVFTSAEELER

SEQ ID NO: 114 - protein, 13 aa, Homo sapiens
ACSMPQELPQSPR

SEQ ID NO: 115 - protein, 14 aa, Homo sapiens
TASLTSAASVDGNR

SEQ ID NO: 116 - protein, 21 aa, Homo sapiens
QPGLRQPSPSHDGSLSPLQDR

SEQ ID NO: 117 - protein, 12 aa, Homo sapiens
RDSLTGSSDLYK

SEQ ID NO: 118 - protein, 9 aa, Homo sapiens
YGMGTSVER

SEQ ID NO: 119 - protein, 15 aa, Homo sapiens
APSPAPRPVPGSPAR

SEQ ID NO: 120 - protein, 17 aa, Homo sapiens
THTTALAGRSPSPASGR

SEQ ID NO: 121 - protein, 15 aa, Homo sapiens
QQFYHSVQDLSGGSR

SEQ ID NO: 122 - protein, 16 aa, Homo sapiens
GPPSPPAPVMHSPSRK

SEQ ID NO: 123 - protein, 12 aa, Homo sapiens
VQSSPNLLAAGR

SEQ ID NO: 124 - protein, 13 aa, Homo sapiens
RVQSSPNLLAAGR

SEQ ID NO: 125 - protein, 12 aa, Homo sapiens
SFTSSSPSSPSR

SEQ ID NO: 126 - protein, 9 aa, Homo sapiens
LGSVDSFER

SEQ ID NO: 127 - protein, 14 aa, Homo sapiens
KFLEESVSMSPEER

SEQ ID NO: 128 - protein, 12 aa, Homo sapiens
RIPYAPSGEIPK

SEQ ID NO: 129 - protein, 13 aa, Homo sapiens
TQVLSPDSLFTAK

SEQ ID NO: 130 - protein, 12 aa, Homo sapiens
RLPSSPASPSPK

SEQ ID NO: 131 - protein, 8 aa, Homo sapiens
SLSPIIGK

SEQ ID NO: 132 - protein, 10 aa, Homo sapiens
QKSYTLVVAK

SEQ ID NO: 133 - protein, 10 aa, Homo sapiens
SSSMAAGLER

SEQ ID NO: 134 - protein, 19 aa, Homo sapiens
SRTSVQTEDDQLIAGQSAR

SEQ ID NO: 135 - protein, 16 aa, Homo sapiens
RTSMGGTQQQFVEGVR

SEQ ID NO: 136 - protein, 12 aa, Homo sapiens
SDFQVNLNNASR

SEQ ID NO: 137 - protein, 17 aa, Homo sapiens
SQSSHSYDDSTLPLIDR

SEQ ID NO: 138 - protein, 18 aa, Homo sapiens
SHSQASLAGPGPVDPSNR

SEQ ID NO: 139 - protein, 10 aa, Homo sapiens
SNSMLELAPK

SEQ ID NO: 140 - protein, 16 aa, Homo sapiens
DASVPLIDVTNLPTPR

SEQ ID NO: 141 - protein, 16 aa, Homo sapiens
AFGPGLQGGSAGSPAR

SEQ ID NO: 142 - protein, 9 aa, Homo sapiens
SSFTVDCSK

SEQ ID NO: 143 - protein, 9 aa, Homo sapiens
LGSFGSITR

SEQ ID NO: 144 - protein, 15 aa, Homo sapiens
QLVRGEPNVSYICSR

SEQ ID NO: 145 - protein, 10 aa, Homo sapiens
VLSTSSTLTR

SEQ ID NO: 146 - protein, 10 aa, Homo sapiens
KVIYSQPSAR

SEQ ID NO: 147 - protein, 14 aa, Homo sapiens
SRSTTELDDYSTNK

SEQ ID NO: 148 - protein, 15 aa, Homo sapiens
TSTTGVATTQSPTPR

SEQ ID NO: 149 - protein, 12 aa, Homo sapiens
RMSADMSEIEAR

SEQ ID NO: 150 - protein, 6 aa, Homo sapiens
ALSLAR

SEQ ID NO: 151 - protein, 15 aa, Homo sapiens
VIENADGSEEETDTR

SEQ ID NO: 152 - protein, 11 aa, Homo sapiens
MTESSLPSASK

SEQ ID NO: 153 - protein, 6 aa, Homo sapiens
ALSLTR

SEQ ID NO: 154 - protein, 15 aa, Homo sapiens
FRISHELDSASSEVN

SEQ ID NO: 155 - protein, 13 aa, Homo sapiens
ISHELDSASSEVN

SEQ ID NO: 156 - protein, 8 aa, Homo sapiens
ARLSYSDK

SEQ ID NO: 157 - protein, 18 aa, Homo sapiens
AKTQTPPVSPAPQPTEER

SEQ ID NO: 158 - protein, 20 aa, Homo sapiens
ASVPTIQDQASAMQLSQCAK

SEQ ID NO: 159 - protein, 9 aa, Homo sapiens
CVSCLPGQR

SEQ ID NO: 160 - protein, 19 aa, Homo sapiens
LLSDSLPPSTGTFQEAQSR

SEQ ID NO: 161 - protein, 11 aa, Homo sapiens
GLAGAVSELLR

SEQ ID NO: 162 - protein, 20 aa, Homo sapiens
VVAPTISSPVCQEQLVEAGR

SEQ ID NO: 163 - protein, 13 aa, Homo sapiens
IDSPGFKPASQQK

SEQ ID NO: 164 - protein, 12 aa, Homo sapiens
DLSGLDAETLLK

SEQ ID NO: 165 - protein, 15 aa, Homo sapiens
DPSASPGDAGEQAIR

SEQ ID NO: 166 - protein, 7 aa, Homo sapiens
FSPVTPK

SEQ ID NO: 167 - protein, 16 aa, Homo sapiens
QNPSRCSVSLSNVEAR

SEQ ID NO: 168 - protein, 14 aa, Homo sapiens
YHGHSMSDPGVSYR

SEQ ID NO: 169 - protein, 17 aa, Homo sapiens
RLSLVPDSEQGEAILPR

SEQ ID NO: 170 - protein, 20 aa, Homo sapiens
GSCNLSRVDSTTCLFPVEEK

SEQ ID NO: 171 - protein, 13 aa, Homo sapiens
VDSTTCLFPVEEK

SEQ ID NO: 172 - protein, 11 aa, Homo sapiens
GEPNVSYICSR

SEQ ID NO: 173 - protein, 21 aa, Homo sapiens
TTSFAESCKPVQQPSAFGSMK

SEQ ID NO: 174 - protein, 13 aa, Homo sapiens
HTDDEMTGYVATR

SEQ ID NO: 175 - protein, 11 aa, Homo sapiens
IRTQSFSLQER

SEQ ID NO: 176 - protein, 9 aa, Homo sapiens
ALGERVSIL

SEQ ID NO: 177 - protein, 11 aa, Homo sapiens
SASITNLSLDR

SEQ ID NO: 178 - protein, 16 aa, Homo sapiens
MCDFGISGYLVDSVAK

SEQ ID NO: 179 - protein, 13 aa, Homo sapiens
QISFKAEVNSSGK

SEQ ID NO: 180 - protein, 11 aa, Homo sapiens
YRDVSPFDHSR

SEQ ID NO: 181 - protein, 9 aa, Homo sapiens
RTSLPCIPR

SEQ ID NO: 182 - protein, 16 aa, Homo sapiens
QNPSRCSVSLSNVEAR